SEQUENCE LISTING (8 sequences)

SEQ ID NO: 1 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 2 - protein, 37 aa, Homo sapiens
HDEFERHAEGTFTSDVSSYLEGQAAKEFIAWLVKGRG

SEQ ID NO: 3 - protein, 30 aa, Homo sapiens
HAEGTFTSDVSSYLEGQAAKEFIAWLVKGR

SEQ ID NO: 4 - protein, 31 aa, Homo sapiens
HAEGTFTSDVSSYLEGQAAKEFIAWLVKGRG

SEQ ID NO: 5 - DNA, 3790 bp, Homo sapiens
  source 1..3790: mol_type = genomic DNA
ctcctcttgccacgaggtcagacggcgagttcttagagaaaaaggctgcttagctgctgcttatcatgtaacctcaaaaggaaactgatcgtctttctcatgctgtcacgtacttgggttattatcgctgattacagctggaaacaattgatttgctcttacgtatttgtgtgacttgactcttcaaacacaaaggttaacaggaagatctcgagggccctggctgaacttcaccttttggctttcttggcctgatgctgaactctcgaggttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaata

SEQ ID NO: 6 - DNA, 3923 bp, Homo sapiens
  source 1..3923: mol_type = genomic DNA
gtactggcccgcgccgtccgcccgccgacagctccctgagccagcccgggaggcagccgcgcgcagcgagccggtggcgcaggtgtcggggtcctcgagcgcccagcctgggagcatgattgtggacaagctgctggacgacagccgcggcggagaggggctgcgggacgcggcgggcggctgcggcctcatgaccagcccgctcaacctgagctacttctacggcgcgtcgccgcccgccgccgccccgggcgcctgcgacgccagctgctcggtcttgggcccctcggcgcccggctcgcccggctccgactcctccgacttctcctctgcctcgtcggtgtcctcctgcggcgccgtggagtcccggtcgagaggcggcgcccgcgccgagcgccagccagttgagccccatatgggggttggcaggcagcagagaggcccctttcaaggtgttcgggtaaagaactcagtgaaggaactcctgttgcacatccgaagtcataaacagaaggcttctggccaagctgtggatgattttaagacacaaggtgtgaacatagaacagttcagagaattgaagaacacagtatcatacagtgggaaaaggaaagggcccgattcgttgtctgatggacctgcttgcaaaaggccagctctgttgcattcccaatttttgacaccacctcaaacaccaacgcccggggagagcatggaagatgttcatctcaatgaacccaaacaggagagcagtgctgatctgcttcagaacattatcaacattaagaatgaatgcagccccgtttccctgaacacagttcaagttagctggctgaaccccgtggtggtccctcagagctcccccgcagagcagtgtcaggacttccatggagggcaggtcttttctccacctcagaaatgccaaccattccaagtcaggggctcccaacaaatgatagaccaggcttccctgtaccagtattctccacagaaccagcatgtagagcagcagccacactacacccacaaaccaactctggaatacagtccttttcccatacctccccagtcccccgcttatgaaccaaacctctttgatggtccagaatcacagttttgcccaaaccaaagcttagtttcccttcttggtgatcaaagggaatctgagaatattgctaatcccatgcagacttcctccagtgttcagcagcaaaatgatgctcacttgcacagcttcagcatgatgcccagcagcgcctgtgaggccatggtggggcacgagatggcctctgactcttcaaacacttcactgccattctcaaacatgggaaatccaatgaacaccacacagttagggaaatcactttttcagtggcaggtggagcaggaagaaagcaaattggcaaatatttcccaagaccagtttctttcaaaggatgcagatggtgacacgttccttcatattgctgttgcccaagggagaagggcactttcctatgttcttgcaagaaagatgaatgcacttcacatgctggatattaaagagcacaatggacagagtgcctttcaggtggcagtggctgccaatcagcatctcattgtgcaggatctggtgaacatcggggcacaggtgaacaccacagactgctggggaagaacacctctgcatgtgtgtgctgagaagggccactcccaggtgcttcaggcgattcagaagggagcagtgggaagtaatcagtttgtggatcttgaggcaactaactatgatggcctgactccccttcactgtgcagtcatagcccacaatgctgtggtccatgaactccagagaaatcaacagcctcattcacctgaagttcaggagcttttactgaagaataagagtctggttgataccattaagtgcctaattcaaatgggagcagcggtggaagcgaaggatcgcaaaagtggccgcacagccctgcatttggcagctgaagaagcaaatctggaactcattcgcctctttttggagctgcccagttgcctgtcttttgtgaatgcaaaggcttacaatggcaacactgccctccatgttgctgccagcttgcagtatcggttgacacaattagatgctgtccgcctgttgatgaggaagggagcagacccaagtactcggaacttggagaacgaacagccagtgcatttggttcccgatggccctgtgggagaacagatccgacgtatcctgaagggaaagtccattcagcagagagctccaccgtattagctccattagcttggagcctggctagcaacactcactgtcagttaggcagtcctgatgtatctgtacatagaccatttgccttatattggcaaatgtaagttgtttctatgaaacaaacatatttagttcactattatatagtgggttatattaaaagaaaagaagaaaaatatctaatttctcttggcagatttgcatatttcatacccaggtatctgggatctagacatctgaatttgatctcaatggtaacattgccttcaattaacagtagcttttgagtaggaaaggactttgatttgtggcacaaaacattattaatatagctattgacagtttcaaagcaggtaaattgtaaatgtttctttaagaaaaagcatgtgaaaggaaaaaggtaaatacagcattgaggcttcatttggccttagtccctgggagttactggcgttggacaggcttcagtcattggactagatgaaaggtgtccatggttagaatttgatctttgcaaactgtatataattgttatttttgtccttaaaaatattgtacatacttggttgttaacatggtcatatttgaaatgtataagtccataaaatagaaaagaacaagtgaattgttgctatttaaaaaaattttacaattcttactaaggagtttttattgtgtaatcactaagtctttgtagataaagcagatggggagttacggagttgttcctttactggctgaaagatatattcgaattgtaaagatgctttttctcatgcattgaaattatacattatttgtagggaattgcatgcttttttttttttttctcccgagacagggtcttgctctggcgcccaggctggagtacagtggcatgatcttggctcacttcagccttgacttgggctcaagtgatcctcctacctgagccttctgagtaactggaactacaggtgtgcactcctcgcctggctaattttttattttttgtacaggcaggatcttgccaccttgcccaggctggtcttgaactcctgagctcatgccatctgcctgccttagtctcccaaaatgctgggattacaggagtgagccaccatgcccggctggcagttgcatggaagagaacacctctttatggcttaccctctagaatttctaatttatgtgttctgttgaaatttttgtttttttacctttattgaaacaacaaaaagtcagtattgaaacatatcttcctgttttctgttgtcaaatgatgataatgtgccatgatgttttatatatatcattcagaaaaagttttattttttaataacattctattaacattattttgcttgccgctggcatgcctgaggaatgtatttggctttgattacacactaagtttttgtaataaatttgactcattaaaaaccttttttttttaaaaaaaaaaaaaaagaaaatctcattagtgaacttatctttgcagctgagtacttaaattctttttaaaaagataccctttggattgatcacattgtttgacccagtatgtcttgtagacacgttagttataatcaccttgtatctctaaatatggtgtgatatgaaccagtccattcacattggaaaaactgatggttttaaataaactaattcactaata

SEQ ID NO: 7 - DNA, 1678 bp, Homo sapiens
  source 1..1678: mol_type = genomic DNA
agcagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaa

SEQ ID NO: 8 - DNA, 1871 bp, Homo sapiens
  source 1..1871: mol_type = genomic DNA
gtccatctcatagcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

(19) United States
(12) Patent Application Publication
(10) Pub. No.: US 2024/0408180 A1
(43) Pub. Date: Dec. 12, 2024

(54) IONIC LIQUIDS FOR DRUG DELIVERY

(71) Applicant: PRESIDENT AND FELLOWS OF HARVARD COLLEGE, Cambridge, MA (US)

(72) Inventors: Samir MITRAGOTRI, Cambridge, MA (US); Jayoung KIM, Cambridge, MA (US); Alexander M. CURRERI, Cambridge, MA (US)

(73) Assignee: PRESIDENT AND FELLOWS OF HARVARD COLLEGE, Cambridge, MA (US)

(21) Appl. No.: 18/697,559

(22) PCT Filed: Oct. 7, 2022
(86) PCT No.: PCT/US2022/045977

§ 371 Date: Apr. 1, 2024

Related U.S. Application Data
(60) Provisional application No. 63/253,623, filed on Oct. 8, 2021.

(51) Int. Cl. A61K 38/28; A61K 9/00; A61K 39/00; A61K 47/12; A61K 47/18; C07K 16/28

(52) U.S. Cl. CPC A61K 38/28; A61K 9/0019; A61K 47/12; A61K 47/186; C07K 16/2887; A61K 2039/505; C07K 2317/94

(57) ABSTRACT

The technology described herein is directed to ionic liquids and methods of drug delivery.

### CROSS-REFERENCE TO RELATED APPLICATIONS

This application claims benefit under 35 U.S.C. § 119(e) of U.S. Provisional Application No. 63/253,623 filed Oct. 8, 2021, the contents of which are incorporated herein by reference in their entirety.

### TECHNICAL FIELD

The technology described herein relates to ionic liquids for stabilization and delivery of active compounds.

### BACKGROUND

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. These toxic and irritant effects are severe enough to mitigate any increase in the uptake or performance of the active compound.

### SUMMARY

Ionic liquids are a potential solution to drug delivery obstacles. Described herein are novel ionic liquids with surprisingly improved drug delivery kinetics, e.g., as compared to “first-generation” ionic liquids such as choline:geranic acid (CAGE). As demonstrated herein, the inventors have identified characteristics of ionic liquids that provide surprising superior active compound uptake kinetics for certain types of active compounds. Accordingly, compositions and methods relating to these ionic liquids (ILs) with unexpectedly high efficacy are described herein.

In one aspect of any of the embodiments, described herein is a composition comprising at least one ionic liquid comprising:
an anion which is at least one of:
   a) a carboxylic acid which is not a fatty acid; and
   b) a hydrophobic anion comprising carboxylic acid having a pKa of at least 4.0 and a Log P of at least 1.0;
a cation which is a quaternary ammonium comprising an ester group, e.g., acetylcholine.

In some embodiments of any of the aspects, the anion is a carboxylic acid which is not a fatty acid. In some embodiments of any of the aspects, the anion has a Log P of less than 1.0. In some embodiments of any of the aspects, the fatty acid comprises an aliphatic chain of no more than 3 carbons. In some embodiments of any of the aspects, the anion comprises only one carboxylic acid group (e.g., R—COOH group). In some embodiments of any of the aspects, the anion is selected from the group consisting of: lactic acid; glycolic acid; malonic acid; maleic acid; glutaric acid; citric acid; gluconic acid; and adipic acid. In some embodiments of any of the aspects, the anion is selected from the group consisting of: propanoic acid; lactic acid; glycolic acid; malonic acid; maleic acid; glutaric acid; citric acid; gluconic acid; and adipic acid. In some embodiments of any of the aspects, the anion is maleic acid. In some embodiments of any of the aspects, the anion is propanoic acid.

In some embodiments of any of the aspects, the anion is a hydrophobic anion comprising carboxylic acid having a pKa of at least 4.0 and a Log P of at least 1.0 and has a pKa of at least 4.5. In some embodiments of any of the aspects, the anion has a pKa of at least 5.0. In some embodiments of any of the aspects, the anion comprises a carbon chain of at least 8 carbons. In some embodiments of any of the aspects, the anion comprises a carbon chain with an 8 carbon backbone. In some embodiments of any of the aspects, the anion is geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, or hexenoic acid. In some embodiments of any of the aspects, the anion is hexenoic acid.

In some embodiments of any of the aspects, the ionic liquid comprises a ratio of cation to anion of from about 2:1 to about 1:1. In some embodiments of any of the aspects, the ionic liquid comprises a ratio of cation to anion of about 2:1. In some embodiments of any of the aspects, the ionic liquid has a cation:anion ratio of less than 1:1. In some embodiments of any of the aspects, the ionic liquid has a cation:anion ratio with an excess of cation. In some embodiments of any of the aspects, the composition comprises a first ionic liquid and at least a second ionic liquid. In some embodiments of any of the aspects, the composition comprises a first ionic liquid comprising an anion which is at least one of: a) a carboxylic acid which is not a fatty acid; and b) a hydrophobic anion comprising carboxylic acid having a pKa of at least 4.0 and a Log P of at least 1.0; and a cation which is a quaternary ammonium comprising an ester group (e.g, acetylcholine) and at least a second ionic liquid. In some embodiments of any of the aspects, the composition comprises a first ionic liquid and at least a second ionic liquid, each comprising an anion which is at least one of: a) a carboxylic acid which is not a fatty acid; and b) a hydrophobic anion comprising carboxylic acid having a pKa of at least 4.0 and a Log P of at least 1.0; and a cation which is a quaternary ammonium comprising an ester group (e.g., acetylcholine). In some embodiments of any of the aspects, the first ionic liquid and the second ionic liquid each comprise a different anion.

In some embodiments of any of the aspects, the composition further comprises at least one active compound in combination with the at least one ionic liquid. In some embodiments of any of the aspects, the active compound comprises a polypeptide. In some embodiments of any of the aspects, the polypeptide is an antibody or antibody reagent. In some embodiments of any of the aspects, the active compound has a molecular weight of greater than 450. In some embodiments of any of the aspects, the active compound has a molecular weight of greater than 500. In some embodiments of any of the aspects, the active compound comprises insulin, acarbose, ruxolitinib, or a GLP-1 polypeptide or mimetic or analog thereof. In some embodiments of any of the aspects, the anion is a hydrophobic anion comprising carboxylic acid having a pKa of at least 4.0 and a Log P of at least 1.0, and the active compound comprises an antibody or antibody reagent. In some embodiments of any of the aspects, the active compound comprises a nucleic acid. In some embodiments of any of the aspects, the nucleic acid is an inhibitory nucleic acid. In some embodiments of any of the aspects, the nucleic acid is a siRNA, pDNA, or mRNA. In some embodiments of any of the aspects, the anion is a hydrophobic anion comprising carboxylic acid having a pKa of at least 4.0 and a Log P of at least 1.0 and the active agent is a nucleic acid.

In some embodiments of any of the aspects, the ionic liquid is at a concentration of at least 0.10% w/v. In some embodiments of any of the aspects, the ionic liquid is at a concentration of from about 10 to about 70% w/v. In some embodiments of any of the aspects, the ionic liquid is at a concentration of from about 30 to about 50% w/v. In some embodiments of any of the aspects, the ionic liquid is at a concentration of from about 30 to about 40% w/v. In some embodiments of any of the aspects, the ionic liquid is at a concentration of less than 10% w/v. In some embodiments of any of the aspects, the composition is formulated for administration transdermally, to a mucus membrane, orally, subcutaneously, intradermally, parenterally, intratumorally, or intravenously. In some embodiments of any of the aspects, the composition is formulated for transdermal administration. In some embodiments of any of the aspects, the mucus membrane is nasal, oral, or vaginal. In some embodiments of any of the aspects, the active compound is provided at a dosage of 1-40 mg/kg. In some embodiments of any of the aspects, the composition further comprises at least one non-ionic surfactant. In some embodiments of any of the aspects, the composition further comprises a pharmaceutically acceptable carrier. In some embodiments of any of the aspects, the composition is provided in a degradable capsule. In some embodiments of any of the aspects, the composition is an admixture. In some embodiments of any of the aspects, the composition is provided in one or more nanoparticles. In some embodiments of any of the aspects, the composition comprises one or more nanoparticles comprising the active compound, the nanoparticles in solution or suspension in a composition comprising the ionic liquid.

In one aspect of any of the embodiments, described herein is a method of administering at least one active compound to a subject, the method comprising administering a composition described herein. In one aspect of any of the embodiments, described herein is a composition as described herein for use in a method of administering at least one active compound to a subject. In some embodiments of any of the aspects, the composition is administered once. In some embodiments of any of the aspects, the composition is administered in multiple doses. In some embodiments of any of the aspects, the administering is transdermally, to a mucus membrane, orally, subcutaneously, intradermally, parenterally, intratumorally, or intravenously

### BRIEF DESCRIPTION OF THE DRAWINGS

FIGS. 1A-1B depict scattering data demonstrating insulin stability in ILs.

FIG. 2 depicts circular dichroism demonstrating insulin secondary structure in ILs.

FIG. 3 depicts in vivo delivery of insulin in ILs.

FIGS. 4A-4B depict in vivo delivery of antibodies in ILs.

FIG. 5 depicts antibody stability in ILs.

FIG. 6 depicts in vitro transfection of mRNA.

FIGS. 7A-7D depict the determination of SPADE formulation. (FIG. 7A) Average decrease in transmittance of insulin-DES formulations stored at 37° C. with continuous shaking for 50 hours. The shaded gray represents the region in which formulations were considered unstable. (n=3) (FIG. 7B) Average decrease in transmittance of insulin-DES formulations that were stable in (FIG. 7A) stored at 4° C. for 28 days. The shaded gray represents the region in which formulations were considered unstable. (n=3) (FIG. 7C) Circular dichroism for insulin-DES formulations that were stable in (B) compared to the control (n=5). (FIG. 7D) Insulin transport across HUVEC monolayer on transwell cell culture inserts (n=3). Statistical significance was determined with a t-test. *p<0.05, *p<0.01.,

FIGS. 8A-8C depict the SPADE mechanism of action. (FIG. 8A) A schematic representation of SPADE-insulin (right) as compared to the non-DES-containing control (left) when administer in the subcutaneous space. (FIG. 8B) Fluorescence polarization for the control and SPADE-insulin when mixed with collagen (Control n=6, SPADE-insulin n=5). (FIG. 8C) Average diameter as measured with DLS versus time of Humalog and SPADE-insulin when mixed with collagen (n−3). Statistical significance was determined with a t-test. *p<0.05.

FIGS. 9A-9E depict SPADE-insulin pharmacokinetics. (FIG. 9A) PK study design for SPADE-insulin versus Humalog including subcutaneous injections of 1 U/kg insulin (red arrow) and blood sampling schedule (purple arrow). (FIG. 9B) Insulin serum concentration against time for the first 60 minutes of the PK study. (FIG. 9C) AUC 5 minutes after injection. (FIG. 9D) AUC 10 minutes after injection. (FIG. 9E) Percent injected does absorbed of each formulation after 240 minutes. (Humalog n=6, SPADE-insulin n=5) Statistical significance was determined with a t-test. *p<0.05.

FIGS. 10A-10M depict SPADE safety assessment. (FIG. 10A) Safety study designs for SPADE versus saline control including injection site toxicity (left) and repeat dosing study (right). The studies incorporated subcutaneous injection (red arrow), injection site tissue collection (yellow arrow), blood and vital organ collection (green arrow), and euthanization (black arrow). (FIGS. 10B-10E) Injection site H&E images for indicated formulation and time points. Scale bars, 200 μm. (FIG. 10F) aspartate aminotransferase serum concentrations (FIG. 10G) alanrine aminotransferase serum concentrations groups (FIG. 10H) blood urea nitrogen serum concentrations (FIG. 10I) creatinine serum concentrations (FIG. 10J) white blood cell counts (FIG. 10K) red blood cell counts (FIG. 10L) platelet counts (FIG. 10M) lymphocyte counts for control (n=4) and SPADE (n=5) treated groups. Dotted lines represent the expected range for metric of interest.³⁸ Statistical significance was determined with a t-test. *p<0.05.

FIGS. 11A-11E depict SPADE-mAb stability, pharmacokinetics, and bioavailability. (FIG. 11A) SDS-PAGE gel electrophoresis for the assessment of SPADE-mAb stability for indicated DES concentrations and 37° C. incubation times. The protein ladder is labeled with corresponding molecular weights (kDa). Red arrows indicate antibody aggregates. (FIG. 11B) Circular dichroism spectra for 24 hour incubated formulations from (A) versus a control stable antibody formulation. (FIG. 11C) PK study design for SPADE-mAb versus control including subcutaneous injections of 10 mg/kg rituximab (red arrow) and blood sampling schedule (purple arrow). (FIG. 11D) Rituximab serum concentration versus time and (FIG. 11E) AUC versus time for the 49 day antibody PK study for control (n=6) and SPADE-mAb (n=5 until day 21, n=4 day 28 to 49). Statistical significance was determined with a t-test. *p<0.05.

FIGS. 12A-12B depict a summary of FDA approved new molecular entities (NMEs), biological license applications (BLAs), and antibodies since 2007. (FIG. 12A) The trend in NME and BLA (including antibodies which are noted in purple) approvals since 2007. (FIG. 12B) The cumulative BLA and antibody (a subclass of BLAs) approved since 2007. This data was adopted from multiple reports by B. Hughes and A. Mullard.¹⁻¹⁵

FIG. 13 depicts the transmittance (%) for insulin-DES formulations and relevant controls. The dotted line marks that 80% transmittance that was used as the minimum threshold to proceed to next study.

FIG. 14 depicts high tension voltage (V) vs. wavelength that was measured for samples during CD experiments. The 500 V maximum threshold (marked by the dotted line) is used as a check to confirm CD spectra integrity.

FIG. 15 depicts cell viability (%) vs logarithm of DES concentration (mM). The dotted line represents the maximum viable concentration (0.15%).

FIG. 16 depicts the area under the curve (AUC) values for the insulin pharmacokinetic study at timepoints between 15 and 240 minutes.

FIGS. 17-18 depict additional whole blood analysis results, including various leukocyte levels, hematocrit (HCT), mean corpuscular volume (MCV), mean corpuscular hemoglobin (MCH), mean corpuscular hemoglobin concentration (MCHC), and hemoglobin (HGB). Statistical significance was determined with t-test. *p<0.05, **p<0.01 ***p<0.001, *<00001

FIGS. 19-21 depict additional serum analysis results, including a variety of serum proteins, enzymes, alkaline phosphatase (ALP), ions, and other biomarkers. Only creatine kinase levels were significantly higher in the SPADE group, however this is likely due the release associated with cardiac puncture that was performed on some mice in the group. Statistical significance was determined with t-test. *p<0.05, **p<0.01, ***p<0001, ****p<0,0001.

FIG. 22 depicts H&E staining of vital organs from the toxicity study in which mice were dosed multiple times with SPADE. Scale bars, 200 μm.

FIG. 23 depicts fold-change of AUC (AUC-SPADE-mAb/AUC-Control) for the first 14 days of the rituximab study.

### DETAILED DESCRIPTION

The data provided herein demonstrate that certain anions provide superior drug delivery characteristics when combined with an a quaternary ammonium comprising an ester group (e.g., acetylcholine) cation, e.g., as compared to a choline cation. In particular, subcutaneous administration of drugs is hampered by the fact that many drugs will interact with the extracellular matrix. For example, insulin will interact with collagen and other elements of the extracellular matrix after subcutaneous administration, reducing the amount and speed of drug delivery to the bloodstream. This reduces the dose and kinetics of subcutaneous administration. The inventors have discovered herein that certain ionic liquids function as matrix-interaction reducing agents. That is, when a drug is administered subcutaneously in combination with certain ionic liquids described herein, the interaction of the drug with the extracellular matrix is reduced, and the drug more quickly and efficiently enters the bloodstream. Accordingly, in one aspect of any of the embodiments, described herein is a composition comprising at least one ionic liquid comprising 1) an anion which is at least one of:
a) a carboxylic acid which is not a fatty acid; and
b) a hydrophobic anion comprising carboxylic acid having a pKa of at least 4.0 and a Log P of at least 1.0; and
   2) a cation which is a quaternary ammonium comprising an ester group (e.g., acetylcholine).

The term “ionic liquids (ILs)” as used herein refers to organic salts or mixtures of organic salts which are in liquid state at room temperature. This class of solvents has been shown to be useful in a variety of fields, including in industrial processing, catalysis, pharmaceuticals, and electrochemistry. The ionic liquids contain at least one anionic and at least one cationic component. Ionic liquids can comprise an additional hydrogen bond donor (i.e. any molecule that can provide an —OH or an —NH group), examples include but are not limited to alcohols, fatty acids, and amines. The at least one anionic and at least one cationic component may be present in any molar ratio. Exemplary molar ratios (cation:anion) include but are not limited to 1:1, 1:2, 2:1, 1:3, 3:1, 2:3, 3:2, and ranges between these ratios. For further discussion of ionic liquids, see, e.g., Hough, et al, “The third evolution of ionic liquids: active pharmaceutical ingredients”, New Journal of Chemistry, 31:1429 (2007) and Xu, et al., “Ionic Liquids: Ion Mobilities, Glass Temperatures, and Fragilities”, Journal of Physical Chemistry B, 107(25): 6170-6178 (2003); each of which is incorporated by reference herein in its entirety. In some embodiments of any of the aspects, the ionic liquid or solvent exists as a liquid below 100° C. In some embodiments of any of the aspects, the ionic liquid or solvent exists as a liquid at room temperature.

As demonstrated herein, ionic liquids combining a a quaternary ammonium comprising an ester group (e.g., acetylcholine) cation and anions with certain physical characteristics provide superior or improved drug delivery characteristics, e.g., as compared to ionic liquids comprising the same anion and a choline cation. In some embodiments, improved drug delivery characteristics comprise reduced denaturation or degradation of the cargo molecule. In some embodiments, improved drug delivery characteristics comprise increased ability to cross biological barriers (e.g., increased permeability). In some embodiments of any of the aspects, the improved drug delivery characteristics are for insulin. In some embodiments of any of the aspects, the improved drug delivery characteristics are for large polypeptide (e.g., antibody) cargo molecules. In some embodiments of any of the aspects, the improved drug delivery characteristics are for nucleic acid cargo molecules.

In some embodiments of any of the aspects, the anion of an IL described herein is hydrophobic.

In some embodiments of any of the aspects, the anion of an IL described herein comprises, consists of, or consists essentially of a carboxylic acid. In some embodiments of any of the aspects, the anion of an IL described herein comprises, consists of, or consists essentially of a carboxylic acid which is not a fatty acid.

A carboxylic acid is a compound having the structure of Formula I, wherein R can be any group.

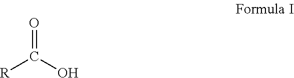

Generally, the anion is R—X⁻, where X is CO₂⁻, SO₃⁻, OSO₃²⁻ or OPO₃²⁻; and R is optionally substituted C₁-C₁₀alkyl, optionally substituted C₂-C₁₀alkenyl, or optionally substituted C₂-C₁₀ alkynyl, optionally substituted aryl, or optionally substituted heteroaryl.

In some embodiments, R is an optionally substituted linear or branched C₁-C₉alkyl. For example, R is a C₁-C₉alkyl optionally substituted with 1, 2, 3, 4, 5 or 6 substituents independently selected from the group consisting of C₁-C₃alkyl, hydroxy (OH), halogen, oxo (═O), carboxy (CO₂), cyano (CN) and aryl. In some embodiments, R is a C₁-C₆alkyl optionally substituted with 1, 2, 3, 4 or 5 substituents independently selected from the group consisting of C₁-C₃alkyl, hydroxy, carboxy and phenyl. Preferably, R is a C₁-C₅ alkyl, optionally substituted with 1, 2, 3, 4 or 5 substituents independently selected from the group consisting of methyl, ethyl, hydroxyl, carboxy, and phenyl. Exemplary alkyls for R include, but are not limited to, methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl and nonyl.

In some embodiments, R is an optionally substituted linear or branched C₂-C₈alkenyl. For example, R is a C₂-C₉alkenyl optionally substituted with 1, 2, 3, 4, 5 or 6 substituents independently selected from the group consisting of C₁-C₃alkyl, hydroxy, halogen, oxo, carboxy, cyano and aryl. In some embodiments, R is a C₂-C₆alkenyl optionally substituted with 1, 2, 3, 4 or 5 substituents independently selected from the group consisting of C₁-C₃alkyl, hydroxy, carboxy and phenyl. Preferably, R is a C₁-C₅alkenyl, optionally substituted with 1, 2, 3, 4 or 5 substituents independently selected from the group consisting of methyl, ethyl, hydroxyl, carboxy, and phenyl. Exemplary alkenyls for R include, but are not limited to, ethenyl, 2-carboxyethenyl, 1-methylpropenyl and 2-methylpropenyl.

In some embodiments, R is an optionally substituted aryl or heteroaryl. For example, R is an aryl or heteroayl optionally substituted with 1, 2, 3, 4, 5 or 6 substituents independently selected from the group consisting of C₁-C₃alkyl, hydroxy, halogen, oxo, carboxy, cyano and aryl. In some embodiments, R is an aryl optionally substituted with 1, 2, 3, 4 or 5 substituents independently selected from the group consisting of C₁-C₃alkyl, hydroxy, carboxy and phenyl. Preferably R is a phenyl substituted with 1, 2 or 3 substituents independently selected from the group consisting of methyl, ethyl, hydroxyl, carboxy, and phenyl. Exemplary aryls for R include, but are not limited to, phenyl, 2-hydroxyphenyl, 3-hydroxyphenyl, 4-hydroxyphenyl, dihydroxyphenyl, trihydroxyphenyl, 3,4,5-trihydroxyphenyl, and 1,1-biphen-4-yl.

In some embodiments, X is CO₂ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In some other embodiments, X is OSO₃⁻ and R is methyl, carboxymethyl, hydroxymethyl, ethyl, 1-hydroxyethyl, 2-phenylethyl, propyl, prop-2-yl, 1-methylpropyl, 2-methylpropyl, 3-carboxypropyl, 2,3-dicarboxymethyl-2-hydroxypropyl, butyl, pentyl, 1,2,3,4,5-pentahydroxypentyl, hexyl, 2-ethylhexyl, nonyl, ethenyl, 2-carboxyethenyl, 1-methylpropenyl, 2-methylpropenyl, 3,4,5-trihydroxyphenyl, or 1,1-biphen-4-yl. In yet some other embodiments, X is OPO₃²⁻ or SO₃⁻ and R is 2-hydroxyphenyl, 3-hydroxyphenyl or 4-hydroxyphenyl.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals, having the number of carbon atoms designated (i.e., C₁-C₁₀ means one to ten carbons). An alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An “alkenyl” is an unsaturated alkyl group is one having one or more double bonds bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), and the higher homologs and isomers.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Exemplary aryl and heteroaryl groups include, but are not limited to, phenyl, 4-nitrophenyl, 1-naphthyl, 2-naphthyl, biphenyl, 4-biphenyl, pyrrole, 1-pyrrolyl, 2-pyrrolyl, 3-pyrrolyl, pyrazole, 3-pyrazolyl, imidazole, imidazolyl, 2-imidazolyl, 4-imidazolyl, benzimidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, thiazolyl, 2-thiazolyl, 4-thiazolyl, 5-thiazolyl, 2-furyl, 3-furyl, 2-thienyl, 3-thienyl, pyridine, 2-pyridyl, naphthyridinyl, 3-pyridyl, 4-pyridyl, benzophenonepyridyl, pyridazinyl, pyrazinyl, 2-pyrimidyl, 4-pyrimidyl, pyrimidinyl, 5-benzothiazolyl, purinyl, 2-benzimidazolyl, indolyl, 5-indolyl, quinoline, quinolinyl, 1-isoquinolyl, 5-isoquinolyl, 2-quinoxalinyl, 5-quinoxalinyl, 3-quinolyl, 6-quinolyl, furan, furyl or furanyl, thiophene, thiophenyl or thienyl, diphenylether, diphenylamine, and the like.

The term “optionally substituted” means that the specified group or moiety is unsubstituted or is substituted with one or more (typically 1, 2, 3, 4, 5 or 6 substituents) independently selected from the group of substituents listed below in the definition for “substituents” or otherwise specified. The term “substituents” refers to a group “substituted” on a substituted group at any atom of the substituted group. Suitable substituents include, without limitation, halogen, hydroxy, caboxy, oxo, nitro, haloalkyl, alkyl, alkenyl, alkynyl, alkaryl, aryl, heteroaryl, cyclyl, heterocyclyl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

As used herein, “fatty acid” refers to a carboxylic acid wherein R comprises a saturated or unsaturated aliphatic chain, e.g., R has the formula CₙH₂ₙ₊₁. In some embodiments of any of the aspects, the fatty acid is a monocarboxylic acid. The fatty acid can be natural or synthetic. The aliphatic chain of the fatty acid can be saturated, unsaturated, branched, straight, and/or cyclic. In some embodiments of any of the aspects, the aliphatic chain does not comprise an aromatic group. In some embodiments of any of the aspects, the aliphatic chain comprises, consists of, or consists essentially of an alkyl or alkene chain.

Exemplary carboxylic acids which are not fatty acids can include, but are not limited to propanoic acid; lactic acid; glycolic acid; malonic acid; maleic acid; glutaric acid; citric acid; gluconic acid; and adipic acid.

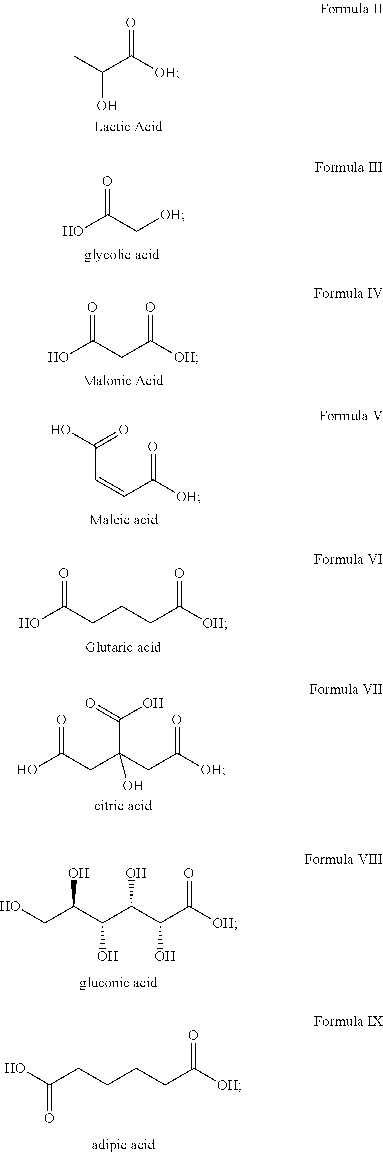

In some embodiments of any of the aspects, the carboxylic acid which is not a fatty acid is lactic acid; glycolic acid; malonic acid; maleic acid; glutaric acid; citric acid; gluconic acid; or adipic acid. In some embodiments of any of the aspects, the carboxylic acid which is not a fatty acid is maleic acid. In some embodiments of any of the aspects, the carboxylic acid which is not a fatty acid is propanoic acid; lactic acid; glycolic acid; malonic acid; maleic acid; glutaric acid; citric acid; gluconic acid; or adipic acid. In some embodiments of any of the aspects, the carboxylic acid which is not a fatty acid is propanoic acid.

In some embodiments, the carboxylic acid which is not a fatty acid comprises no more than 5 carbons in the R group, either in a straight or branched configuration. In some embodiments, the carboxylic acid which is not a fatty acid comprises no more than 3 carbons in the R group, either in a straight or branched configuration. In some embodiments, the carboxylic acid which is not a fatty acid comprises a hydroxy group in the R group. In some embodiments, the carboxylic acid which is not a fatty acid comprises one or more carboxylic acids in the R group.

In some embodiments, the carboxylic acid which is not a fatty acid comprises no more than 5 carbons in the R group, either in a straight or branched configuration, and comprises a hydroxy group in the R group. In some embodiments, the carboxylic acid which is not a fatty acid comprises no more than 3 carbons in the R group, either in a straight or branched configuration, and comprises a hydroxy group in the R group. In some embodiments, the carboxylic acid which is not a fatty acid comprises 1-5 carbons in the R group, either in a straight or branched configuration, and comprises a hydroxy group in the R group.

In some embodiments, the carboxylic acid which is not a fatty acid comprises no more than 5 carbons in the R group, either in a straight or branched configuration, and comprises one or more carboxylic acid groups in the R group. In some embodiments, the carboxylic acid which is not a fatty acid comprises no more than 3 carbons in the R group, either in a straight or branched configuration, and comprises one or more carboxylic acid groups in the R group. In some embodiments, the carboxylic acid which is not a fatty acid comprises 1-5 carbons in the R group, either in a straight or branched configuration, and comprises one or more carboxylic acid groups in the R group.

In some embodiments, the carboxylic acid which is not a fatty acid comprises 1-5 carbons in the R group, either in a straight or branched configuration, and comprises one carboxylic acid group in the R group. In some embodiments, the carboxylic acid which is not a fatty acid comprises 1-3 carbons in the R group, either in a straight or branched configuration, and comprises one carboxylic acid group in the R group.

When the number of carbons in a chain is referred to herein, it is contemplated that the entire number of carbons in the chain (including branches) is referred to. In the case of a straight chain, this is the same as the carbon chain length. In the case of a branched chain, “chain length” refers to the longest carbon chain branch of the branched chain.

In some embodiments, the anion comprises one carboxylic acid group.

Hydrophobicity may be assessed by analysis of log P. “Log P” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.

$$Partition Coefficient , P = [ Organic ] / [ Aqueous ] \\ where [ ] = concentration$$

$$Log P = {log}_{10} ( Partition Coefficient ) = {log}_{10} P$$

In practice, the Log P value will vary according to the conditions under which it is measured and the choice of partitioning solvent. A Log P value of 1 means that the concentration of the compound is ten times greater in the organic phase than in the aqueous phase. The increase in a log P value of 1 indicates a ten fold increase in the concentration of the compound in the organic phase as compared to the aqueous phase.

In some embodiments of any of the aspects, the anion which is a carboxylic acid which is not a fatty acid has a Log P of less than 1.0. In some embodiments of any of the aspects, the anion which is a carboxylic acid which is not a fatty acid has a Log P of less than 0.80. In some embodiments of any of the aspects, the anion which is a carboxylic acid which is not a fatty acid has a Log P of less than 0.75. In some embodiments of any of the aspects, the anion which is a carboxylic acid which is not a fatty acid has a Log P of less than 0.50. In some embodiments of any of the aspects, the anion which is a carboxylic acid which is not a fatty acid has a Log P of less than 0.25. In some embodiments of any of the aspects, the anion which is a carboxylic acid which is not a fatty acid has a Log P of less than 0.

In some embodiments of any of the aspects, the anion of an IL described herein has a pKa of less than 4.0. In some embodiments of any of the aspects, the anion of an IL described herein has a pKa of less than 4.0 and a Log P of less than 1.0. Exemplary anions are provided in Table 1 below.

TABLE 1

| | LogP | pKa |
|---|---|---|
| Glycolic acid | −1.11 | 3.8 |
| Propanoic acid (propionic acid) | 0.33 | 4.88 |
| Isoburtyric acid | 0.94 | 4.84 |
| Butyric acid | 0.79 | 4.82 |
| Gallic acid | 0.70 | 4.40 |
| Lactic acid | −0.72 | 3.86 |
| Malonic acid | −0.81 | 2.8 |
| Decanoic Acid | 4.09 | 4.9 |
| Maleic acid | −0.48 | 1.83 |
| Glutaric acid | −0.29 | 4.34 |
| Citric acid | −1.64 | 2.79 |
| 3,3-dimethylacrylic acid | 1.2 | 5.02 |
| Gluconic acid | −3.4 | 3.39 |
| Adipic acid | 0.08 | 4.4 |
| 2-Ethylhexyl sulfate | 3.10 | |
| 4-hydroxybenzenesulfonic acid | 0.2 | 9.11 |
| Isovaleric acid | 1.16 | 4.77 |
| Hydrocinnamic acid | 1.84 | 4.66 |
| Phenylphosphoric acid | 1.05 | 9.99 |
| Biphenyl-3-carboxylic acid | 3.5 | 4.14 |

In some embodiments of any of the aspects, the anion is an alkane. In some embodiments of any of the aspects, the anion is an alkene. In some embodiments of any of the aspects, the anion comprises a single carboxyl group. In some embodiments of any of the aspects, the carbon chain of the carboxylic acid comprises one or more substituent groups. In some embodiments of any of the aspects, the carbon chain backbone of the carboxylic acid comprises one or more substituent groups, wherein each substituent group comprises at least one carbon atom. In some embodiments of any of the aspects, the carbon chain backbone of the carboxylic acid comprises one or more substituent groups, wherein at least one substituent group comprises a methyl group. In some embodiments of any of the aspects, the carbon chain backbone of the carboxylic acid comprises two substituent groups, wherein each substituent group comprises at least one carbon atom. In some embodiments of any of the aspects, the carbon chain backbone of the carboxylic acid comprises two substituent groups, wherein one substituent group comprises a methyl group. In some embodiments of any of the aspects, the carbon chain backbone of the carboxylic acid comprises two substituent groups, wherein each substituent group comprises a methyl group.

In some embodiments of any of the aspects, the anion is an unsubstituted alkane. In some embodiments of any of the aspects, the anion is an unsubstituted alkene. In some embodiments of any of the aspects, the carbon chain backbone of the carboxylic acid comprises one or more substituent groups. In some embodiments of any of the aspects, the carbon chain of the carboxylic acid comprises one or more substituent groups, wherein each substituent group comprises at least one carbon atom. In some embodiments of any of the aspects, the carbon chain of the carboxylic acid comprises one or more substituent groups, wherein each substituent group is alkyl, aryl, heteroalkayl, heteroaryl, alkane, or alkene. In some embodiments of any of the aspects, the carbon chain of the carboxylic acid comprises one or more substituent groups, wherein each substituent group is unsubstituted alkyl, unsubstituted aryl, unsubstituted heteroalkayl, unsubstituted heteroaryl, unsubstituted alkane, or unsubstituted alkene.

In one aspect of any of the embodiments, described herein is a composition comprising at least one ionic liquid comprising, consisting of, or consisting essentially of 1) an anion which is a carboxylic acid which is not a fatty acid, and 2) a cation which is a quaternary ammonium comprising an ester group (e.g., acetylcholine). In one aspect of any of the embodiments, described herein is a composition comprising at least one ionic liquid comprising, consisting of, or consisting essentially of 1) an anion with a Log P of less than 1.0 and which is a carboxylic acid which is not a fatty acid, and 2) a cation which is a quaternary ammonium comprising an ester group (e.g., acetylcholine).

In one aspect of any of the embodiments, described herein is a composition comprising at least one ionic liquid comprising, consisting of, or consisting essentially of 1) a hydrophobic anion comprising carboxylic acid having a pKa of at least 4.0 and a Log P of at least 1.0, and 2) a cation which is a quaternary ammonium comprising an ester group (e.g., acetylcholine). In one aspect of any of the embodiments, described herein is a composition comprising at least one ionic liquid comprising, consisting of, or consisting essentially of 1) a hydrophobic anion comprising carboxylic acid having a pKa of at least 4.0 and a Log P of at least 1.0, and 2) a cation which is a quaternary ammonium comprising an ester group (e.g., acetylcholine).

In some embodiments of any of the aspects, the anion of an IL described herein is hydrophobic. In some embodiments of any of the aspects, the anion of an IL described herein is comprises a carboxylic acid.

In some embodiments of any of the aspects, the anion of an IL described herein is has a pKa of at least 4.0, e.g., 4.0 or greater. In some embodiments of any of the aspects, the anion of an IL described herein is has a pKa of at least 4.5, e.g., 4.5 or greater. In some embodiments of any of the aspects, the anion of an IL described herein is has a pKa of at least 5.0, e.g., 5.0 or greater. In some embodiments of any of the aspects, the anion of an IL described herein is has a pKa of at about least 4.0, e.g., about 4.0 or greater. In some embodiments of any of the aspects, the anion of an IL described herein is has a pKa of at least about 4.5, e.g., about 4.5 or greater. In some embodiments of any of the aspects, the anion of an IL described herein is has a pKa of at least about 5.0, e.g., about 5.0 or greater.

In some embodiments of any of the aspects, the anion has a pKa of at least 4.895. In some embodiments of any of the aspects, the anion has a pKa of 4.5-5.5. In some embodiments of any of the aspects, the anion has a pKa of 4.895-5.19.

In some embodiments of any of the aspects, the anion has a pKa of at least about 4.895. In some embodiments of any of the aspects, the anion has a pKa of about 4.5 to about 5.5. In some embodiments of any of the aspects, the anion has a pKa of about 4.895 to about 5.19.

In some embodiments of any of the aspects, the anion of an IL described herein is has a Log P of at least 1.0, e.g., 1.0 or greater. In some embodiments of any of the aspects, the anion of an IL described herein is has a Log P of at least 2.0, e.g., 2.0 or greater. In some embodiments of any of the aspects, the anion of an IL described herein is has a Log P of at least 2.5 e.g., 2.5 or greater. In some embodiments of any of the aspects, the anion of an IL described herein is has a Log P of at least 2.75, e.g., 2.75 or greater. In some embodiments of any of the aspects, the anion of an IL described herein is has a Log P of at least about 1.0, e.g., about 1.0 or greater. In some embodiments of any of the aspects, the anion of an IL described herein is has a Log P of at least about 2.0, e.g., about 2.0 or greater. In some embodiments of any of the aspects, the anion of an IL described herein is has a Log P of at least about 2.5 e.g., about 2.5 or greater. In some embodiments of any of the aspects, the anion of an IL described herein is has a Log P of at least about 2.75, e.g., about 2.75 or greater.

In some embodiments of any of the aspects, the anion of an IL described herein is has a pKa of at least 4.0 and a Log P of at least 1.0. In some embodiments of any of the aspects, the anion of an IL described herein is has a pKa of at least 4.0 and a Log P of at least 2.0. In some embodiments of any of the aspects, the anion of an IL described herein is has a pKa of at least 4.0 and a Log P of at least 2.5. In some embodiments of any of the aspects, the anion of an IL described herein is has a pKa of at least 4.0 and a Log P of at least 2.75.

In some embodiments of any of the aspects, the anion of an IL described herein is has a pKa of at least 4.5 and a Log P of at least 1.0. In some embodiments of any of the aspects, the anion of an IL described herein is has a pKa of at least 4.5 and a Log P of at least 2.0. In some embodiments of any of the aspects, the anion of an IL described herein is has a pKa of at least 4.5 and a Log P of at least 2.5. In some embodiments of any of the aspects, the anion of an IL described herein is has a pKa of at least 4.5 and a Log P of at least 2.75.

In some embodiments of any of the aspects, the anion of an IL described herein is has a pKa of at least 5.0 and a Log P of at least 1.0. In some embodiments of any of the aspects, the anion of an IL described herein is has a pKa of at least 5.0 and a Log P of at least 2.0. In some embodiments of any of the aspects, the anion of an IL described herein is has a pKa of at least 5.0 and a Log P of at least 2.5. In some embodiments of any of the aspects, the anion of an IL described herein is has a pKa of at least 5.0 and a Log P of at least 2.75.

In some embodiments of any of the aspects, the anion has a Log P of at least 2.75. In some embodiments of any of the aspects, the anion has a Log P of at least 2.8. In some embodiments of any of the aspects, the anion has a Log P of 2.5-3.5. In some embodiments of any of the aspects, the anion has a Log P of 2.8-3.01.

In some embodiments of any of the aspects, the anion has a Log P of at least about 2.75. In some embodiments of any of the aspects, the anion has a Log P of at least about 2.8. In some embodiments of any of the aspects, the anion has a Log P of about 2.5 to about 3.5. In some embodiments of any of the aspects, the anion has a Log P of about 2.8 to about 3.01.

In some embodiments of any of the aspects, the carboxylic acid comprises a carbon backbone chain having 8 carbons and has a Log P greater than are equal to 2.8 and a pKa between 4.8 and 5.2. In some embodiments of any of the aspects, the carboxylic acid has a Log P greater than or equal to 2.9 and a pKa between 4.8 and 5.1.

The pKa and Log P values for anions are known in the art and/or can be calculated by one of skill in the art. For example, PubChem and SpiderChem provide these values for various anions and chemical manufacturers typically provide them as part of the catalog listings for their products. pKa and Log P values for exemplary anions are provided in Table 3 herein.

In some embodiments of any of the aspects, the carboxylic acid comprises a carbon chain of at least 6 carbons. In some embodiments of any of the aspects, the carboxylic acid comprises a carbon chain of at least 7 carbons. In some embodiments of any of the aspects, the carboxylic acid comprises a carbon chain of at least 8 carbons. In some embodiments of any of the aspects, the carboxylic acid comprises a carbon chain of at least 9 carbons. In some embodiments of any of the aspects, the carboxylic acid comprises a carbon chain of at least 10 carbons. In some embodiments of any of the aspects, the carboxylic acid comprises a carbon chain of at least 11 carbons.

In some embodiments of any of the aspects, the anion comprises an alkane. In some embodiments of any of the aspects, the anion comprises an alkene. In some embodiments of any of the aspects, the anion comprises a single carboxyl group. In some embodiments of any of the aspects, the carbon chain of the carboxylic acid comprises one or more substituent groups. In some embodiments of any of the aspects, the carbon chain backbone of the carboxylic acid comprises one or more substituent groups, wherein each substituent group comprises at least one carbon atom. In some embodiments of any of the aspects, the carbon chain backbone of the carboxylic acid comprises one or more substituent groups, wherein at least one substituent group comprises a methyl group. In some embodiments of any of the aspects, the carbon chain backbone of the carboxylic acid comprises two substituent groups, wherein each substituent group comprises at least one carbon atom. In some embodiments of any of the aspects, the carbon chain backbone of the carboxylic acid comprises two substituent groups, wherein one substituent group comprises a methyl group. In some embodiments of any of the aspects, the carbon chain backbone of the carboxylic acid comprises two substituent groups, wherein each substituent group comprises a methyl group.

In some embodiments of any of the aspects, the anion is based on an unsubstituted alkane. In some embodiments of any of the aspects, the anion is an unsubstituted alkene. In some embodiments of any of the aspects, the carbon chain backbone of the carboxylic acid comprises one or more substituent groups. In some embodiments of any of the aspects, the carbon chain of the carboxylic acid comprises one or more substituent groups, wherein each substituent group comprises at least one carbon atom. In some embodiments of any of the aspects, the carbon chain of the carboxylic acid comprises one or more substituent groups, wherein each substituent group is alkyl, aryl, heteroalkayl, heteroaryl, alkane, or alkene. In some embodiments of any of the aspects, the carbon chain of the carboxylic acid comprises one or more substituent groups, wherein each substituent group is unsubstituted alkyl, unsubstituted aryl, unsubstituted heteroalkayl, unsubstituted heteroaryl, unsubstituted alkane, or unsubstituted alkene.

In some embodiments of any of the aspects, the carboxylic acid comprises a carbon backbone chain having 8 carbons, is optionally a mono-alkene, and optionally has two substituents. In some embodiments of any of the aspects, at least one of the substituents is a methyl group. In some embodiments of any of the aspects, both of the substituents is a methyl group. In some embodiments of any of the aspects, the carboxylic acid is selected from the group consisting of: octanoic acid; 2-octenoic acid; 3-octenoic acid; 4-octenoic acid; 5-octenoic acid; 6-octenoic acid; 7-octenoic acid; 2,2-dimethyloctanoic acid; 2,3-dimethyloctanoic acid; 2,4-dimethyloctanoic acid; 2,5-dimethyloctanoic acid; 2,6-dimethyloctanoic acid; 2,7-dimethyloctanoic acid; 3,3-dimethyloctanoic acid; 3,4-dimethyloctanoic acid; 3,5-dimethyloctanoic acid; 3,6-dimethyloctanoic acid; 3,7-dimethyloctanoic acid; 4,4-dimethyloctanoic acid; 4,5-dimethyloctanoic acid; 4,6-dimethyloctanoic acid; 4,7-dimethyloctanoic acid; 5,5-dimethyloctanoic acid; 5,6-dimethyloctanoic acid; 5,7-dimethyloctanoic acid; 6,6-dimethyloctanoic acid; 6,7-dimethyloctanoic acid; 7,7-dimethyloctanoic acid; 2,3-dimethyl-2-octenoic acid; 2,4-dimethyl-2-octenoic acid; 2,5-dimethyl-2-octenoic acid; 2,6-dimethyl-2-octenoic acid; 2,7-dimethyl-2-octenoic acid; 3,4-dimethyl-2-octenoic acid; 3,5-dimethyl-2-octenoic acid; 3,6-dimethyl-2-octenoic acid; 3,7-dimethyl-2-octenoic acid; 4,4-dimethyl-2-octenoic acid; 4,5-dimethyl-2-octenoic acid; 4,6-dimethyl-2-octenoic acid; 4,7-dimethyl-2-octenoic acid; 5,5-dimethyl-2-octenoic acid; 5,6-dimethyl-2-octenoic acid; 5,7-dimethyl-2-octenoic acid; 6,6-dimethyl-2-octenoic acid; 6,7-dimethyl-2-octenoic acid, 7,7-dimethyl-2-octenoic acid; 2,2-dimethyl-3-octenoic acid; 2,3-dimethyl-3-octenoic acid; 2,4-dimethyl-3-octenoic acid; 2,5-dimethyl-3-octenoic acid; 2,6-dimethyl-3-octenoic acid; 2,7-dimethyl-3-octenoic acid; 3,4-dimethyl-3-octenoic acid; 3,5-dimethyl-3-octenoic acid; 3,6-dimethyl-3-octenoic acid; 3,7-dimethyl-3-octenoic acid; 4,5-dimethyl-3-octenoic acid; 4,6-dimethyl-3-octenoic acid; 4,7-dimethyl-3-octenoic acid; 5,5-dimethyl-3-octenoic acid; 5,6-dimethyl-3-octenoic acid; 5,7-dimethyl-3-octenoic acid; 6,6-dimethyl-3-octenoic acid; 6,7-dimethyl-3-octenoic acid; 7,7-dimethyl-3-octenoic acid; 2,2-dimethyl-4-octenoic acid; 2,3-dimethyl-4-octenoic acid; 2,4-dimethyl-4-octenoic acid; 2,5-dimethyl-4-octenoic acid; 2,6-dimethyl-4-octenoic acid; 2,7-dimethyl-4-octenoic acid; 3,3-dimethyl-4-octenoic acid; 3,4-dimethyl-4-octenoic acid; 3,5-dimethyl-4-octenoic acid; 3,6-dimethyl-4-octenoic acid; 3,7-dimethyl-4-octenoic acid; 4,5-dimethyl-4-octenoic acid; 4,6-dimethyl-4-octenoic acid; 4,7-dimethyl-4-octenoic acid; 5,6-dimethyl-4-octenoic acid; 5,7-dimethyl-4-octenoic acid; 6,6-dimethyl-4-octenoic acid; 6,7-dimethyl-4-octenoic acid; 7,7-dimethyl-4-octenoic acid; 2,2-dimethyl-5-octenoic acid; 2,3-dimethyl-5-octenoic acid; 2,4-dimethyl-5-octenoic acid; 2,5-dimethyl-5-octenoic acid; 2,6-dimethyl-5-octenoic acid; 2,7-dimethyl-5-octenoic acid; 3,3-dimethyl-5-octenoic acid; 3,4-dimethyl-5-octenoic acid; 3,5-dimethyl-5-octenoic acid; 3,6-dimethyl-5-octenoic acid; 3,7-dimethyl-5-octenoic acid; 4,4-dimethyl-5-octenoic acid; 4,5-dimethyl-5-octenoic acid; 4,6-dimethyl-5-octenoic acid; 4,7-dimethyl-5-octenoic acid; 5,6-dimethyl-5-octenoic acid; 5,7-dimethyl-5-octenoic acid; 6,7-dimethyl-5-octenoic acid; 7,7-dimethyl-5-octenoic acid; 2,2-dimethyl-6-octenoic acid; 2,3-dimethyl-6-octenoic acid; 2,4-dimethyl-6-octenoic acid; 2,5-dimethyl-6-octenoic acid; 2,6-dimethyl-6-octenoic acid; 2,7-dimethyl-6-octenoic acid; 3,3-dimethyl-6-octenoic acid; 3,4-dimethyl-6-octenoic acid; 3,5-dimethyl-6-octenoic acid; 3,6-dimethyl-6-octenoic acid; 3,7-dimethyl-6-octenoic acid (citranellic acid); 4,4-dimethyl-6-octenoic acid; 4,5-dimethyl-6-octenoic acid; 4,6-dimethyl-6-octenoic acid; 4,7-dimethyl-6-octenoic acid; 5,5-dimethyl-6-octenoic acid; 5,6-dimethyl-6-octenoic acid; 5,7-dimethyl-6-octenoic acid; 6,7-dimethyl-6-octenoic acid; 2,2-dimethyl-7-octenoic acid; 2,3-dimethyl-7-octenoic acid; 2,4-dimethyl-7-octenoic acid; 2,5-dimethyl-7-octenoic acid; 2,6-dimethyl-7-octenoic acid; 2,7-dimethyl-7-octenoic acid; 4,4-dimethyl-7-octenoic acid; 3,4-dimethyl-7-octenoic acid; 3,5-dimethyl-7-octenoic acid; 3,6-dimethyl-7-octenoic acid; 3,7-dimethyl-7-octenoic acid; 4,4-dimethyl-7-octenoic acid; 4,5-dimethyl-7-octenoic acid; 4,6-dimethyl-7-octenoic acid; 4,7-dimethyl-7-octenoic acid; 5,5-dimethyl-7-octenoic acid; 5,6-dimethyl-7-octenoic acid; 5,7-dimethyl-7-octenoic acid; 6,6-dimethyl-7-octenoic acid; 6,7-dimethyl-7-octenoic acid; and isomers thereof. In some embodiments of any of the aspects, the carboxylic acid is selected from the group consisting of: octanoic acid; 2-octenoic acid; 3-octenoic acid; 4-octenoic acid; 5-octenoic acid; 6-octenoic acid; 7-octenoic acid; 2,2-dimethyloctanoic acid; 2,4-dimethyloctanoic acid; 2,5-dimethyloctanoic acid; 2,6-dimethyloctanoic acid; 2,7-dimethyloctanoic acid; 3,3-dimethyloctanoic acid; 3,5-dimethyloctanoic acid; 3,6-dimethyloctanoic acid; 3,7-dimethyloctanoic acid; 4,4-dimethyloctanoic acid; 4,5-dimethyloctanoic acid; 4,6-dimethyloctanoic acid; 5,5-dimethyloctanoic acid; 5,6-dimethyloctanoic acid; 5,7-dimethyloctanoic acid; 6,6-dimethyloctanoic acid; 7,7-dimethyloctanoic acid; 3,7-dimethyl-2-octenoic acid; 3,7-dimethyl-3-octenoic acid; 3,7-dimethyl-4-octenoic acid; 2,7-dimethyl-6-octenoic acid; 3,7-dimethyl-6-octenoic acid (citronellic acid); 2,2-dimethyl-7-octenoic acid; 2,3-dimethyl-7-octenoic acid; and isomers thereof. In some embodiments of any of the aspects, the carboxylic acid is selected from the group consisting of citronellic acid, octanoic acid, 2-octenoic acid and isomers thereof. In some embodiments of any of the aspects, the carboxylic acid is selected from the group consisting of citronellic acid, octanoic acid or trans-2-octenoic acid. In some embodiments of any of the aspects, octenoic acid as used herein (for example in Table 3) refers to trans-2-octenoic acid.

In some embodiments of any of the aspects, the carboxylic acid comprises a carbon backbone chain having 8 carbons and is optionally a mono-alkene. In some embodiments of any of the aspects, the carbon backbone chain of the carboxylic acid is not substituted. In some embodiments of any of the aspects, the carboxylic acid is selected from the group consisting of octanoic acid, 2-octenoic acid, 3-octenoic acid, 4-octenoic acid, 5-octenoic acid, 6-octenoic acid, 7-octenoic acid and isomers thereof. In some options, the carboxylic acid is octanoic acid or trans-2-octenoic acid (octenoic acid).

Exemplary, non-limiting anions are provided in Table 3 below.

TABLE 3

| | LogP | pKa |
|---|---|---|
| Group 1 | | |
| Geranic Acid | 2.72 | 5.26 |
| Citronellic Acid | 2.8 | 5.19 |
| Octenoic Acid | 2.9 | 5.05 |
| Decenoic Acid | 4.02 | 5.03 |
| (9Z)-octadec-9-enoic acid | 6.5 | 5.02 |
| Group 2 | | |
| Octanoic Acid | 3.01 | 4.895 |
| Decanoic Acid | 4.09 | 4.9 |
| (9Z,12Z)-octadeca-9,12-dienoic | 7.05 | 4.77 |
| acid | | |
| (R)-5-(1,2-dithiolan-3- | 2.1 | 5.10 |
| yl)pentanoic acid | | |
| Group 3 | | |
| Hexenoic Acid | 1.8 | 5.13 |
| Group 4 | | |
| Hexanoic Acid | 1.92 | 4.88 |
| 3-methylbutanoic acid | 1.2 | 4.77 |
| Nonanedioic Acid | 1.57 | 4.55 |
| Pentanoic acid | 1.39 | 4.84 |
| Group 5 | | |
| 2-hydroxyoctanoic acid | 1.8 | 4.42 |
| (E)-3-(4-hydroxy-3-methoxy- | 1.51 | 4.42 |
| phenyl)prop-2-enoic acid | | |
| Group 6 | | |
| 2-ethylhexyl sulfate | 3.10 | |
| 2-(dimethylamino)ethanol | −0.55 | 9.3 |
| Group 7 | | |
| 8-hydroxycapric acid | 2.2 | |
| 2-methylpropanoic acid | 0.73 | 4.84 |
| Ascorbic Acid | −1.85 | 4.7 |
| Butanoic acid | 0.79 | 4.82 |
| Salicylic Acid | 2.2 | 2.97 |
| Group 8 | | |
| Hydroxyl(phenyl)acetic acid | 1.2 | 3.41 |
| Glutaric Acid | −0.29 | 4.34 |
| Adipic acid | 0.08 | 4.4 |
| Group 9 | | |
| Octanoic Acid | 3.01 | 4.895 |
| Citronellic Acid | 2.8 | 5.19 |
| Octenoic Acid | 2.9 | 5.05 |
| Group 10 | | |
| Octanoic Acid | 3.01 | 4.895 |
| Octenoic Acid | 2.9 | 5.05 |

In some embodiments of any of the aspects, the anion is selected from Table 3. In some embodiments of any of the aspects, the anion is selected from Group 1 of Table 3. In some embodiments of any of the aspects, the anion is selected from Group 2 of Table 3. In some embodiments of any of the aspects, the anion is selected from Group 3 of Table 3. In some embodiments of any of the aspects, the anion is selected from Group 4 of Table 3. In some embodiments of any of the aspects, the anion is selected from Group 5 of Table 3. In some embodiments of any of the aspects, the anion is selected from Group 6 of Table 3. In some embodiments of any of the aspects, the anion is selected from Group 7 of Table 3. In some embodiments of any of the aspects, the anion is selected from Group 8 of Table 3. In some embodiments of any of the aspects, the anion is selected from Groups 1-2 of Table 3. In some embodiments of any of the aspects, the anion is selected from Groups 1-3 of Table 3. In some embodiments of any of the aspects, the anion is selected from Groups 1-4 of Table 3. In some embodiments of any of the aspects, the anion is selected from Groups 1-5 of Table 3. In some embodiments of any of the aspects, the anion is selected from Groups 1-6 of Table 3. In some embodiments of any of the aspects, the anion is selected from Groups 1-7 of Table 3. In some embodiments of any of the aspects, the anion is selected from Group 9 of Table 3. In some embodiments of any of the aspects, the anion is selected from Group 10 of Table 3. In some embodiments of any of the aspects, the anion is selected from Groups 9-10 of Table 3.

In some embodiments of any of the aspects, the anion is geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, or hexenoic acid. In some embodiments of any of the aspects, the anion is hexenoic acid.

A quarternary ammonion is a positively charged polyatomic ion of the structure NR₄⁺, each R independently being an alkyl group or an aryl group. The general term “quaternary ammonium” relates to any compound that can be regarded as derived from ammonium hydroxide or an ammonium salt by replacement of all four hydrogen atoms of the NH₄⁺ ion by organic groups. For example, the quaternary ammonium has the structure of NR₄⁺, where each R is independently selected from hydroxyl, optionally substituted C₁-C₁₀alkyl, optionally substituted C₂-C₁₀alkenyl, optionally substituted C₂-C₁₀alkynyl, optionally substituted aryl, or optionally substituted heteroaryl.

In some embodiments of any of the aspects, the cation has a molar mass equal to or greater than choline, e.g., a molar mass equal to or greater than 104.1708 g/mol. In some embodiments of any of the aspects, the cation has a molar mass greater than choline, e.g., a molar mass equal greater than 104.1708 g/mol.

In some embodiments of any of the aspects, each R group of the quaternary ammonium independently comprises an alkyl, alkane, alkene, or aryl, at least one of the R groups comprising an ester. In some embodiments of any of the aspects, each R group of the quaternary ammonium independently comprises an alkyl, alkane, or alkene, at least one of the R groups comprising an ester. In some embodiments of any of the aspects, each R group of the quaternary ammonium independently comprises an alkane or alkene, at least one of the R groups comprising an ester. In some embodiments of any of the aspects, each R group of the quaternary ammonium independently comprises an alkyl, alkane, alkene, or aryl, one of the R groups comprising an ester. In some embodiments of any of the aspects, each R group of the quaternary ammonium independently comprises an alkyl, alkane, or alkene, one of the R groups comprising an ester. In some embodiments of any of the aspects, each R group of the quaternary ammonium independently comprises an alkane or alkene, one of the R groups comprising an ester.

In some embodiments of any of the aspects, each R group of the quaternary ammonium independently comprises a carbon chain of no more than 10 carbon atoms in length, e.g., no more than 10, 11, 12, 13, 14, 15, 16, 17, 18, 19, 20, 25, or 30 carbon atoms in length. In some embodiments of any of the aspects, each R group of the quaternary ammonium independently comprises a carbon chain of no more than 12 carbon atoms in length. In some embodiments of any of the aspects, each R group of the quaternary ammonium independently comprises a carbon chain of no more than 15 carbon atoms in length. In some embodiments of any of the aspects, each R group of the quaternary ammonium independently comprises a carbon chain of no more than 20 carbon atoms in length.

In some embodiments of any of the aspects, each R group of the quaternary ammonium independently comprises a carbon chain of no more than 10 carbon atoms, e.g., no more than 10, 11, 12, 13, 14, 15, 16, 17, 18, 19, 20, 25, or 30 carbon atoms. In some embodiments of any of the aspects, each R group of the quaternary ammonium independently comprises a carbon chain of no more than 12 carbon atoms. In some embodiments of any of the aspects, each R group of the quaternary ammonium independently comprises a carbon chain of no more than 15 carbon atoms. In some embodiments of any of the aspects, each R group of the quaternary ammonium independently comprises a carbon chain of no more than 20 carbon atoms.

In some embodiments of any of the aspects, each R group of the quaternary ammonium independently comprises an alkyl group of no more than 10 carbon atoms, e.g., no more than 10, 11, 12, 13, 14, 15, 16, 17, 18, 19, 20, 25, or 30 carbon atoms, at least one of the R groups comprising an ester. In some embodiments of any of the aspects, each R group of the quaternary ammonium independently comprises an alkyl group of no more than 12 carbon atoms. In some embodiments of any of the aspects, each R group of the quaternary ammonium independently comprises an alkyl group of no more than 15 carbon atoms. In some embodiments of any of the aspects, each R group of the quaternary ammonium independently comprises an alkyl group of no more than 20 carbon atoms, at least one R group comprising an ester.

In some embodiments of any of the aspects, each R group of the quaternary ammonium independently comprises an alkane, alkene, aryl, heteroaryl, alkyl, or heteroalkyl, at least one of the R groups comprising an ester. In some embodiments of any of the aspects, each R group of the quaternary ammonium independently comprises an unsubstituted alkane, unsubstituted alkene, unsubstituted aryl, unsubstituted heteroaryl, unsubstituted alkyl, or unsubstituted heteroalkyl, at least one of the R groups comprising an ester. In some embodiments of any of the aspects, each R group of the quaternary ammonium independently comprises an unsubstituted alkane, at least one of the R groups comprising an ester. In some embodiments of any of the aspects, each R group of the quaternary ammonium independently comprises an unsubstituted alkene, at least one of the R groups comprising an ester. In some embodiments of any of the aspects, each R group of the quaternary ammonium independently comprises one or more substituent groups, at least one of the R groups comprising an ester.

In some embodiments of any of the aspects, at least one R group of the quaternary ammonium comprises a hydroxy group, at least one of the R groups comprising an ester. In some embodiments of any of the aspects, one R group of the quaternary ammonium comprises a hydroxy group, at least one of the R groups comprising an ester. In some embodiments of any of the aspects, only one R group of the quaternary ammonium comprises a hydroxy group, at least one of the R groups comprising an ester.

In some embodiments of any of the aspects, three of the R groups are methyl, and one R group of the quaternary ammonium comprises an ester, e.g., acetylcholine. In some embodiments of any of the aspects, the cation comprises, consists of, or consists essentially of acetylcholine.

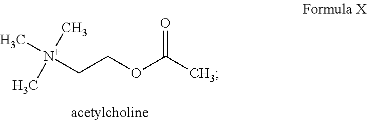

In some embodiments of any of the aspects, the cation is not choline.

Non-limiting, exemplary combinations of cation and anions are provided in Table 2 below.

TABLE 2

| | A quaternary | |
|---|---|---|
| | ammonium | |
| | comprising | |
| | an ester | |
| | group | acetylcholine |
| Glycolic acid | X | x |
| Geranic acid | X | X |
| Octenoic acid | X | X |
| Octanoic acid | X | X |
| Citronellic acid | X | X |
| Lactic acid | X | X |
| Malonic acid | X | X |
| Decenoic acid | X | X |
| Maleic acid | X | X |
| Glutaric acid | X | X |
| Citric acid | X | X |
| Decanoic acid | X | X |
| Gluconic acid | X | X |
| Adipic acid | X | X |
| (9Z,12Z)-octadeca-9,12-dienoic | X | X |
| acid | | |
| (R)-5-(1,2-dithiolan-3- | X | X |
| yl)pentanoic acid | | |
| hexenoic acid | X | x |
| Propanoic acid | X | x |

In some embodiments of any of the aspects, the ionic liquid is not CAGE (Choline And GEranate). In some embodiments of any of the aspects, the cation of the ionic liquid is not choline. In some embodiments of any of the aspects, the anion of the ionic liquid is not geranate or geranic acid.

In some embodiments of any of the aspects, the composition comprises a first ionic liquid and at least a second ionic liquid. Combinations of two, three, four, five, or more of any of the ionic liquids described herein are contemplated.

In some embodiments of any of the aspects wherein the composition comprises two or more ionic liquids, a first and second ionic liquid have the same cation, e.g., acetylcholine. In some embodiments of any of the aspects wherein the composition comprises two or more ionic liquids, a first and second ionic liquid have different anions. For example, a first ionic liquid and a second ionic liquid can each comprise a different anion selected from: lactic acid; glycolic acid; malonic acid; maleic acid; glutaric acid; citric acid; gluconic acid; adipic acid; geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, and hexenoic acid. In some embodiments, a first ionic liquid and a second ionic liquid can each comprise a different anion selected from: propanoic acid; lactic acid; glycolic acid; malonic acid; maleic acid; glutaric acid; citric acid; gluconic acid; adipic acid; geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, and hexenoic acid. In some embodiments of any of the aspects wherein the composition comprises two or more ionic liquids, the first ionic liquid has a maleic acid anion and the second ionic liquid has a hexenoic acid anion. In some embodiments of any of the aspects wherein the composition comprises two or more ionic liquids, the first ionic liquid has a propanoic acid anion and the second ionic liquid has a hexenoic acid anion. In some embodiments of any of the aspects wherein the composition comprises two or more ionic liquids, the first ionic liquid has a maleic acid anion and the second ionic liquid has a propanoic acid anion.

By way of non-limiting example, the combination of an ionic liquid comprising a hexenoic acid anion and an ionic liquid comprising a maleic acid anion is contemplated herein. By way of further non-limiting example, the combination of an ionic liquid comprising a propanoic acid anion and an ionic liquid comprising a maleic acid anion is contemplated herein. By way of further non-limiting example, the combination of an ionic liquid comprising a propanoic acid anion and an ionic liquid comprising a propanoic acid anion is contemplated herein.

By way of non-limiting example, the combination of acetylcholine hexenoic acid and acetylcholine maleic acid is contemplated herein. By way of further non-limiting example, the combination of acetylcholine propanoic acid and acetylcholine maleic acid is contemplated herein. By way of further non-limiting example, the combination of acetylcholine propanoic acid and acetylcholine propanoic acid is contemplated herein.

In some embodiments of any of the aspects comprising two or more ionic liquids, a first ionic liquid is not CAGE (Choline And GEranate). In some embodiments of any of the aspects comprising two or more ionic liquids, the cation of a first ionic liquid is not choline. In some embodiments of any of the aspects comprising two or more ionic liquids, the anion of a first ionic liquid is not geranate or geranic acid.

In some embodiments of any of the aspects wherein the composition comprises two or more ionic liquids, the second ionic liquid is choline and geranic acid (CAGE). In some embodiments of any of the aspects wherein the composition comprises two or more ionic liquids, the first ionic liquid is acetylcholine and hexenoic acid and the second ionic liquid is CAGE. In some embodiments of any of the aspects wherein the composition comprises two or more ionic liquids, the first ionic liquid is acetylcholine and maleic acid and the second ionic liquid is CAGE. In some embodiments of any of the aspects wherein the composition comprises two or more ionic liquids, the first ionic liquid is acetylcholine and propanoic acid and the second ionic liquid is CAGE.

In some embodiments of any of the aspects, the IL is at a concentration of at least 0.01% w/v. In some embodiments of any of the aspects, the IL is at a concentration of at least 0.05% w/v. In some embodiments of any of the aspects, the IL is at a concentration of at least 0.1% w/v. In some embodiments of any of the aspects, the IL is at a concentration of at least 0.2% w/v, at least 0.3% w/v, at least 0.4% w/v, at least 0.5% w/v, at least 1% w/v or greater. In some embodiments of any of the aspects, the IL is at a concentration of from about 0.01% w/v to about 1% w/v. In some embodiments of any of the aspects, the IL is at a concentration of from 0.01% w/v to 1% w/v. In some embodiments of any of the aspects, the IL is at a concentration of from about 0.05% w/v to about 0.5% w/v. In some embodiments of any of the aspects, the IL is at a concentration of from 0.05% w/v to 0.5% w/v.

In some embodiments of any of the aspects, the IL is at a concentration of less than 10% w/v. In some embodiments of any of the aspects, the IL is at a concentration of from 0.01% w/v to 10% w/v. In some embodiments of any of the aspects, the IL is at a concentration of from 0.05% w/v to 10% w/v. In some embodiments of any of the aspects, the IL is at a concentration of from 0.5% w/v to 10% w/v. In some embodiments of any of the aspects, the IL is at a concentration of from 1% w/v to 10% w/v.

In some embodiments of any of the aspects, the IL is at a concentration of at least 25% w/w. In some embodiments of any of the aspects, the IL is at a concentration of at least 25% w/w in water. In some embodiments of any of the aspects, the IL is at a concentration of at least 25% w/w in saline or a physiologically compatible buffer.

In some embodiments of any of the aspects, the IL is at a concentration of from about 5% w/w to about 75% w/w. In some embodiments of any of the aspects, the IL is at a concentration of from 5% w/w to 75% w/w. In some embodiments of any of the aspects, the IL is at a concentration of from about 5% w/w to about 75% w/w in water, saline or a physiologically compatible buffer. In some embodiments of any of the aspects, the IL is at a concentration of from 5% w/w to 75% w/w in water, saline or a physiologically compatible buffer.

In some embodiments of any of the aspects, the IL is at a concentration of at least about 0.1% w/w. In some embodiments of any of the aspects, the IL is at a concentration of at least 0.1% w/w. In some embodiments of any of the aspects, the IL is at a concentration of from about 10% w/w to about 70% w/w. In some embodiments of any of the aspects, the IL is at a concentration of from 10% w/w to 70% w/w. In some embodiments of any of the aspects, the IL is at a concentration of from about 30% w/w to about 50% w/w. In some embodiments of any of the aspects, the IL is at a concentration of from 30% w/w to 40% w/w. In some embodiments of any of the aspects, the IL is at a concentration of from about 30% w/w to about 50% w/w. In some embodiments of any of the aspects, the IL is at a concentration of from 30% w/w to 40% w/w.

In some embodiments of any of the aspects, the % w/w concentration of the IL is % w/w concentration in water, saline, or a physiologically compatible buffer.

In some embodiments of any of the aspects, the IL is 100% by w/w or w/v.

In some embodiments, the IL is an anhydrous salt, e.g., an ionic liquid not diluted or dissolved in water. In some embodiments, the IL is provided as an aqueous solution.

In some embodiments of any of the aspects, the IL is at a concentration of at least 25% w/w and has a ratio of cation:anion of at least 1:3. In some embodiments of any of the aspects, the IL is at a concentration of at least 25% w/w in water and has a ratio of cation:anion of at least 1:3. In some embodiments of any of the aspects, the IL is at a concentration of at least 25% w/w and has a ratio of cation:anion of 1:3 or 1:4. In some embodiments of any of the aspects, the IL is at a concentration of at least 25% w/w in water and has a ratio of cation:anion of 1:3 or 1:4. In some embodiments of any of the aspects, the IL is a gel, or a shear-thining Newtonian gel.

In some embodiments of any of the aspects, the IL has a ratio of cation:anion of from about 10:1 to about 1:10. In some embodiments of any of the aspects, the IL has a ratio of cation:anion of from 10:1 to 1:10. In some embodiments of any of the aspects, the IL has a ratio of cation:anion of from about 5:1 to about 1:5. In some embodiments of any of the aspects, the IL has a ratio of cation:anion of from 5:1 to 1:5. In some embodiments of any of the aspects, the IL has a ratio of cation:anion of from about 2:1 to about 1:4. In some embodiments of any of the aspects, the IL has a ratio of cation:anion of from 2:1 to 1:4. In some embodiments of any of the aspects, the IL has a ratio of cation:anion of from about 2:1 to about 1:10. In some embodiments of any of the aspects, the IL has a ratio of cation:anion of from about 2:1 to about 1:1. In some embodiments of any of the aspects, the IL has a ratio of cation:anion of from 2:1 to 1:10. In some embodiments of any of the aspects, the IL has a ratio of cation:anion of from 2:1 to 1:1. In some embodiments of any of the aspects, the IL has a ratio of cation:anion such that there is a greater amount of anion, e.g., a ratio of less than 1:1. In some embodiments of any of the aspects, the IL has a ratio of cation:anion such that there is an excess of anion. In some embodiments of any of the aspects, the IL has a ratio of cation:anion of from about 1:1 to about 1:10. In some embodiments of any of the aspects, the IL has a ratio of cation:anion of from 1:1 to 1:10. In some embodiments of any of the aspects, the IL has a ratio of cation:anion of from about 1:1 to about 1:4. In some embodiments of any of the aspects, the IL has a ratio of cation:anion of from 1:1 to 1:4. In some embodiments of any of the aspects, the IL has a ratio of cation:anion of from about 1:1 to about 1:3. In some embodiments of any of the aspects, the IL has a ratio of cation:anion of from 1:1 to 1:3. In some embodiments of any of the aspects, the IL has a ratio of cation:anion of from about 1:1 to about 1:2. In some embodiments of any of the aspects, the IL has a ratio of cation:anion of from 1:1 to 1:2. In some embodiments of any of the aspects, the IL has a ratio of cation:anion of about 1:1, 1:2, 1:3, or 1:4. In some embodiments of any of the aspects, the IL has a ratio of cation:anion of 1:1, 1:2, 1:3, or 1:4. In some embodiments of any of the aspects, the IL has a ratio of cation:anion less than about of 1:1. In some embodiments of any of the aspects, the IL has a ratio of cation:anion less than 1:1. Without wishing to be constrained by theory, compositions with higher amounts of anion relative to cation display greater hydrophobicity.

In some embodiments of any of the aspects, the IL has a cation:anion ratio with an excess of cation.

In some embodiments of any of the aspects, e.g., when one or more nucleic acid molecules are provided in combination with the IL, the ratio of cation:anion is greater than 1:1, e.g., greater than 1:2, from about 1:2 to about 1:4, or from 1:2 to 1:4.

In some embodiments of any of the aspects, the IL is at a concentration of at least 20 mM. In some embodiments of any of the aspects, the IL is at a concentration of at least about 20 mM. In some embodiments of any of the aspects, the IL is at a concentration of at least 25 mM. In some embodiments of any of the aspects, the IL is at a concentration of at least about 25 mM. In some embodiments of any of the aspects, the IL is at a concentration of at least 50 mM. In some embodiments of any of the aspects, the IL is at a concentration of at least about 50 mM. In some embodiments of any of the aspects, the IL is at a concentration of at least 100 mM, 500 mM, 1 M, 2 M, 3 M or greater. In some embodiments of any of the aspects, the IL is at a concentration of at least about 100 mM, 500 mM, 1 M, 2 M, 3 M or greater.

In some embodiments of any of the aspects, the IL is at a concentration of from about 50 mM to about 4 M. In some embodiments of any of the aspects, the IL is at a concentration of from 50 mM to 4 M. In some embodiments of any of the aspects, the IL is at a concentration of from about 500 mM to about 4 M. In some embodiments of any of the aspects, the IL is at a concentration of from 500 mM to 4 M. In some embodiments of any of the aspects, the IL is at a concentration of from about 1 M to about 4 M. In some embodiments of any of the aspects, the IL is at a concentration of from 1 M to 4 M. In some embodiments of any of the aspects, the IL is at a concentration of from about 2 M to about 4 M. In some embodiments of any of the aspects, the IL is at a concentration of from 2M to 4M.

In some embodiments of any of the aspects, the IL concentration in the composition or formulation is about 0.1 mM to 20 mM. In some embodiments of any of the aspects, the IL concentration in the composition or formulation is about 0.5 mM to 20 mM, 0.5 mM to 18 mM, 0.5 mM to 16 mM, 0.5 mM to 14 mM, 0.5 mM to 12 mM, 0.5 mM to 10 mM, 0.5 mM to 8 mM, 1 mM to 20 mM, 1 mM to 18 mM, 1 mM to 16 mM, 1 mM to 14 mM, 1 mM to 12 mM, 1 mM to 10 mM, 1 mM to 8 mM, 2 mM to 20 mM, 2 mM to 18 mM, 2 mM to 16 mM, 2 mM to 14 mM, 2 mM to 12 mM, 2 mM to 10 mM, 2 mM to 8 mM, 4 mM to 20 mM, 4 mM to 18 mM, 4 mM to 16 mM, 4 mM to 14 mM, 4 mM to 12 mM, 4 mM to 10 mM, 4 mM to 8 mM, 6 mM to 20 mM, 6 mM to 18 mM, 6 mM to 14 mM, 6 mM to 12 mM, 6 mM to 10 mM, 6 mM to 8 mM, 8 mM to 20 mM, 8 mM to 18 mM, 8 mM to 16 mM, 8 mM to 14 mM, 8 mM to 12 mM, 8 mM to 10 mM, 10 mM to 20 mM, 10 mM to 18 mM, 10 mM to 16 mM, 10 mM to 14 mM, 10 mM to 12 mM, 12 mM to 20 mM, 12 mM to 18 mM, 12 mM to 16 mM, 12 mM to 14 mM, 14 mM to 20 mM, 14 mM to 18 mM, 14 mM to 16 mM, 16 mM to 20 mM, 16 mM to 18 mM, or 18 mM to 20 mM. In some embodiments of any of the aspects, the IL concentration in the composition or formulation is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM or about 20 mM.

It is specifically contemplated that a composition or combination described herein can comprise one, two, three, or more of any of the types of components described herein. For example, a composition can comprise a mixture, solution, combination, or emulsion of two or more different ionic liquids (e.g., different ionic liquids described herein), and/or a mixture, solution, combination, or emulsion of two or more different non-ionic surfactants, and/or a mixture, solution, combination, or emulsion of two or more different active compounds.

In some embodiments of any of the aspects, the one or more ILs can be in combination with at least one compound. As used herein, “in combination with” refers to two or more substances being present in the same formulation in any molecular or physical arrangement, e.g., in an admixture, in a solution, in a mixture, in a suspension, in a colloid, in an emulsion. The formulation can be a homogeneous or heterogenous mixture. In some embodiments of any of the aspects, the active compound(s) can be comprised by a superstructure, e.g., nanoparticles, liposomes, vectors, cells, scaffolds, or the like, said superstructure is which in solution, mixture, admixture, suspension, etc., with the IL.

As used herein, an “active compound” or “active agent” is any agent which will exert an effect on a target cell or organism. The terms “compound” and “agent” refer to any entity which is normally not present or not present at the levels being administered and/or provided to a cell, tissue or subject. An agent can be selected from a group comprising: chemicals; small organic or inorganic molecules; signaling molecules; nucleic acid sequences; nucleic acid analogues; proteins; peptides; enzymes; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the agent is any chemical, entity or moiety, including without limitation synthetic and naturally-occurring non-proteinaceous entities. Agents can be known to have a desired activity and/or property, or can be selected from a library of diverse compounds. Non-limiting examples of active compounds contemplated for use in the methods described herein include small molecules, polypeptides, nucleic acids, chemotherapies/chemotherapeutic compounds, antibodies, antibody reagents, vaccines, a GLP-1 polypeptide or mimetic/analog thereof, insulin, acarbose, rituximab, or ruxolitinib.

A nucleic acid molecule, as described herein, can be a vector, an expression vector, an inhibitory nucleic acid, an aptamer, a template molecule or cassette (e.g., for gene editing), or a targeting molecule (e.g., for CRISPR-Cas technologies), or any other nucleic acid molecule that one wishes to deliver to a cell. The nucleic acid molecule can be RNA, DNA, or synthetic or modified versions thereof. In some embodiments of any of the aspects, the nucleic acid is an inhibitory nucleic acid, e.g., a siRNA. In some embodiments of any of the aspects, the nucleic acid is a siRNA, pDNA, or mRNA. In some embodiments of any of the aspects, the nucleic acid is a robed nucleic acid.

In one aspect of any of the embodiments, described herein is a method of delivering a nucleic acid molecule to a cell, the method comprising contacting the cell with the nucleic acid molecule in combination with one or more ILs as described herein. In some embodiments of any of the aspects, the cell is a cell in a subject and the contacting step comprises administering the nucleic acid molecule in combination with the one or more ILs to the subject. In some embodiments of any of the aspects, the cell is in vitro, in vivo, or ex vivo. In some embodiments of any of the aspects, the cell is eukaryotic. In some embodiments of any of the aspects, the cell is mammalian. In some embodiments of any of the aspects, the cell is an epithelial cell, e.g., an intestinal epithelial cell. In some embodiments of any of the aspects the cell is an epidermal cell.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In some embodiments of any of the aspects, the active compound can be a therapeutic compound or drug, e.g., an agent or compound which is therapeutically effective for the treatment of at least one condition in a subject. Therapeutic compounds are known in the art for a variety of conditions, see, e.g., the database available on the world wide web at drugs.com or the catalog of FDA-approved compounds available on the world wide web at catalog.data.gov/dataset/drugsfda-database; each of which is incorporated by reference herein in its entirety.

By way of non-limiting example, exemplary antibodies and/or antibody reagents suitable for use as active compounds/therapeutic compounds herein include: abciximab; adalimumab; adlimumab-atto; ado-trastuzumab; ado-trastuzumab emtansine; alemtuzumab; alirocumab; atezolizumab; avelumab; basiliximab; belimumab; bevacizumab; bezlotoxumab; blinatumomab; brentuximab; brentuximab vedotin; brodalumab; canakinumab; capromab; capromab pendetide; certolizumab; certolizumab pegol; cetuximab; daclizumab; daratumumab; denosumab; dinutuximab; dupilumab; durvalumab; eculizumab; elotuzumab; evolocumab; etanercept; etanercept-szzs; golimumab; ibritumomab; ibritumomab tiuxetan; idarucizumab; infliximab; infliximab-abda; infliximab-dyyb; ipilimumab; ixekizumab; mepolizumab; natalizumab; necitumumab; nivolumab; obiltoxaximab; obinutuzumab; ocrelizumab; ofatumumab; olaratumab; omalizumab; palivizumab; panitumumab; pembrolizumab; pertuzumab; ramucriumab; ranibizumab; raxibacumab; reslizumab; rituximab; secukinumab; siltuximab; tocilizumab; trastuzumab; ustekinumab; vedolizumab; sarilumab; guselkumab; inotuzumab ozogamicin; inotuzumab; adalimumab-adbm, gemtuzumab ozogamicin; gemtuzumab; bevacizumab-awwb; benralizumab; emicizumab; emicizumab-kxwh; trastuzumab-dkst; infliximab-qbtx; ibalizumab; ibalizumab-uiyk; tildrakizumab; tildrakizumab-asmn; burosumab; burosumab-twza; erenumab; erenumab-aooe; tositumomab; mogamulizumab; moxetumomab; moxetumomab pasudotox; cemiplimab; polatuzumab; catumaxomab; polatuzumab vedotin; and combinations thereof, including bispecific antibodies made by combining portions of the foregoing.

By way of non-limiting example, exemplary inhibitory nucleic acids suitable for use as active compounds/therapeutic compounds herein include: patisiran; and combinations thereof, including bispecific antibodies made by combining portions of the foregoing.

As used herein the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule.

In some embodiments of any of the aspects, the active compound is a hydrophobic molecule, e.g., estradiol, testosterone, corticosterone, paclitaxel, doxorubicin, cisplatin, and/or camptothecin. In some embodiments of any of the aspects, the active compound is a hydrophilic molecule.

In some embodiments of any of the aspects, the active compound is a polypeptide. In some embodiments of any of the aspects, the active compound is an antibody or antibody reagent. As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

In some embodiments of any of the aspects, the active compound has a molecular weight of greater than about 450. In some embodiments of any of the aspects, the active compound has a molecular weight of greater than about 500. In some embodiments of any of the aspects, the active compound has a molecular weight of greater than 450, e.g., greater than 450, greater than 500, greater than 550, greater than 600, greater than 1000 or more. In some embodiments of any of the aspects, the active compound is polar.

In some embodiments, the inhibitory nucleic acid is a NFKBIZ inhibitory nucleic acid, e.g., it binds to a NFKBIZ mRNA and inhibits the expression of NFKBIZ. As used herein, “NFKBIZ” or “NFKB inhibitor zeta” refers to an inhibitor of nuclear factor κB (IκB) protein IκBζ, that plays a key role in the regulation of NF-κB complexes. It is a direct transcription activator of TNF-α-, IL-17A-, and IL-36-inducible psoriasis-related gene products that are involved in inflammatory signaling, neutrophil chemotaxis, and leukocyte activation. Accordingly, provided herein are methods of treating psoriasis, e.g., by administering a composition described herein comprising an active agent that is an inhibitor of NFKBIZ, e.g., a NFKBIZ inhibitory nucleic acid. Sequences of NFKBIZ from a number of species are known in the art, e.g., human NFKBIZ sequences are available in the NCBI database under the 64332 Gene ID (e.g., mRNAs NM_001005474.3 (SEQ ID NO: 5) and NM_031419.4 (SEQ ID NO: 6)). One of skill in the art can readily design an NFKBIZ inhibitory nucleic acid, e.g., using an automated tool as described above herein. NKFBIZ inhibitory nucleic acids are also commercially available, e.g., catalog no. J-040680-06-0050 from Dharmacon (Lafayette, CO).

In some embodiments, the inhibitory nucleic acid is a TNF-alpha inhibitory nucleic acid, e.g., it binds to a TNF-alpha mRNA and inhibits the expression of TNF-alpha. As used herein, “tumor necrosis factor alpha” or “TNF-alpha” refers to a pro-inflammatory cytokine implicated in autoimmune disease, psoriasis, and other conditions. Accordingly, provided herein are methods of treating inflammatory conditions (e.g., psoriasis) and/or reducing or inhibition inflammation, e.g., by administering a composition described herein comprising an active agent that is an inhibitor of TNF-alpha, e.g., a TNF-alpha inhibitory nucleic acid. Sequences of TNF-alpha from a number of species are known in the art, e.g., human TNF-alpha sequences are available in the NCBI database under the 7124 Gene ID (e.g., mRNA NM_000594.4 (SEQ ID NO: 7)). One of skill in the art can readily design a TNF-alpha inhibitory nucleic acid, e.g., using an automated tool as described above herein. TNF-alpha inhibitory nucleic acids are also commercially available, e.g., catalog nos. J-010546-09-0002, J-010546-10-0002, J-010546-11-0002, and J-010546-12-0002, from Dharmacon (Lafayette, CO).

In some embodiments, the inhibitory nucleic acid is an IL-17 inhibitory nucleic acid, e.g., it binds to an IL-17 mRNA and inhibits the expression of IL-17. As used herein, “interleukin 17” or “IL-17” refers to a pro-inflammatory cytokine produced by activating T cells implicated in autoimmune disease, psoriasis, rheumatoid arthritis, multiple sclerosis, and other conditions. Accordingly, provided herein are methods of treating inflammatory conditions (e.g., psoriasis) and/or reducing or inhibition inflammation, e.g., by administering a composition described herein comprising an active agent that is an inhibitor of IL-17, e.g., an IL-17 inhibitory nucleic acid. Sequences of IL-17 from a number of species are known in the art, e.g., human IL-17 sequences are available in the NCBI database under the 3605 Gene ID (e.g., mRNA NM_002190.3 (SEQ ID NO: 8)). One of skill in the art can readily design an IL-17 inhibitory nucleic acid, e.g., using an automated tool as described above herein. IL-17 inhibitory nucleic acids are also commercially available, e.g., catalog no. J-007937-05-0002, J-007937-06-0002, J-007937-07-0002, and J-007937-08-0002 from Dharmacon (Lafayette, CO).

| | |
|---|---|
| SEQ ID NO: 5 | ctcctcttgc cacgaggtca gacggcgagt tcttagagaa aaaggctgct tagctgctgc |
| | ttatcatgta acctcaaaag gaaactgatc gtctttctca tgctgtcacg tacttgggtt |
| | attatcgctg attacagctg gaaacaattg atttgctctt acgtatttgt gtgacttgac |
| | tcttcaaaca caaaggttaa caggaagatc tcgagggccc tggctgaact tcaccttttg |
| | gctttcttgg cctgatgctg aactctcgag gttgagcccc atatgggggt tggcaggcag |
| | cagagaggcc cctttcaagg tgttcgggta aagaactcag tgaaggaact cctgttgcac |
| | atccgaagtc ataaacagaa ggcttctggc caagctgtgg atgattttaa gacacaaggt |
| | gtgaacatag aacagttcag agaattgaag aacacagtat catacagtgg gaaaaggaaa |
| | gggcccgatt cgttgtctga tggacctgct tgcaaaaggc cagctctgtt gcattcccaa |
| | tttttgacac cacctcaaac accaacgccc ggggagagca tggaagatgt tcatctcaat |
| | gaacccaaac aggagagcag tgctgatctg cttcagaaca ttatcaacat taagaatgaa |
| | tgcagccccg tttccctgaa cacagttcaa gttagctggc tgaaccccgt ggtggtccct |
| | cagagctccc ccgcagagca gtgtcaggac ttccatggag ggcaggtctt ttctccacct |
| | cagaaatgcc aaccattcca agtcaggggc tcccaacaaa tgatagacca ggcttccctg |
| | taccagtatt ctccacagaa ccagcatgta gagcagcagc cacactacac ccacaaacca |
| | actctggaat acagtccttt tcccatacct ccccagtccc ccgcttatga accaaacctc |
| | tttgatggtc cagaatcaca gttttgccca aaccaaagct tagtttccct tcttggtgat |
| | caaagggaat ctgagaatat tgctaatccc atgcagactt cctccagtgt tcagcagcaa |
| | aatgatgctc acttgcacag cttcagcatg atgcccagca gcgcctgtga ggccatggtg |
| | gggcacgaga tggcctctga ctcttcaaac acttcactgc cattctcaaa catgggaaat |
| | ccaatgaaca ccacacagtt agggaaatca ctttttcagt ggcaggtgga gcaggaagaa |
| | agcaaattgg caaatatttc ccaagaccag tttctttcaa aggatgcaga tggtgacacg |
| | ttccttcata ttgctgttgc ccaagggaga agggcacttt cctatgttct tgcaagaaag |
| | atgaatgcac ttcacatgct ggatattaaa gagcacaatg gacagagtgc ctttcaggtg |
| | gcagtggctg ccaatcagca tctcattgtg caggatctgg tgaacatcgg ggcacaggtg |
| | aacaccacag actgctgggg aagaacacct ctgcatgtgt gtgctgagaa gggccactcc |
| | caggtgcttc aggcgattca gaagggagca gtgggaagta atcagtttgt ggatcttgag |
| | gcaactaact atgatggcct gactcccctt cactgtgcag tcatagccca caatgctgtg |
| | gtccatgaac tccagagaaa tcaacagcct cattcacctg aagttcagga gcttttactg |
| | aagaataaga gtctggttga taccattaag tgcctaattc aaatgggagc agcggtggaa |
| | gcgaaggatc gcaaaagtgg ccgcacagcc ctgcatttgg cagctgaaga agcaaatctg |
| | gaactcattc gcctcttttt ggagctgccc agttgcctgt cttttgtgaa tgcaaaggct |
| | tacaatggca acactgccct ccatgttgct gccagcttgc agtatcggtt gacacaatta |
| | gatgctgtcc gcctgttgat gaggaaggga gcagacccaa gtactcggaa cttggagaac |
| | gaacagccag tgcatttggt tcccgatggc cctgtgggag aacagatccg acgtatcctg |
| | aagggaaagt ccattcagca gagagctcca ccgtattagc tccattagct tggagcctgg |
| | ctagcaacac tcactgtcag ttaggcagtc ctgatgtatc tgtacataga ccatttgcct |
| | tatattggca aatgtaagtt gtttctatga aacaaacata tttagttcac tattatatag |
| | tgggttatat taaaagaaaa gaagaaaaat atctaatttc tcttggcaga tttgcatatt |
| | tcatacccag gtatctggga tctagacatc tgaatttgat ctcaatggta acattgcctt |
| | caattaacag tagcttttga gtaggaaagg actttgattt gtggcacaaa acattattaa |
| | tatagctatt gacagtttca aagcaggtaa attgtaaatg tttctttaag aaaaagcatg |
| | tgaaaggaaa aaggtaaata cagcattgag gcttcatttg gccttagtcc ctgggagtta |
| | ctggcgttgg acaggcttca gtcattggac tagatgaaag gtgtccatgg ttagaatttg |
| | atctttgcaa actgtatata attgttattt ttgtccttaa aaatattgta catacttggt |
| | tgttaacatg gtcatatttg aaatgtataa gtccataaaa tagaaaagaa caagtgaatt |
| | gttgctattt aaaaaaattt tacaattctt actaaggagt ttttattgtg taatcactaa |
| | gtctttgtag ataaagcaga tggggagtta cggagttgtt cctttactgg ctgaaagata |
| | tattcgaatt gtaaagatgc tttttctcat gcattgaaat tatacattat ttgtagggaa |
| | ttgcatgctt tttttttttt ttctcccgag acagggtctt gctctggcgc ccaggctgga |
| | gtacagtggc atgatcttgg ctcacttcag ccttgacttg ggctcaagtg atcctcctac |
| | ctgagccttc tgagtaactg gaactacagg tgtgcactcc tcgcctggct aattttttat |
| | tttttgtaca ggcaggatct tgccaccttg cccaggctgg tcttgaactc ctgagctcat |
| | gccatctgcc tgccttagtc tcccaaaatg ctgggattac aggagtgagc caccatgccc |
| | ggctggcagt tgcatggaag agaacacctc tttatggctt accctctaga atttctaatt |
| | tatgtgttct gttgaaattt ttgttttttt acctttattg aaacaacaaa aagtcagtat |
| | tgaaacatat cttcctgttt tctgttgtca aatgatgata atgtgccatg atgttttata |
| | tatatcattc agaaaaagtt ttatttttta ataacattct attaacatta ttttgcttgc |
| | cgctggcatg cctgaggaat gtatttggct ttgattacac actaagtttt tgtaataaat |
| | ttgactcatt aaaaaccttt tttttttaaa aaaaaaaaaa aagaaaatct cattagtgaa |
| | cttatctttg cagctgagta cttaaattct ttttaaaaag ataccctttg gattgatcac |
| | attgtttgac ccagtatgtc ttgtagacac gttagttata atcaccttgt atctctaaat |
| | atggtgtgat atgaaccagt ccattcacat tggaaaaact gatggtttta aataaactaa |
| | ttcactaata |
| SEQ ID NO: 6 | gtactggccc gcgccgtccg cccgccgaca gctccctgag ccagcccggg aggcagccgc |
| | gcgcagcgag ccggtggcgc aggtgtcggg gtcctcgagc gcccagcctg ggagcatgat |
| | tgtggacaag ctgctggacg acagccgcgg cggagagggg ctgcgggacg cggcgggcgg |
| | ctgcggcctc atgaccagcc cgctcaacct gagctacttc tacggcgcgt cgccgcccgc |
| | cgccgccccg ggcgcctgcg acgccagctg ctcggtcttg ggcccctcgg cgcccggctc |
| | gcccggctcc gactcctccg acttctcctc tgcctcgtcg gtgtcctcct gcggcgccgt |
| | ggagtcccgg tcgagaggcg gcgcccgcgc cgagcgccag ccagttgagc cccatatggg |
| | ggttggcagg cagcagagag gcccctttca aggtgttcgg gtaaagaact cagtgaagga |
| | actcctgttg cacatccgaa gtcataaaca gaaggcttct ggccaagctg tggatgattt |
| | taagacacaa ggtgtgaaca tagaacagtt cagagaattg aagaacacag tatcatacag |
| | tgggaaaagg aaagggcccg attcgttgtc tgatggacct gcttgcaaaa ggccagctct |
| | gttgcattcc caatttttga caccacctca aacaccaacg cccggggaga gcatggaaga |
| | tgttcatctc aatgaaccca aacaggagag cagtgctgat ctgcttcaga acattatcaa |
| | cattaagaat gaatgcagcc ccgtttccct gaacacagtt caagttagct ggctgaaccc |
| | cgtggtggtc cctcagagct cccccgcaga gcagtgtcag gacttccatg gagggcaggt |
| | cttttctcca cctcagaaat gccaaccatt ccaagtcagg ggctcccaac aaatgataga |
| | ccaggcttcc ctgtaccagt attctccaca gaaccagcat gtagagcagc agccacacta |
| | cacccacaaa ccaactctgg aatacagtcc ttttcccata cctccccagt cccccgctta |
| | tgaaccaaac ctctttgatg gtccagaatc acagttttgc ccaaaccaaa gcttagtttc |
| | ccttcttggt gatcaaaggg aatctgagaa tattgctaat cccatgcaga cttcctccag |
| | tgttcagcag caaaatgatg ctcacttgca cagcttcagc atgatgccca gcagcgcctg |
| | tgaggccatg gtggggcacg agatggcctc tgactcttca aacacttcac tgccattctc |
| | aaacatggga aatccaatga acaccacaca gttagggaaa tcactttttc agtggcaggt |
| | ggagcaggaa gaaagcaaat tggcaaatat ttcccaagac cagtttcttt caaaggatgc |
| | agatggtgac acgttccttc atattgctgt tgcccaaggg agaagggcac tttcctatgt |
| | tcttgcaaga aagatgaatg cacttcacat gctggatatt aaagagcaca atggacagag |
| | tgcctttcag gtggcagtgg ctgccaatca gcatctcatt gtgcaggatc tggtgaacat |
| | cggggcacag gtgaacacca cagactgctg gggaagaaca cctctgcatg tgtgtgctga |
| | gaagggccac tcccaggtgc ttcaggcgat tcagaaggga gcagtgggaa gtaatcagtt |
| | tgtggatctt gaggcaacta actatgatgg cctgactccc cttcactgtg cagtcatagc |
| | ccacaatgct gtggtccatg aactccagag aaatcaacag cctcattcac ctgaagttca |
| | ggagctttta ctgaagaata agagtctggt tgataccatt aagtgcctaa ttcaaatggg |
| | agcagcggtg gaagcgaagg atcgcaaaag tggccgcaca gccctgcatt tggcagctga |
| | agaagcaaat ctggaactca ttcgcctctt tttggagctg cccagttgcc tgtcttttgt |
| | gaatgcaaag gcttacaatg gcaacactgc cctccatgtt gctgccagct tgcagtatcg |
| | gttgacacaa ttagatgctg tccgcctgtt gatgaggaag ggagcagacc caagtactcg |
| | gaacttggag aacgaacagc cagtgcattt ggttcccgat ggccctgtgg gagaacagat |
| | ccgacgtatc ctgaagggaa agtccattca gcagagagct ccaccgtatt agctccatta |
| | gcttggagcc tggctagcaa cactcactgt cagttaggca gtcctgatgt atctgtacat |
| | agaccatttg ccttatattg gcaaatgtaa gttgtttcta tgaaacaaac atatttagtt |
| | cactattata tagtgggtta tattaaaaga aaagaagaaa aatatctaat ttctcttggc |
| | agatttgcat atttcatacc caggtatctg ggatctagac atctgaattt gatctcaatg |
| | gtaacattgc cttcaattaa cagtagcttt tgagtaggaa aggactttga tttgtggcac |
| | aaaacattat taatatagct attgacagtt tcaaagcagg taaattgtaa atgtttcttt |
| | aagaaaaagc atgtgaaagg aaaaaggtaa atacagcatt gaggcttcat ttggccttag |
| | tccctgggag ttactggcgt tggacaggct tcagtcattg gactagatga aaggtgtcca |
| | tggttagaat ttgatctttg caaactgtat ataattgtta tttttgtcct taaaaatatt |
| | gtacatactt ggttgttaac atggtcatat ttgaaatgta taagtccata aaatagaaaa |
| | gaacaagtga attgttgcta tttaaaaaaa ttttacaatt cttactaagg agtttttatt |
| | gtgtaatcac taagtctttg tagataaagc agatggggag ttacggagtt gttcctttac |
| | tggctgaaag atatattcga attgtaaaga tgctttttct catgcattga aattatacat |
| | tatttgtagg gaattgcatg cttttttttt tttttctccc gagacagggt cttgctctgg |
| | cgcccaggct ggagtacagt ggcatgatct tggctcactt cagccttgac ttgggctcaa |
| | gtgatcctcc tacctgagcc ttctgagtaa ctggaactac aggtgtgcac tcctcgcctg |
| | gctaattttt tattttttgt acaggcagga tcttgccacc ttgcccaggc tggtcttgaa |
| | ctcctgagct catgccatct gcctgcctta gtctcccaaa atgctgggat tacaggagtg |
| | agccaccatg cccggctggc agttgcatgg aagagaacac ctctttatgg cttaccctct |
| | agaatttcta atttatgtgt tctgttgaaa tttttgtttt tttaccttta ttgaaacaac |
| | aaaaagtcag tattgaaaca tatcttcctg ttttctgttg tcaaatgatg ataatgtgcc |
| | atgatgtttt atatatatca ttcagaaaaa gttttatttt ttaataacat tctattaaca |
| | ttattttgct tgccgctggc atgcctgagg aatgtatttg gctttgatta cacactaagt |
| | ttttgtaata aatttgactc attaaaaacc tttttttttt aaaaaaaaaa aaaaagaaaa |
| | tctcattagt gaacttatct ttgcagctga gtacttaaat tctttttaaa aagataccct |
| | ttggattgat cacattgttt gacccagtat gtcttgtaga cacgttagtt ataatcacct |
| | tgtatctcta aatatggtgt gatatgaacc agtccattca cattggaaaa actgatggtt |
| | ttaaataaac taattcacta ata |
| SEQ ID NO: 7 | agcagacgct ccctcagcaa ggacagcaga ggaccagcta agagggagag aagcaactac |
| | agaccccccc tgaaaacaac cctcagacgc cacatcccct gacaagctgc caggcaggtt |
| | ctcttcctct cacatactga cccacggctc caccctctct cccctggaaa ggacaccatg |
| | agcactgaaa gcatgatccg ggacgtggag ctggccgagg aggcgctccc caagaagaca |
| | ggggggcccc agggctccag gcggtgcttg ttcctcagcc tcttctcctt cctgatcgtg |
| | gcaggcgcca ccacgctctt ctgcctgctg cactttggag tgatcggccc ccagagggaa |
| | gagttcccca gggacctctc tctaatcagc cctctggccc aggcagtcag atcatcttct |
| | cgaaccccga gtgacaagcc tgtagcccat gttgtagcaa accctcaagc tgaggggcag |
| | ctccagtggc tgaaccgccg ggccaatgcc ctcctggcca atggcgtgga gctgagagat |
| | aaccagctgg tggtgccatc agagggcctg tacctcatct actcccaggt cctcttcaag |
| | ggccaaggct gcccctccac ccatgtgctc ctcacccaca ccatcagccg catcgccgtc |
| | tcctaccaga ccaaggtcaa cctcctctct gccatcaaga gcccctgcca gagggagacc |
| | ccagaggggg ctgaggccaa gccctggtat gagcccatct atctgggagg ggtcttccag |
| | ctggagaagg gtgaccgact cagcgctgag atcaatcggc ccgactatct cgactttgcc |
| | gagtctgggc aggtctactt tgggatcatt gccctgtgag gaggacgaac atccaacctt |
| | cccaaacgcc tcccctgccc caatcccttt attaccccct ccttcagaca ccctcaacct |
| | cttctggctc aaaaagagaa ttgggggctt agggtcggaa cccaagctta gaactttaag |
| | caacaagacc accacttcga aacctgggat tcaggaatgt gtggcctgca cagtgaagtg |
| | ctggcaacca ctaagaattc aaactggggc ctccagaact cactggggcc tacagctttg |
| | atccctgaca tctggaatct ggagaccagg gagcctttgg ttctggccag aatgctgcag |
| | gacttgagaa gacctcacct agaaattgac acaagtggac cttaggcctt cctctctcca |
| | gatgtttcca gacttccttg agacacggag cccagccctc cccatggagc cagctccctc |
| | tatttatgtt tgcacttgtg attatttatt atttatttat tatttattta tttacagatg |
| | aatgtattta tttgggagac cggggtatcc tgggggaccc aatgtaggag ctgccttggc |
| | tcagacatgt tttccgtgaa aacggagctg aacaataggc tgttcccatg tagccccctg |
| | gcctctgtgc cttcttttga ttatgttttt taaaatattt atctgattaa gttgtctaaa |
| | caatgctgat ttggtgacca actgtcactc attgctgagc ctctgctccc caggggagtt |
| | gtgtctgtaa tcgccctact attcagtggc gagaaataaa gtttgcttag aaaagaaa |
| SEQ ID NO: 8 | gtccatctca tagcaggcac aaactcatcc atccccagtt gattggaaga aacaacgatg |
| | actcctggga agacctcatt ggtgtcactg ctactgctgc tgagcctgga ggccatagtg |
| | aaggcaggaa tcacaatccc acgaaatcca ggatgcccaa attctgagga caagaacttc |
| | ccccggactg tgatggtcaa cctgaacatc cataaccgga ataccaatac caatcccaaa |
| | aggtcctcag attactacaa ccgatccacc tcaccttgga atctccaccg caatgaggac |
| | cctgagagat atccctctgt gatctgggag gcaaagtgcc gccacttggg ctgcatcaac |
| | gctgatggga acgtggacta ccacatgaac tctgtcccca tccagcaaga gatcctggtc |
| | ctgcgcaggg agcctccaca ctgccccaac tccttccggc tggagaagat actggtgtcc |
| | gtgggctgca cctgtgtcac cccgattgtc caccatgtgg cctaagagct ctggggagcc |
| | cacactcccc aaagcagtta gactatggag agccgaccca gcccctcagg aaccctcatc |
| | cttcaaagac agcctcattt cggactaaac tcattagagt tcttaaggca gtttgtccaa |
| | ttaaagcttc agaggtaaca cttggccaag atatgagatc tgaattacct ttccctcttt |
| | ccaagaagga aggtttgact gagtaccaat ttgcttcttg tttacttttt taagggcttt |
| | aagttattta tgtatttaat atgccctgag ataactttgg ggtataagat tccattttaa |
| | tgaattacct actttatttt gtttgtcttt ttaaagaaga taagattctg ggcttgggaa |
| | ttttattatt taaaaggtaa aacctgtatt tatttgagct atttaaggat ctatttatgt |
| | ttaagtattt agaaaaaggt gaaaaagcac tattatcagt tctgcctagg taaatgtaag |
| | atagaattaa atggcagtgc aaaatttctg agtctttaca acatacggat atagtatttc |
| | ctcctctttg tttttaaaag ttataacatg gctgaaaaga aagattaaac ctactttcat |
| | atgtattaat ttaaattttg caatttgttg aggttttaca agagatacag caagtctaac |
| | tctctgttcc attaaaccct tataataaaa tccttctgta ataataaagt ttcaaaagaa |
| | aatgtttatt tgttctcatt aaatgtattt tagcaaactc agctcttccc tattgggaag |
| | agttatgcaa attctcctat aagcaaaaca aagcatgtct ttgagtaaca atgacctgga |
| | aatacccaaa attccaagtt ctcgatttca catgccttca agactgaaca ccgactaagg |
| | ttttcatact attagccaat gctgtagaca gaagcatttt gataggaata gagcaaataa |
| | gataatggcc ctgaggaatg gcatgtcatt attaaagatc atatggggaa aatgaaaccc |
| | tccccaaaat acaagaagtt ctgggaggag acattgtctt cagactacaa tgtccagttt |
| | ctcccctaga ctcaggcttc ctttggagat taaggcccct cagagatcaa cagaccaaca |
| | tttttctctt cctcaagcaa cactcctagg gcctggcttc tgtctgatca aggcaccaca |
| | caacccagaa aggagctgat ggggcagaac gaactttaag tatgagaaaa gttcagccca |
| | agtaaaataa aaactcaatc acattcaatt ccagagtagt ttcaagtttc acatcgtaac |
| | cattttcgcc c |

In one aspect of any of the embodiments, provided herein is a method of treating an inflammatory condition and/or a method of reducing inflammation in a subject in need thereof, the method comprising administering a composition described herein, comprising at least one IL and at least one anti-inflammatory agent to the subject. In some embodiments of any of the aspects, the anti-inflammatory agent is an inhibitory nucleic acid that targets one or more pro-inflammatory gene products, e.g., IL-17, TNF-alpha, and/or NFKBIZ.

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, the inflammatory condition is an inflammatory condition of the skin. In some embodiments of the aspects, the inflammatory condition is an autoimmune disease.

Non-limiting examples of inflammatory conditions of the skin can include psoriasis, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions. By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex. By way of non-limiting example, inflammatory conditions can be inflammatory conditions of the cardiovascular system, such as coronary infarct damage, peripheral vascular disease, myocarditis, vasculitis, revascularization of stenosis, atherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia. By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection. By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-1 associated myelopathy/tropical spactic paraparesis; Devic's disease; human immunodeficiency virus encephalopathy; human immunodeficiency virus vacuolar myelopathy; peripheral neuropathies; Guillain-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS). Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/giant cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines. In some embodiments, an inflammatory condition is associated with an infection, e.g., viral, bacterial, fungal, parasite or prion infections. In some embodiments, an inflammatory condition is associated with an allergic response. In some embodiments, an inflammatory condition is associated with a pollutant (e.g., asbestosis, silicosis, or berylliosis).

In some embodiments, the inflammatory condition can be a local condition, e.g., a rash or allergic reaction. In some embodiments, the inflammation is associated with a wound.

Anti-inflammatory agents are known in the art and can include, by way of non-limiting example, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and inhibitors of pro-inflammatory gene products (e.g., inhibitory nucleic acids as described above herein). Pro-inflammatory genes are known in the art and include, by way of non-limiting example, NKFBIZ, TNF-alpha, IL-17, IL-36 (IL-37alpha, IL-36beta, and IL-36gamma), IL-22, IL-17C, CXCL8, CCL20, IL23A, DEFB4, and LCN2.

As used herein, “composition” refers to any IL, combination of ILs, or combination of one or more ILs and one or more active agents described herein, unless further specified.

In some embodiments of any of the aspects, a composition or combination as described herein, comprising at least one IL and optionally an active compound can be formulated as an oral, subcutaneous, transdermal, intratumoral, intravenous, intradermal, or parenteral formulation. In some embodiments of any of the aspects, the composition or combination as described herein can be formulated for delivery to a mucus membrane, e.g., to a nasal, oral, or vaginal membrane. In some embodiments of any of the aspects, an oral formulation can be a degradable capsule comprising the composition comprising the at least one IL and optionally, an active compound.

In some embodiments of any of the aspects, the composition or combination as described herein, comprising at least one IL and optionally an active compound can be formulated as a subcutaneous, transdermal, or topical formulation. In some embodiments of any of the aspects, the composition or combination as described herein, comprising at least one IL and a nucleic acid can be formulated as a subcutaneous, transdermal, or topical formulation. In some embodiments of any of the aspects, the composition or combination as described herein, comprising at least one IL and a nucleic acid can be formulated as a subcutaneous, transdermal, or topical formulation for use in a method of treating a dermatological disease, e.g., psoriasis, basal cell carcinoma, squamous cell carcinoma, atopic dermatitis, alopecia, or aging. In some embodiments of any of the aspects, the composition or combination as described herein, comprising at least one IL and optionally an active compound can be formulated as a topical formulation. In some embodiments of any of the aspects, the composition or combination as described herein, comprising at least one IL and a nucleic acid can be formulated as a topical formulation. In some embodiments of any of the aspects, the composition or combination as described herein, comprising at least one IL and a nucleic acid can be formulated as a topical formulation for use in a method of treating a dermatological disease, e.g., psoriasis, basal cell carcinoma, squamous cell carcinoma, atopic dermatitis, alopecia, or aging.

In some embodiments of any of the aspects, the composition or combination as described herein, comprising at least one IL and a siRNA can be formulated as a subcutaneous, transdermal, or topical formulation. In some embodiments of any of the aspects, the composition or combination as described herein, comprising at least one IL and a siRNA can be formulated as a subcutaneous, transdermal, or topical formulation for use in a method of treating a dermatological disease, e.g., psoriasis, basal cell carcinoma, squamous cell carcinoma, atopic dermatitis, alopecia, or aging. In some embodiments of any of the aspects, the composition or combination as described herein, comprising at least one IL and a siRNA can be formulated as topical formulation. In some embodiments of any of the aspects, the composition or combination as described herein, comprising at least one IL and a siRNA can be formulated as a topical formulation for use in a method of treating a dermatological disease, e.g., psoriasis, basal cell carcinoma, squamous cell carcinoma, atopic dermatitis, alopecia, or aging.

In some embodiments of any of the aspects, the composition or combination as described herein, comprising at least one IL and a pDNA can be formulated as a subcutaneous, transdermal, or topical formulation. In some embodiments of any of the aspects, the composition or combination as described herein, comprising at least one IL and a pDNA can be formulated as a subcutaneous, transdermal, or topical formulation for use in a method of treating a dermatological disease, e.g., psoriasis, basal cell carcinoma, squamous cell carcinoma, atopic dermatitis, alopecia, or aging. In some embodiments of any of the aspects, the composition or combination as described herein, comprising at least one IL and a pDNA can be formulated as a topical formulation. In some embodiments of any of the aspects, the composition or combination as described herein, comprising at least one IL and a pDNA can be formulated as a topical formulation for use in a method of treating a dermatological disease, e.g., psoriasis, basal cell carcinoma, squamous cell carcinoma, atopic dermatitis, alopecia, or aging.

In some embodiments of any of the aspects, described herein is a composition comprising at least one IL as described herein and at least one active compound. In some embodiments of any of the aspects, described herein is a composition consisting essentially of at least one IL as described herein and at least one active compound. In some embodiments of any of the aspects, described herein is a composition consisting of at least one IL as described herein and at least one active compound. In some embodiments of any of the aspects, the composition comprising at least one IL as described herein and at least one active compound is administered as a monotherapy, e.g., another treatment for the condition is not administered to the subject.

In one aspect of any of the embodiments, described herein is a pharmaceutical composition comprising at least one active compound in combination with at least one IL as described herein. In some embodiments, the pharmaceutical composition comprises the at least one IL and the one or more active compounds as described herein. In some embodiments, the pharmaceutical composition consists essentially of the at least one IL and the one or more active compounds as described herein. In some embodiments, the pharmaceutical composition consists of the at least one IL and the one or more active compounds as described herein. In some embodiments, the pharmaceutical composition consists essentially of an aqueous solution of the at least one IL and the one or more active compounds as described herein. In some embodiments, the pharmaceutical composition consists of an aqueous solution of the at least one IL and the one or more active compounds as described herein.

The compositions, formulations, and combinations described herein can comprise at least one IL as described herein, e.g., one IL, two ILs, three ILs, or more. In some embodiments of any of the aspects, a composition, formulation, or combination as described herein can comprise at least one IL as described herein and CAGE (Choline And GEranate).

In some embodiments of any of the aspects, the at least one active compound and the at least one ionic liquid are further in combination with at least one non-ionic surfactant. As used herein, “non-ionic surfactant” refers to a surfactant which lacks a net ionic charge and does not dissociate to an appreciable extent in aqueous media. The properties of non-ionic surfactants are largely dependent upon the proportions of the hydrophilic and hydrophobic groups in the molecule. Hydrophilic groups include the oxyethylene group (—OCH2CH2-) and the hydroxy group. By varying the number of these groups in a hydrophobic molecule, such as a fatty acid, substances are obtained which range from strongly hydrophobic and water insoluble compounds, such as glyceryl monostearate, to strongly hydrophilic and water-soluble compounds, such as the macrogols. Between these two extremes types include those in which the proportions of the hydrophilic and hydrophobic groups are more evenly balanced, such as the macrogol esters and ethers and sorbitan derivatives. Suitable non-ionic surfactants may be found in Martindale, The Extra Pharmacopoeia, 28th Edition, 1982, The Pharmaceutical Press, London, Great Britain, pp. 370 to 379. Non-limiting examples of non-ionic surfactants include polysorbates, a Tween™, block copolymers of ethylene oxide and propylene oxide, glycol and glyceryl esters of fatty acids and their derivatives, polyoxyethylene esters of fatty acids (macrogol esters), polyoxyethylene ethers of fatty acids and their derivatives (macrogol ethers), polyvinyl alcohols, and sorbitan esters, sorbitan monoesters, ethers formed from fatty alcohols and polyethylene glycol, polyoxyethylene-polypropylene glycol, alkyl polyglycoside, Cetomacrogol 1000, cetostearyl alcohol, cetyl alcohol, cocamide DEA, cocamide MEA, decyl glucoside, decyl polyglucose, glycerol monostearate, IGEPAL CA-630, isoceteth-20, lauryl glucoside, maltosides, monolaurin, mycosubtilin, Nonidet P-40, nonoxynol-9, nonoxynols, NP-40, octaethylene glycol monododecyl ether, N-Octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, sorbitan, sorbitan monolaurate, sorbitan monostearate, sorbitan tristearate, stearyl alcohol, surfactin, Triton X-100, and the like. In some embodiments of any of the aspects, the at least one non-ionic surfactant has a neutral hydrophilic head group.

As used herein, “polysorbate” refers to a surfactant derived from ethoxylated sorbitan (a derivative of sorbitol) esterified with fatty acids. Common brand names for polysorbates include Scattics™, Alkest™, Canarcel™, and Tween™. Exemplary polysorbates include polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and polysorbate 80 (polyoxyethylene (20) sorbitan monooleate).

In some embodiments of any of the aspects, the at least one non-ionic surfactant (e.g., at least one polysorbate) is present at a concentration of about 0.10% to about 50% w/v. In some embodiments of any of the aspects, the at least one non-ionic surfactant (e.g., at least one polysorbate) is present at a concentration of 0.1% to 50% w/v. In some embodiments of any of the aspects, the at least one non-ionic surfactant (e.g., at least one polysorbate) is present at a concentration of about 1% to about 5% w/v. In some embodiments of any of the aspects, the at least one non-ionic surfactant (e.g., at least one polysorbate) is present at a concentration of 1% to 5% w/v. In some embodiments of any of the aspects, the at least one non-ionic surfactant (e.g., at least one polysorbate) is present at a concentration of about 3% to about 10% w/v. In some embodiments of any of the aspects, the at least one non-ionic surfactant (e.g., at least one polysorbate) is present at a concentration of 3% to 10% w/v. In some embodiments of any of the aspects, the at least one non-ionic surfactant (e.g., at least one polysorbate) is present at a concentration of less than about 5% w/v. In some embodiments of any of the aspects, the at least one non-ionic surfactant (e.g., at least one polysorbate) is present at a concentration of less than 5% w/v.

In some embodiments of any of the aspects, the combination of the at least one active compound and at least one IL as described herein is provided in one or more nanoparticles. In some embodiments of any of the aspects, the combination of the at least one active compound and at least one IL as described herein comprises nanoparticles comprising the active compound, the nanoparticles in solution or suspension in a composition comprising at least one IL as described herein.

In some embodiments of any of the aspects, a composition as described herein, e.g., a composition comprising at least one IL and an active compound, can further comprise a pharmaceutically acceptable carrier. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present disclosure can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques. Suitable pharmaceutical carriers are described in Remington's Pharmaceutical Sciences, A. Osol, a standard reference text in this field of art. For example, a parenteral composition suitable for administration by injection is prepared by dissolving 1.5% by weight of active ingredient in 0.9% sodium chloride solution.

The term “carrier” in the context of a pharmaceutical carrier refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations, and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, 18th Ed., Gennaro, ed. (Mack Publishing Co., 1990). The formulation should suit the mode of administration.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active compound. The term “pharmaceutically acceptable carrier” excludes tissue culture medium.

In some embodiments of any of the aspects, a composition as described herein, e.g., a composition comprising at least one IL as described herein and an active compound, can be formulated as an oral, subcutaneous, intravenous, intradermal, or parenteral formulation. In some embodiments of any of the aspects, an oral formulation can be a degradable capsule comprising the composition described herein, e.g., a composition comprising at least one IL as described herein and an active compound.

In some embodiments of any of the aspects described herein, the biological activity of the active compound is improved or stabilized as compared to the activity in the absence of the at least one IL. In some embodiments of any of the aspects described herein, the IL greatly enhances permeation of the active compound across the skin compared to a control where the at least one IL is absent.

In one aspect of any of the embodiments, described herein is a method of administering at least active compound to a subject using a catheter wherein the catheter is coated with at least one IL as described herein. In one aspect of any of the embodiments, described herein is a method of collecting a body fluid by placing the catheter into the body wherein the catheter is coated with at least one IL as described herein.

In one aspect of any of the embodiments, the composition or combination described herein is for a method of administering or delivering at least one active compound, e.g., for the treatment of a disease. In one aspect of any of the embodiments, described herein is a method of administering at least one active compound, the method comprising administering the active compound in combination with at least one IL as described herein. In one aspect of any of the embodiments, described herein is a method of treating a disease by administering at least one active compound, the method comprising administering the active compound in combination with at least one IL as described herein.

The disease treated by the methods described herein can be, e.g., cancer (breast cancer, leukemia, lymphoma, B-cell chronic lymphocytic leukemia, glioblastoma, carcinoma, urothelial carcinoma, lung cancer, colorectal cancer, lymphoblastic leukemia, lymphocytic leukemia, sarcoma, melanoma, prostate cancer, myeloma, multiple myeloma, Non-Hodgkin's lymphoma), neuroblastoma, diabetes, an infection, inflammation, inflammatory diseases (e.g., rheumatoid arthritis, juvenile idiopathic arthritis, psoriatic arthritis, ankylosing spondylitis, crohn's disease, ulcerative colitis, plaque psoriasis), autoimmune diseases, atopic dermatitis, gastrointestinal inflammation, inflammatory bowel disease (IBD), cholesterolemia, coronary artery disease, asthma, transplant/organ rejection, systemic lupus erythematosus, multiple sclerosis, osteoporosis, and the like.

In some embodiments, the methods described herein relate to treating a subject having or diagnosed as having a condition with a composition as described herein, e.g., a comprising at least one IL and an active compound. Subjects having a condition, e.g., diabetes, can be identified by a physician using current methods of diagnosing diabetes. Symptoms and/or complications of diabetes which characterize these conditions and aid in diagnosis are well known in the art and include but are not limited to, weight loss, slow healing, polyuria, polydipsia, polyphagiam headaches, itchy skin, and fatigue. Tests that may aid in a diagnosis of, e.g. diabetes include, but are not limited to, blood tests (e.g., for fasting glucose levels). A family history of diabetes, or exposure to risk factors for diabetes (e.g. overweight) can also aid in determining if a subject is likely to have diabetes or in making a diagnosis of diabetes.

The compositions and methods described herein can be administered to a subject having or diagnosed as having a condition described herein. In some embodiments, the methods described herein comprise administering an effective amount of compositions described herein, e.g. a composition comprising at least one IL as described herein and an active compound, to a subject in order to alleviate a symptom of a condition described herein. As used herein, “alleviating a symptom” is ameliorating any marker or symptom associated with a condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local or systemic.

In some embodiments of any of the aspects, the administration is transdermal. In some embodiments of any of the aspects, the administration is transdermal, to a mucus membrane (e.g., to a nasal, oral, or vaginal membrane), oral, subcutaneous, intradermal, parenteral, intratumoral, or intravenous.

Oral administration can comprise providing tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Oral formulations can comprise discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of CAGE and the at least one active compound, and may be prepared by methods of pharmacy well known to those skilled in the art. See generally, Remington: The Science and Practice of Pharmacy, 21st Ed., Lippincott, Williams, and Wilkins, Philadelphia PA. (2005).

In one aspect of any of the embodiments, described herein is a method of delivery of at least one active compound by subcutaneous, intradermal or intravenous administration, the method comprising administering the active compound in combination with at least one IL as described herein. In some embodiments of any of the aspects, subcutaneous, intradermal or intravenous administration comprises administration via injection, catheter, port, or the like.

In one aspect of any of the embodiments, described herein is a method of parenteral delivery of at least one active compound, the method comprising parenterally administering the active compound in combination with at least one IL as described herein. In some embodiments, the parenteral administration comprises delivery to a tumor, e.g., a cancer tumor. In some embodiments of any of the aspects, the composition or combination described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition comprising at least one IL (e.g., CAGE) in combination with at least one active compound as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of an ingredient in a composition as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. While as noted above herein, the compositions comprising the at least one IL in combination with at least one active compound can obviate certain reasons for using a controlled-release formulation, it is contemplated herein that the methods and compositions can be utilized in controlled-release formulations in some embodiments. For example, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition comprising the at least one IL in combination with at least one active compound can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to atypical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active compound, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for blood glucose, among others. The dosage can be determined by a physician and adjusted, as necessary, to suit observed effects of the treatment.

As used herein, “diabetes” refers to diabetes mellitus, a metabolic disease characterized by a deficiency or absence of insulin secretion by the pancreas. As used throughout, “diabetes” includes Type 1, Type 2, Type 3, and Type 4 diabetes mellitus unless otherwise specified herein. The onset of diabetes is typically due to a combination of hereditary and environmental causes, resulting in abnormally high blood sugar levels (hyperglycemia). The two most common forms of diabetes are due to either a diminished production of insulin (in type 1), or diminished response by the body to insulin (in type 2 and gestational). Both lead to hyperglycemia, which largely causes the acute signs of diabetes: excessive urine production, resulting compensatory thirst and increased fluid intake, blurred vision, unexplained weight loss, lethargy, and changes in energy metabolism. Diabetes can cause many complications. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation. In some embodiments, the diabetes can be Type 2 diabetes. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. In some embodiments, a subject can be pre-diabetic, which can be characterized, for example, as having elevated fasting blood sugar or elevated post-prandial blood sugar.

Glucagon-Like Peptide-1(GLP-1), is known to reduce food intake and hunger feelings in humans and is an incretin derived from the transcription product of the proglucagon gene that contributes to glucose homeostasis. GLP-1 mimetics are currently being used in the treatment of Type 2 diabetes. Recent clinical trials have shown that these treatments not only improve glucose homeostasis but also succeed in inducing weight loss. As used herein. “GLP-1 polypeptide” refers to the various pre- and pro-peptides and cleavage products of GLP-1, e.g., for human: GLP-1(1-37) (SEQ ID NO: 2), GLP-1 (7-36) (SEQ ID NO: 3), and GLP-1 (7-37) (SEQ ID NO: 4). In some embodiments, a GLP-1 polypeptide can be GLP-1 (7-36) and/or GLP-1 (7-37) or the correlating polypeptides from a species other than human. Sequences for GLP-1 polypeptides are known in the art for a number of species, e.g. human GLP-1 (NCBI Gene ID: 2641) polypeptides (e.g., NCBI Ref Seq: NP_002045.1; SEQ ID NO: 1) and SEQ ID NOs: 2-4. In some embodiments, a pre or pro-peptide of GLP-1 can be used in the methods or compositions described herein, e.g., a glucagon preproprotein (e.g., SEQ ID NO: 1). Naturally-occurring alleles or variants of any of the polypeptides described herein are also specifically contemplated for use in the methods and compositions described herein.

| SEQ ID NO: 1 | |
|---|---|
| 1 | mksiyfvagl fvmlvqgswq rslqdteeks rsfsasqadp lsdpdqmned krhsqgtfts |
| 61 | dyskyldsrr aqdfvqwlmn tkrnrnniak rhdeferhae gtftsdvssy legqaakefi |
| 121 | awlvkgrgrr dfpeevaive elgrrhadgs fsdemntild nlaardfinw liqtkitdrk |
| SEQ ID NO: 2 | |
| | hdeferhae gtftsdvssy legqaakefi awlvkgrg |
| SEQ ID NO: 3 | |
| | hae gtftsdvssy legqaakefi awlvkgr |
| SEQ ID NO: 4 | |
| | hae gtftsdvssy legqaakefi awlvkgrg |

Various GLP-1 mimetics are known in the art and used in the treatment of diabetes. GLP-1 mimetics (or analogues) can include exendin-4 (a Heloderma lizard polypeptide with homology to human GLP-1) and derivatives thereof, GLP-1 analogs modified to be DPP-IV resistant, or human GLP-1 polypeptides conjugated to various further agents, e.g., to extend the half-life. GLP-1 mimetics/analogues can include, e.g., exenatide, lixisenatide, dulaglutide, semaglutide, albiglutide, LY2189265, liraglutide, and taspoglutide. Examples of such molecules and further discussion of their manufacture and activity can be found in the art, e.g., Gupta. Indian J. Endocrinol Metab 17:413-421 (2013); Garber. Diabetes Treatments 41:S279-S284 (2018); US Patent Publication US2009/0181912; and International Patent Publication WO2011/080103, each of which is incorporated by reference herein in its entirety.

In some embodiments of any of the aspects, the active compound can be a chemotherapeutic agent or agent effective for the treatment of cancer. As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm.; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

In some embodiments of any of the aspects, the composition as described herein, e.g., a composition comprising at least one IL as described herein in combination with at least one active compound, is administered as a monotherapy, e.g., another treatment for the condition is not administered to the subject.

In some embodiments of any of the aspects, the methods described herein can further comprise administering a second agent and/or treatment to the subject, e.g. as part of a combinatorial therapy, either in the composition described herein, e.g., a composition comprising at least one IL as described herein in combination with at least one active compound, or as a separate formulation. For example, non-limiting examples of a second agent and/or treatment for treatment of cancer can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition described herein, e.g., a composition comprising at least one IL as described herein in combination with at least one active compound, can be administered to a patient once. In certain embodiments, an effective dose a composition described herein, e.g., a composition comprising at least one IL as described herein in combination with at least one active compound, can be administered to a patient repeatedly. For systemic administration, subjects can be administered a therapeutic amount of a composition described herein, e.g., a composition comprising at least one IL as described herein in combination with at least one active compound, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more. In some embodiments of any of the aspects, the at least one active compound is present in the combination at a dose of from about 1.0-40.0 mg/kg. In some embodiments of any of the aspects, the at least one active compound is present in the combination at a dose of from 1.0-40.0 mg/kg. In some embodiments of any of the aspects, the at least one active compound is present in the combination at a dose of from about 1.0-20.0 mg/kg. In some embodiments of any of the aspects, the at least one active compound is present in the combination at a dose of from 1.0-20.0 mg/kg.

In some embodiments, the active compound is insulin and the concentration or dosage of insulin can be from about 1 U/kg to about 20 U/kg. In some embodiments, the active compound is insulin and the concentration or dosage of insulin can be from 1 U/kg to 20 U/kg. In some embodiments, the active compound is insulin and the concentration or dosage of insulin can be less than 20 U/kg. In some embodiments, the active compound is insulin and the concentration or dosage of insulin can be from about 2 U/kg to about 10 U/kg. In some embodiments, the active compound is insulin and the concentration or dosage of insulin can be from 2 U/kg to 10 U/kg. In some embodiments, the active compound is insulin and the concentration or dosage of insulin can be from about 2 U/kg to about 5 U/kg. In some embodiments, the active compound is insulin and the concentration or dosage of insulin can be from 2 U/kg to 5 U/kg. In some embodiments, the active compound is insulin and the concentration or dosage of insulin can be from about 5 U/kg to about 10 U/kg. In some embodiments, the active compound is insulin and the concentration or dosage of insulin can be from 5 U/kg to 10 U/kg. In some embodiments, the active compound is insulin and the concentration or dosage of insulin can be 2 U/kg, 5 U/kg, or 10 U/kg.

In one aspect of any of the embodiments, described herein is a method of treating a disease in a subject in need thereof by administering to the subject an active compound in combination with the at least one IL as described herein by into the affected tissue by injection. In some embodiments, the affected tissue is tissue comprising diseased cells. In some embodiments, the affected tissue is tissue displaying symptoms of the disease. Non-limiting examples of suitable affected tissues include tumor tissue, fat tissue, adipose tissue, or the like. In some embodiments of any of the aspects, suitable affected tissues include tumor tissue, fat tissue, adipose tissue, or the like. In some embodiments of any of the aspects, the disease is a disease arising from tissue growth, e.g., unwanted, aberrant, or pathological tissue growth. A disease arising from tissue growth can be any disease caused by or characterized by, a rate of tissue growth, location of tissue growth, or pattern/structure of tissue growth which differs from what is normal for that tissue type in a healthy subject. Non-limiting examples of such diseases are tumors, cancer, fat/obesity, and/or hyperplasia. In some embodiments of any of the aspects, such diseases are tumors, cancer, fat/obesity, and/or hyperplasia.

Enzyme inhibitors are a treatment option for a number of conditions, including diabetes, where, for example, insulin-degrading enzyme inhibitors, ACE inhibitors, and alpha-glucosidase inhibitors have all been explored as therapeutic approaches. Safe, effective enzyme inhibitors are therefore of interest in the treatment of a number of conditions. Without wishing to be bound by theory, it is contemplated herein that the ILs described herein can exhibit enzyme inhibition activity. Accordingly, in one aspect of any of the embodiments, described herein is a method of treating diabetes, ulcers, cancer, or fibrosis in a subject in need thereof, the method comprising administering to the subject a composition comprising at least one IL as described herein. In some embodiments, the composition does not comprise a further therapeutically active agent.

Fibrotic conditions benefit from the production and/or maintenance of the extracellular matrix by reducing the accumulation of scar tissue in favor of extracellular matrix. As used herein, “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue. Fibrosis can occur as the result of inflammation, irritation, or healing. A subject in need of treatment for a fibrotic condition is any subject having, or diagnosed as having, or at risk of having a fibrotic condition. Non-limiting examples of fibrotic conditions include, but are not limited to pulmonary fibrosis; scarring; scarring of the skin; trauma; a wound; chronic wounds (e.g. as in diabetes patients), corneal defects; corneal ulceration; corneal wounds; diabetic ulcer; ulcer; sepsis; arthritis; idiopathic pulmonary fibrosis; cystic fibrosis; cirrhosis; endomyocardial fibrosis; mediastinal fibrosis; myelofibrosis; retroperitoneal fibrosis; progressive massive fibrosis; nephrogenic systemic fibrosis; Crohn's disease; keloid; scleroderma; systemic sclerosis; arthrofibrosis; adhesive capsulitis; lung fibrosis; liver fibrosis; kidney fibrosis; heart fibrosis; vascular fibrosis; skin fibrosis; eye fibrosis; bone marrow fibrosis; asthma; sarcoidosis; COPD; emphysema; nschistomasomiasis; cholangitis; diabetic nephropathy; lupus nephritis; postangioplasty aterial restenosis; atherosclerosis; burn scarring; hypertrophic scarring; nephrogenic fibrosing dermatopathy; postcataract surgery; proliferative vitreoretinopathy; Peyronie's disease; Duputren's contracture; dermatomyositis; and graft versus host disease.

As used herein, “ulcer” refers to a break or disruption of a bodily membrane. In some embodiments, the ulcer can be caused by inflammation and/or necrosis of the affected tissue. Ulcers can be skin ulcers (e.g., pressure ulcers, diabetic ulcers, ulcerative dermatitis, and the like), a corneal ulcer, an oral ulcer, a peptic ulcer, a venousucler, a stress ulcer, or ulcerative colitis.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active compound. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition described herein, e.g., a composition comprising at least one IL in combination with at least one active compound, can be administered over a period of time, such as over a 5 minute, 10 minute, 15 minute, 20 minute, or 25 minute period.

The dosage ranges for the administration of the compositions described herein, according to the methods described herein depend upon, for example, the form of the active compound, its potency, and the extent to which symptoms, markers, or indicators of a condition described herein are desired to be reduced, for example the percentage reduction desired for symptoms or markers. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition described in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of diabetes or cancer. When using an experimental animal model, efficacy of treatment is evidenced when a statistically significant change in a marker is observed.

In vitro and animal model assays are provided herein which allow the assessment of a given dose of a composition described herein, e.g., a composition comprising at least one IL in combination with at least one active compound.

In some embodiments of any of the aspects, the subject administered a composition comprising at least one IL as described herein, e.g., in combination with an active compound is a subject having, diagnosed as having, or in need of treatment for obesity, excess weight, or prevention of weight gain. In some embodiments, the subject is overweight. The methods described herein comprises methods of treating obesity, reducing weight gain, preventing weight gain, promoting weight loss, and the like. Such methods can, e.g., promote metabolic health, be pursued for aesthetic reasons, and/or prepare patients for surgical interventions which are counter indicated for those with high BMIs or weights. In some embodiments, weight loss can be medically necessary and/or medically indicated, e.g. when the subject is overweight and/or obese. In some embodiments, weight loss can be for cosmetic purposes, e.g. when the subject desires to lose weight whether or not weight loss is medically necessary and/or medically indicated.

The term “obesity” refers to excess fat in the body. Obesity can be determined by any measure accepted and utilized by those of skill in the art. Currently, an accepted measure of obesity is body mass index (BMI), which is a measure of body weight in kilograms relative to the square of height in meters. Generally, for an adult over age 20, a BMI between about 18.5 and 24.9 is considered normal, a BMI between about 25.0 and 29.9 is considered overweight, a BMI at or above about 30.0 is considered obese, and a BMI at or above about 40 is considered morbidly obese. (See, e.g., Gallagher et al. (2000) Am J Clin Nutr 72:694-701.) These BMI ranges are based on the effect of body weight on increased risk for disease. Some common conditions related to high BMI and obesity include cardiovascular disease, high blood pressure (i.e., hypertension), osteoarthritis, cancer, and diabetes. Although BMI correlates with body fat, the relation between BMI and actual body fat differs with age and gender. For example, women are more likely to have a higher percent of body fat than men for the same BMI. Furthermore, the BMI threshold that separates normal, overweight, and obese can vary, e.g. with age, gender, ethnicity, fitness, and body type, amongst other factors. In some embodiments, a subject with obesity can be a subject with a body mass index of at least about 25 kg/m² prior to administration of a treatment as described herein. In some embodiments, a subject with obesity can be a subject with a body mass index of at least about 30 kg/m² prior to administration of a treatment as described herein.

In some embodiments of any of the aspects, the subject administered a composition comprising at least one IL as described herein, e.g., in combination with at least one active compound is a subject having, diagnosed as having, or in need of treatment for a metabolic disorder or metabolic syndrome. The term “metabolic disorder” refers to any disorder associated with or aggravated by impaired or altered glucose regulation or glycemic control, such as, for example, insulin resistance. Such disorders include, but are not limited to obesity; excess adipose tissue; diabetes; fatty liver disease; non-alcoholic fatty liver disease; metabolic syndrome; dyslipidemia; hypertension; hyperglycemia; and cardiovascular disease. “Metabolic syndrome”, which is distinct from metabolic disorder, refers to a combination of medical disorders that, when occurring together, increase the risk of developing cardiovascular disease and diabetes. A number of definitions of metabolic syndrome have been established, e.g., by the American Heart Association and the International Diabetes Foundation. As but one example, the WHO defines metabolic syndrome as the presence of any one of diabetes mellitus, impaired glucose tolerance, impaired fasting glucose or insulin resistance and two of the following: blood pressure equal to or greater than 140/90 mmHg, dyslipidemia, central obesity, and microalbuminuria. In some embodiments, the metabolic disorder can be selected from the group consisting of: obesity; excess adipose tissue; diabetes; and cardiovascular disease.

The uptake of many active compounds, e.g., pharmaceutically active compounds, can be improved by delivering the compounds in solvents. However, such approaches are often unsuitable for in vivo use because most such solvents demonstrate toxic side effects and/or act as irritants to the point of delivery. Described herein are methods and compositions which can provide low toxicity with improved delivery kinetics.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

A carboxylic acid is a carbonyl-bearing functional group having a formula RCOOH where R is aliphatic, heteroaliphatic, alkyl, or heteroalkyl.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF3, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y} alkenyl are typically used where X and Y indicate the number of carbon atoms in the chain. For example, C₂-C₆alkenyl includes alkenyls that have a chain of between 1 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 1 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine. A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cylcyl are typically used where X and Y indicate the number of carbon atoms in the ring system. The cycloalkyl group additionally can be optionally substituted, e.g., with 1, 2, 3, or 4 substituents. Examples of cyclyl groups include, without limitation, cyclopropyl, cyclobutyl, cyclopentyl, cyclopentenyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies. As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. Heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo [2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

As used herein, the term “substituted” refers to independent replacement of one or more of the hydrogen atoms on the substituted moiety with substituents independently selected from, but not limited to, alkyl, alkenyl, heterocycloalkyl, alkoxy, aryloxy, hydroxy, amino, amido, alkylamino, arylamino, cyano, halo, mercapto, nitro, carbonyl, acyl, aryl and heteroaryl groups.

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls (including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O— alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like. The term “alkoxycarbonyl” means —C(O)O-(alkyl), such as —C(═O)OCH₃, —C(═O)OCH₂CH₃, and the like. The term “alkoxyalkyl” means -(alkyl)-O-(alkyl), such as —CH₂OCH₃, —CH₂OCH₂CH₃, and the like. The term “aryloxy” means —O-(aryl), such as —O-phenyl, —O-pyridinyl, and the like. The term “arylalkyl” means -(alkyl)-(aryl), such as benzyl (i.e., —CH₂phenyl), —CH₂-pyrindinyl, and the like. The term “arylalkyloxy” means —O-(alkyl)-(aryl), such as —O-benzyl, —O—CH₂-pyridinyl, and the like. The term “cycloalkyloxy” means —O-(cycloalkyl), such as —O-cyclohexyl, and the like. The term “cycloalkylalkyloxy” means —O-(alkyl)-(cycloalkyl, such as —OCH2cyclohexyl, and the like. The term “aminoalkoxy” means —O-(alkyl)-NH₂, such as —OCH₂NH₂, —OCH₂CH₂NH₂, and the like. The term “mono- or di-alkylamino” means —NH(alkyl) or —N(alkyl)(alkyl), respectively, such as —NHCH₃, —N(CH₃)₂, and the like. The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like. The term “arylamino” means —NH(aryl), such as —NH-phenyl, —NH-pyridinyl, and the like. The term “arylalkylamino” means —NH-(alkyl)-(aryl), such as —NH-benzyl, —NHCH₂-pyridinyl, and the like. The term “alkylamino” means —NH(alkyl), such as —NHCH₃, —NHCH₂CH₃, and the like. The term “cycloalkylamino” means —NH-(cycloalkyl), such as —NH-cyclohexyl, and the like. The term “cycloalkylalkylamino”-NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH3) as well as —CRₐR_{b}Rₑ where Rₐ, R_{b}, and Rₑ can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane polarized light in different directions). Enantiomers generally have identical physical properties, such as melting points and boiling points, and also have identical spectroscopic properties. Enantiomers can differ from each other with respect to their interaction with plane-polarized light and with respect to biological activity.

The term “racemic mixture”, “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “resolving” or “resolution” when used in reference to a racemic mixture refers to the separation of a racemate into its two enantiomorphic forms (i.e., (+) and (−); or (R) and (S) forms). The terms can also refer to enantioselective conversion of one isomer of a racemate to a product.

The term “enantiomeric excess” or “ee” refers to a reaction product wherein one enantiomer is produced in excess of the other, and is defined for a mixture of (+)- and (−)-enantiomers, with composition given as the mole or weight or volume fraction F₍₊₎ and F₍₋₎ (where the sum of F₍₊₎ and F₍₋₎=1). The enantiomeric excess is defined as *F₍₊₎ —F₍₋₎* and the percent enantiomeric excess by 100x*F₍₊₎ —F₍₋₎*. The “purity” of an enantiomer is described by its ee or percent ee value (% ee).

Whether expressed as a “purified enantiomer” or a “pure enantiomer” or a “resolved enantiomer” or “a compound in enantiomeric excess”, the terms are meant to indicate that the amount of one enantiomer exceeds the amount of the other. Thus, when referring to an enantiomer preparation, both (or either) of the percent of the major enantiomer (e.g. by mole or by weight or by volume) and (or) the percent enantiomeric excess of the major enantiomer may be used to determine whether the preparation represents a purified enantiomer preparation.

The term “enantiomeric purity” or “enantiomer purity” of an isomer refers to a qualitative or quantitative measure of the purified enantiomer; typically, the measurement is expressed on the basis of ee or enantiomeric excess.

The terms “substantially purified enantiomer”, “substantially resolved enantiomer” “substantially purified enantiomer preparation” are meant to indicate a preparation (e.g. derived from non-optically active starting material, substrate, or intermediate) wherein one enantiomer has been enriched over the other, and more preferably, wherein the other enantiomer represents less than 20%, more preferably less than 10%, and more preferably less than 5%, and still more preferably, less than 2% of the enantiomer or enantiomer preparation.

The terms “purified enantiomer”, “resolved enantiomer” and “purified enantiomer preparation” are meant to indicate a preparation (e.g. derived from non-optically active starting material, substrates or intermediates) wherein one enantiomer (for example, the R-enantiomer) is enriched over the other, and more preferably, wherein the other enantiomer (for example the S-enantiomer) represents less than 30%, preferably less than 20%, more preferably less than 10% (e.g. in this particular instance, the R-enantiomer is substantially free of the S-enantiomer), and more preferably less than 5% and still more preferably, less than 2% of the preparation. A purified enantiomer may be synthesized substantially free of the other enantiomer, or a purified enantiomer may be synthesized in a stereopreferred procedure, followed by separation steps, or a purified enantiomer may be derived from a racemic mixture.

The term “enantioselectivity”, also called the enantiomeric ratio indicated by the symbol “E”, refers to the selective capacity of an enzyme to generate from a racemic substrate one enantiomer relative to the other in a product racemic mixture; in other words, it is a measure of the ability of the enzyme to distinguish between enantiomers. A nonselective reaction has an E of 1, while resolutions with E's above 20 are generally considered useful for synthesis or resolution. The enantioselectivity resides in a difference in conversion rates between the enantiomers in question. Reaction products are obtained that are enriched in one of the enantiomers; conversely, remaining substrates are enriched in the other enantiomer. For practical purposes it is generally desirable for one of the enantiomers to be obtained in large excess. This is achieved by terminating the conversion process at a certain degree of conversion.

CAGE (Choline And GEranate) is an ionic liquid comprising the cation choline (see, e.g., Formula XI) and the anion geranate or geranic acid (see, e.g., Formulas XII and XIII). Preparation of CAGE can be, e.g., as described in International Patent Publication WO 2015/066647; which is incorporated by reference herein in its entirety, or as described in the examples herein.

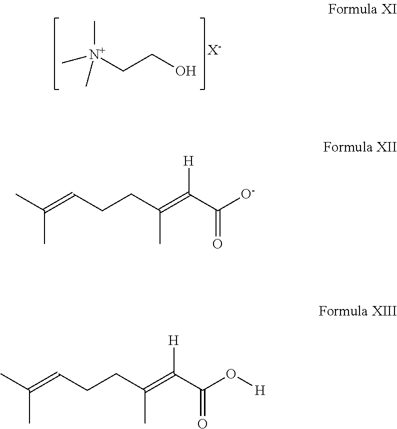

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

In some embodiments of any of the aspects, a variant can be a polypeptide having at least 90%, at least 95%, at least 98% or greater sequence homology to one of the reference sequences provided herein and retaining the wild-type activity of that reference sequence, e.g., incretin activity. In some embodiments of any of the aspects, a variant can be a polypeptide having at least 90%, at least 95%, at least 98% or greater sequence homology to one of the naturally-occurring reference sequences provided herein and retaining the wild-type activity of that reference sequence, e.g., incretin activity. In some embodiments of any of the aspects, a variant can be a naturally-occurring polypeptide having at least 90%, at least 95%, at least 98% or greater sequence homology to one of the reference sequences provided herein and retaining the wild-type activity of that reference sequence, e.g., incretin activity.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof, including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies. Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

Antibodies and/or antibody reagents can include an immunoglobulin molecule, a monoclonal antibody, a chimeric antibody, a CDR-grafted antibody, a humanized antibody, a fully human antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

As used herein, the term “nanobody” or single domain antibody (sdAb) refers to an antibody comprising the small single variable domain (VHH) of antibodies obtained from camelids and dromedaries. Antibody proteins obtained from members of the camel and dromedary (Camelus baclrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994; which is incorporated by reference herein in its entirety).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J. 17: 3512-3520; each of which is incorporated by reference herein in its entirety. Engineered libraries of camelid antibodies and antibody fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”. Thus the natural low antigenicity of camelid antibodies to humans can be further reduced.

The camelid nanobody has a molecular weight approximately one-tenth that of a human IgG molecule and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody. The low molecular weight and compact size further result in camelid nanobodies being extremely thermostable, stable to extreme pH and to proteolytic digestion, and poorly antigenic. See U.S. patent application 20040161738 published Aug. 19, 2004; which is incorporated by reference herein in its entirety. These features combined with the low antigenicity to humans indicate great therapeutic potential.

In some embodiments of any of the aspects, the active compound comprises an antibody or antibody reagent and the anion is a hydrophobic anion comprising carboxylic acid having a pKa of at least 4.0 and a Log P of at least 1.0. In some embodiments of any of the aspects, the active compound comprises an antibody or antibody reagent and the anion is hexenoic acid.

In some embodiments of any of the aspects, the active compound comprises a nucleic acid and the anion is a hydrophobic anion comprising carboxylic acid having a pKa of at least 4.0 and a Log P of at least 1.0. In some embodiments of any of the aspects, the active compound comprises a nucleic acid and the anion is hexenoic acid.

In some embodiments of any of the aspects, the active compound comprises an inhibitory nucleic acid, siRNA, pDNA, or mRNA and the anion is a hydrophobic anion comprising carboxylic acid having a pKa of at least 4.0 and a Log P of at least 1.0. In some embodiments of any of the aspects, the active compound comprises an inhibitory nucleic acid, siRNA, pDNA, or mRNA and the anion is hexenoic acid.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., cDNA. Suitable RNA can include, e.g., mRNA.

As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art. One skilled in the art would be able to design further siRNA, shRNA, or miRNA to target the nucleic acid sequence of a target gene or gene product (e.g., mRNA), e.g., using publically available design tools. siRNA, shRNA, or miRNA is commonly made using companies such as Dharmacon (Layfayette, CO) or Sigma Aldrich (St. Louis, MO).

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2- [known as a methylene (methylimino) or MMI backbone], —CH2-O—N(CH3)-CH2-, —CH2-N(CH3)-N(CH3)-CH2- and —N(CH3)-CH2-CH2- [wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mol Cane Ther 6(3):833-843; Grunweller, A. et al., (2003) Nucleic Acids Research 31(12):3185-3193).

Modified RNAs can also contain one or more substituted sugar moieties. The iRNAs, e.g., dsRNAs, described herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Exemplary suitable modifications include O[(CH2)nO] mCH3, O(CH2)·nOCH3, O(CH2)nNH2, O(CH2) nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2)2ON(CH3)2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2)2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), athioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target.

Any CRISPR-associated nuclease can be used in the system and methods of the invention. CRISPR nuclease systems are known to those of skill in the art, e.g. Cas9, Cas12, Cas12a, or the like, see Patents/applications 8,993,233, US 2015/0291965, US 2016/0175462, US 2015/0020223, US 2014/0179770, 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off_Spotter™; and Synthego™; which are available on the world wide web).

The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a recombinant vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a condition or disease described herein. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “effective amount” means an amount of a composition sufficient to provide at least some amelioration of the symptoms associated with the condition. In one embodiment, the “effective amount” means an amount of a composition would decrease the markers or symptoms of the condition in a subject having the condition.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” or “comprises” is used in reference to methods and compositions, and respective component(s) thereof, that are essential to the invention, yet open to the inclusion of unspecified elements, whether essential or not. As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4* ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A composition comprising at least one ionic liquid comprising:
   an anion which is at least one of:
      a) a carboxylic acid which is not a fatty acid; and
      b) a hydrophobic anion comprising carboxylic acid having a pKa of at least 4.0 and a Log P of at least 1.0;
   a cation which is acetylcholine.
2. The composition of paragraph 1, wherein the anion is a carboxylic acid which is not a fatty acid.
3. The composition of paragraph 2, wherein the anion has a Log P of less than 1.0.
The composition of any one of paragraph 2-3, wherein the fatty acid comprises an aliphatic chain of no more than 3 carbons.
4. The composition of anyone of paragraphs 2-3, wherein the anion comprises only one carboxylic acid group (e.g., R—COOH group).
5. The composition of any one of paragraphs 2-4, wherein the anion is selected from the group consisting of:
   lactic acid; glycolic acid; malonic acid; maleic acid; glutaric acid; citric acid;
   gluconic acid; and adipic acid.
6. The composition of any one of paragraphs 2-5, wherein the anion is maleic acid.
7. The composition of paragraph 1, wherein the anion is a hydrophobic anion comprising carboxylic acid having a pKa of at least 4.0 and a Log P of at least 1.0 and has a pKa of at least 4.5.
8. The composition of paragraph 7, wherein the anion has a pKa of at least 5.0.
9. The composition of any one of paragraphs 7-8, wherein the anion comprises a carbon chain of at least 8 carbons.
10. The composition of any one of paragraphs 7-9, wherein the anion comprises a carbon chain with an 8 carbon backbone.
11. The composition of any one of paragraphs 7-10, wherein the anion is geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, or hexenoic acid.
12. The composition of any one of paragraphs 7-10, wherein the anion is hexenoic acid.
13. The composition of any of the preceding paragraphs, wherein the ionic liquid comprises a ratio of cation to anion of from about 2:1 to about 1:1.
14. The composition of any of the preceding paragraphs, wherein the ionic liquid comprises a ratio of cation to anion of about 2:1.
15. The composition of any of the preceding paragraphs, wherein the ionic liquid has a cation:anion ratio of less than 1:1.
16. The composition of any of the preceding paragraphs, wherein the ionic liquid has a cation:anion ratio with an excess of cation.
17. The composition of any of the preceding paragraphs, comprising a first ionic liquid and at least a second ionic liquid.
18. The composition of paragraph 17, wherein the first ionic liquid and the second ionic liquid each comprise a different anion.
19. The composition of any of the preceding paragraphs, further comprising at least one active compound in combination with the at least one ionic liquid.
20. The composition of paragraph 19, wherein the active compound comprises a polypeptide.
21. The composition of paragraph 20, wherein the polypeptide is an antibody or antibody reagent.
22. The composition of any one of paragraphs 19-21, wherein the active compound has a molecular weight of greater than 450.
23. The composition of any one of paragraphs 19-22, wherein the active compound has a molecular weight of greater than 500.
24. The composition of any one of paragraphs 19-23, wherein the active compound comprises insulin, acarbose, ruxolitinib, or a GLP-1 polypeptide or mimetic or analog thereof.
25. The composition of any one of paragraphs 19-23, wherein the anion is a hydrophobic anion comprising carboxylic acid having a pKa of at least 4.0 and a Log P of at least 1.0, and the active compound comprises an antibody or antibody reagent.
26. The composition of paragraph 19, wherein the active compound comprises a nucleic acid.
27. The composition of paragraph 26, wherein the nucleic acid is an inhibitory nucleic acid.
28. The composition of paragraph 27, wherein the nucleic acid is a siRNA, pDNA, or mRNA.
29. The composition of any one of paragraphs 26-28, wherein the anion is a hydrophobic anion comprising carboxylic acid having a pKa of at least 4.0 and a Log P of at least 1.0 and the active compound comprises a nucleic acid.
30. The composition of any of the preceding paragraphs, wherein the ionic liquid is at a concentration of at least 0.1% w/v.
31. The composition of any of the preceding paragraphs, wherein the ionic liquid is at a concentration of from about 10 to about 70% w/v.
32. The composition of any of the preceding paragraphs, wherein the ionic liquid is at a concentration of from about 30 to about 50% w/v.
33. The composition of any of the preceding paragraphs, wherein the ionic liquid is at a concentration of from about 30 to about 40% w/v.
34. The composition of any of the preceding paragraphs, wherein the composition is formulated for administration transdermally, to a mucus membrane, orally, subcutaneously, intradermally, parenterally, intratumorally, or intravenously.
35. The composition of paragraph 34, wherein the composition is formulated for transdermal administration.
36. The composition of paragraph 34, wherein the mucus membrane is nasal, oral, or vaginal.
37. The composition of any of the preceding paragraphs, wherein the active compound is provided at a dosage of 1-40 mg/kg.
38. The composition of any of the preceding paragraphs, further comprising at least one non-ionic surfactant.
39. The composition of any of the preceding paragraphs, further comprising a pharmaceutically acceptable carrier.
40. The composition of any of the preceding paragraphs, wherein the composition is provided in a degradable capsule.
41. The composition of any of the preceding paragraphs, wherein the composition is an admixture.
42. The composition of any of the preceding paragraphs, wherein the composition is provided in one or more nanoparticles.
43. The composition of any of the preceding paragraphs, comprising one or more nanoparticles comprising the active compound, the nanoparticles in solution or suspension in a composition comprising the ionic liquid.
44. A method of administering at least one active compound to a subject, the method comprising administering a composition of any of paragraphs 1-43.
45. The method of paragraph 44, wherein the composition is administered once.
46. The method of any of paragraphs 44-45, wherein the composition is administered in multiple doses.
47. The method of any of paragraphs 44-46, wherein the administering is transdermally, to a mucus membrane, orally, subcutaneously, intradermally, parenterally, intratumorally, or intravenously.
48. A composition of any of paragraphs 1-43 for use in a method of administering at least one active compound to a subject.
49. The composition of paragraph 48, wherein the composition is administered once.
50. The composition of any of paragraphs 48-49, wherein the composition is administered in multiple doses.
51. The composition of any of paragraphs 48-50, wherein the administering is transdermally, to a mucus membrane, orally, subcutaneously, intradermally, parenterally, intratumorally, or intravenously.

In some embodiments, the present technology may be defined in any of the following numbered paragraphs:
1. A composition comprising at least one ionic liquid comprising:
   an anion which is at least one of:
      a) a carboxylic acid which is not a fatty acid; and
      b) a hydrophobic anion comprising carboxylic acid having a pKa of at least 4.0 and a Log P of at least 1.0;
   a cation which is a quaternary ammonium comprising an ester group.
2. The composition of paragraph 1, wherein the cation is acetylcholine.
3. The composition of any one of paragraphs 1-2, wherein the anion is a carboxylic acid which is not a fatty acid.
4. The composition of paragraph 3, wherein the anion has a Log P of less than 1.0.
5. The composition of any one of paragraph 3-4, wherein the anion comprises an aliphatic chain of no more than 3 carbons.
6. The composition of any one of paragraphs 3-5, wherein the anion comprises only one carboxylic acid group (e.g., R—COOH group).
7. The composition of any one of paragraphs 3-6, wherein the anion is selected from the group consisting of:
   lactic acid; glycolic acid; malonic acid; maleic acid; glutaric acid; citric acid;
   gluconic acid; propanoic acid; and adipic acid.
8. The composition of any one of paragraphs 3-7, wherein the anion is maleic acid.
9. The composition of any one of paragraphs 3-8, wherein the anion is propanoic acid.
10. The composition of any one of paragraphs 1-2, wherein the anion is a hydrophobic anion comprising carboxylic acid having a pKa of at least 4.0 and a Log P of at least 1.0 and has a pKa of at least 4.5.
11. The composition of paragraph 10, wherein the anion has a pKa of at least 5.0.
12. The composition of any one of paragraphs 10-11, wherein the anion comprises a carbon chain of at least 8 carbons.
13. The composition of any one of paragraphs 10-12, wherein the anion comprises a carbon chain with an 8 carbon backbone.
14. The composition of any one of paragraphs 10-13, wherein the anion is geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, or hexenoic acid.
15. The composition of any one of paragraphs 10-14, wherein the anion is hexenoic acid.
16. The composition of any one of the preceding paragraphs, wherein the ionic liquid comprises a ratio of cation to anion of from about 2:1 to about 1:1.
17. The composition of any one of the preceding paragraphs, wherein the ionic liquid comprises a ratio of cation to anion of about 2:1.
18. The composition of any one of the preceding paragraphs, wherein the ionic liquid has a cation:anion ratio of less than 1:1.
19. The composition of any one of the preceding paragraphs, wherein the ionic liquid has a cation:anion ratio with an excess of cation.
20. The composition of any one of the preceding paragraphs, comprising a first ionic liquid and at least a second ionic liquid.
21. The composition of paragraph 20, wherein the first ionic liquid and the second ionic liquid each comprise a different anion.
22. The composition of any one of the preceding paragraphs, further comprising at least one active compound in combination with the at least one ionic liquid.
23. The composition of paragraph 22, wherein the active compound comprises a polypeptide.
24. The composition of paragraph 23, wherein the polypeptide is an antibody or antibody reagent.
25. The composition of any one of paragraphs 22-24, wherein the active compound has a molecular weight of greater than 450.
26. The composition of any one of paragraphs 22-25, wherein the active compound has a molecular weight of greater than 500.
27. The composition of any one of paragraphs 22-26, wherein the active compound comprises insulin, acarbose, ruxolitinib, or a GLP-1 polypeptide or mimetic or analog thereof.
28. The composition of any one of paragraphs 22-27, wherein the anion is a hydrophobic anion comprising carboxylic acid having a pKa of at least 4.0 and a Log P of at least 1.0, and the active compound comprises an antibody or antibody reagent.
29. The composition of any one of paragraphs 22-28, wherein the anion is hexenoic acid, and the active compound comprises an antibody or antibody reagent.
30. The composition of any one of paragraphs 22-29, wherein the anion is a hydrophobic anion comprising carboxylic acid having a pKa of at least 4.0 and a Log P of at least 1.0, the ionic liquid is present at a concentration of less than 10% w/v, and the active compound comprises an antibody or antibody reagent.
31. The composition of any one of paragraphs 22-30, wherein the anion is hexenoic acid, the ionic liquid is present at a concentration of less than 10% w/v, and the active compound comprises an antibody or antibody reagent.
32. The composition of paragraph 22, wherein the active compound comprises a nucleic acid.
33. The composition of paragraph 32, wherein the nucleic acid is an inhibitory nucleic acid.
34. The composition of paragraph 32 or 33, wherein the nucleic acid is a siRNA, pDNA, or mRNA.
35. The composition of any one of paragraphs 32-34, wherein the anion is a hydrophobic anion comprising carboxylic acid having a pKa of at least 4.0 and a Log P of at least 1.0 and the active compound comprises a nucleic acid.
36. The composition of any one of the preceding paragraphs, wherein the ionic liquid is at a concentration of at least 0.1% w/v.
37. The composition of any one of the preceding paragraphs, wherein the ionic liquid is at a concentration of from about 10 to about 70% w/v.
38. The composition of any one of the preceding paragraphs, wherein the ionic liquid is at a concentration of from about 30 to about 50% w/v.
39. The composition of any one of the preceding paragraphs, wherein the ionic liquid is at a concentration of from about 30 to about 40% w/v.
40. The composition of any one of the preceding paragraphs, wherein the ionic liquid is at a concentration of less than 10% w/v.
41. The composition of any one of the preceding paragraphs, wherein the composition is formulated for administration transdermally, to a mucus membrane, orally, subcutaneously, intradermally, parenterally, intratumorally, or intravenously.
42. The composition of paragraph 41, wherein the composition is formulated for subcutaneous administration.
43. The composition of paragraph 41, wherein the composition is formulated for transdermal administration.
44. The composition of paragraph 41, wherein the mucus membrane is nasal, oral, or vaginal.
45. The composition of any one of the preceding paragraphs, wherein the active compound is provided at a dosage of 1-40 mg/kg.
46. The composition of any one of the preceding paragraphs, further comprising at least one non-ionic surfactant.
47. The composition of any one of the preceding paragraphs, further comprising a pharmaceutically acceptable carrier.
48. The composition of any one of the preceding paragraphs, wherein the composition is provided in a degradable capsule.
49. The composition of any one of the preceding paragraphs, wherein the composition is an admixture.
50. The composition of any one of the preceding paragraphs, wherein the composition is provided in one or more nanoparticles.
51. The composition of any one of the preceding paragraphs, comprising one or more nanoparticles comprising the active compound, the nanoparticles in solution or suspension in a composition comprising the ionic liquid.
52. A method of administering at least one active compound to a subject, the method comprising administering a composition of any one of paragraphs 1-51.
53. The method of paragraph 52, wherein the composition is administered once.
54. The method of any one of paragraphs 52-53, wherein the composition is administered in multiple doses.
55. The method of any one of paragraphs 52-54, wherein the administering is transdermally, to a mucus membrane, orally, subcutaneously, intradermally, parenterally, intratumorally, or intravenously.
56. The method of any one of paragraphs 52-55, wherein the administering is subcutaneous.
57. A composition of any one of paragraphs 1-51 for use in a method of administering at least one active compound to a subject.
58. The composition of paragraph 57, wherein the composition is administered once.
59. The composition of any one of paragraphs 57-58, wherein the composition is administered in multiple doses.
60. The composition of any one of paragraphs 57-59, wherein the administering is transdermally, to a mucus membrane, orally, subcutaneously, intradermally, parenterally, intratumorally, or intravenously.
61. The composition of any one of paragraphs 57-60, wherein the administering is subcutaneous.

The technology described herein is further illustrated by the following examples which in no way should be construed as being further limiting.

### EXAMPLES

### Example 1

Ionic liquids are referred to herein with a notation as follows: x% CA y:z, wherein x is the volume percent of the ionic liquid in formulation (e.g., volume percent in water), C is the cation, A is the anion, and the ratio of y:z is the ratio of cation to anion.

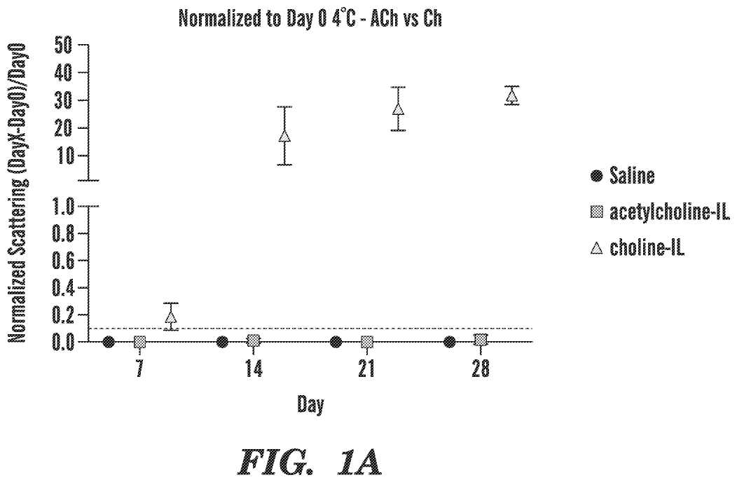

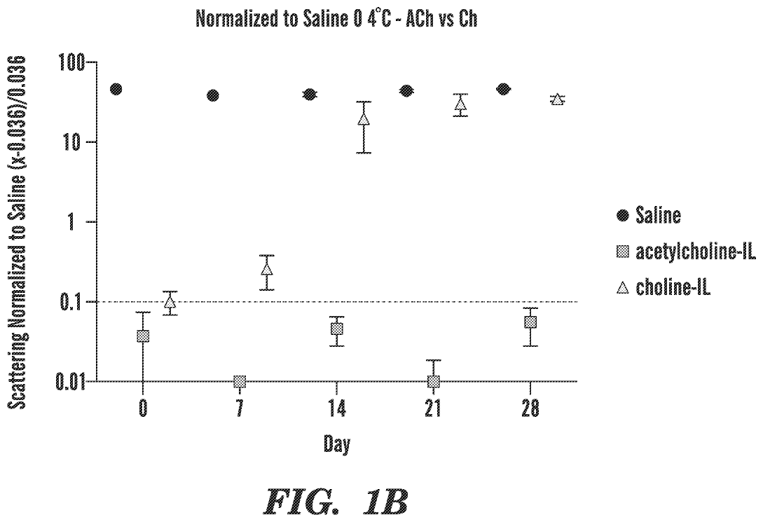

Insulin was formulated in choline glycolic acid and acetylcholine glycolic acid at 100 U/mL (3.4 mg/mL) and stability was examined (FIG. 1). Long term assessment of formulations was performed by quantifying the light scattered. By measuring the absorbance at 540 nm it was assessed whether the insulin monomers are aggregating over time. The data was analyzed in two ways to assess how the scattering changed over time (top panel) and how the formulation clarity changed compared to saline (bottom panel and the ideal formulation clarity). An ideal formulation would see little to no change in normalized scattering and less than 0.1, equal to 10%, change in scattering compared to saline without insulin (absorbance of 0.036). The data shows that the acetylcholine versions of the deep eutectic solvents provide better insulin stability over the longer term (28 days) at relevant storage conditions.

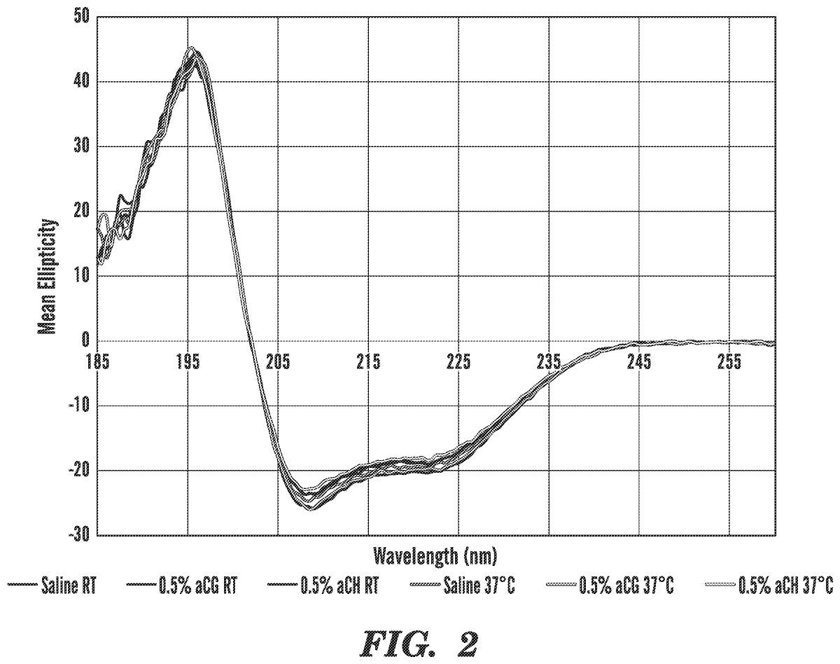

Circular dichroism was used to assess insulin secondary structure (FIG. 2). The experiment was performed after incubation at room temperature and at 37° C. (physiological temperature) for one hour. This was done to assess whether the heat change associated with subcutaneous injection may affect the insulin structure. If there is no significant change in secondary structure than the curves of the experimental deep eutectic solvent groups should match the saline negative control groups, which they do. “aCG” refers to acetylcholine glycolic acid and “aCH” refers to acetylcholine hexenoic acid. Insulin concentration was ˜6 U/mL (0.2 mg/mL).

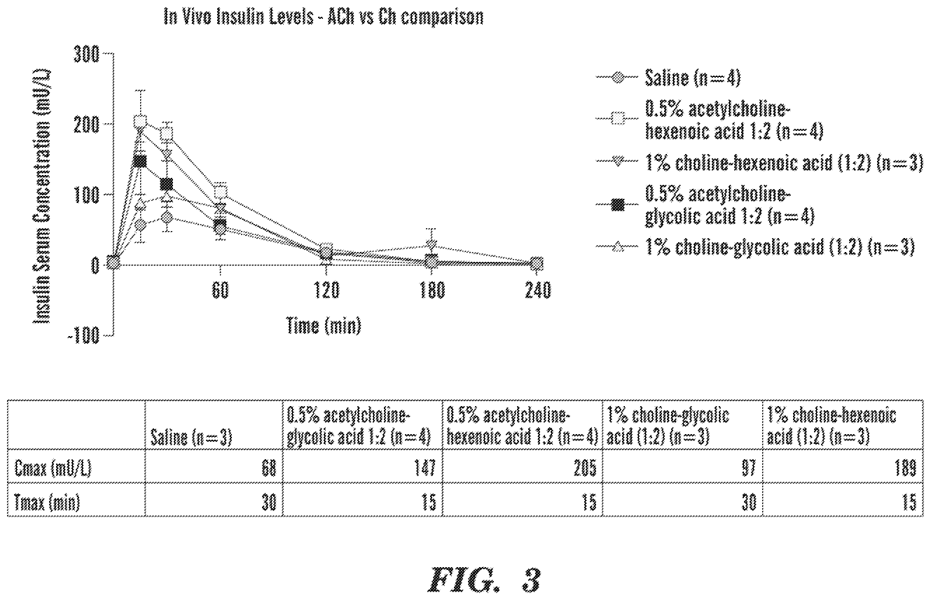

The in vivo PK data indicates that acetylcholine deep eutectic solvents provide a faster insulin delivery method than ILs with a choline cation (FIG. 3). Rats received subcutaneous injections and serum insulin levels were measured using ELISA at time points of 0, 15, 30, 60, 120, 180, and 240 minutes. The average maximum serum concentration (Cmax) and time of average maximum serum concentration (Tmax) are noted in the data table in FIG. 3. The acetylcholine hexenoic acid group had a 20% greater Cmax than the choline variant. And the acetylcholine glycolic acid group had a 50% increase in Cmax and a Tmax that was 15 minutes earlier than the choline variant. Both these data indicate that acetylcholine help to increase serum insulin absorption soon after subcutaneous injection. Insulin concentration was 1 U/kg in non-fasted rats.

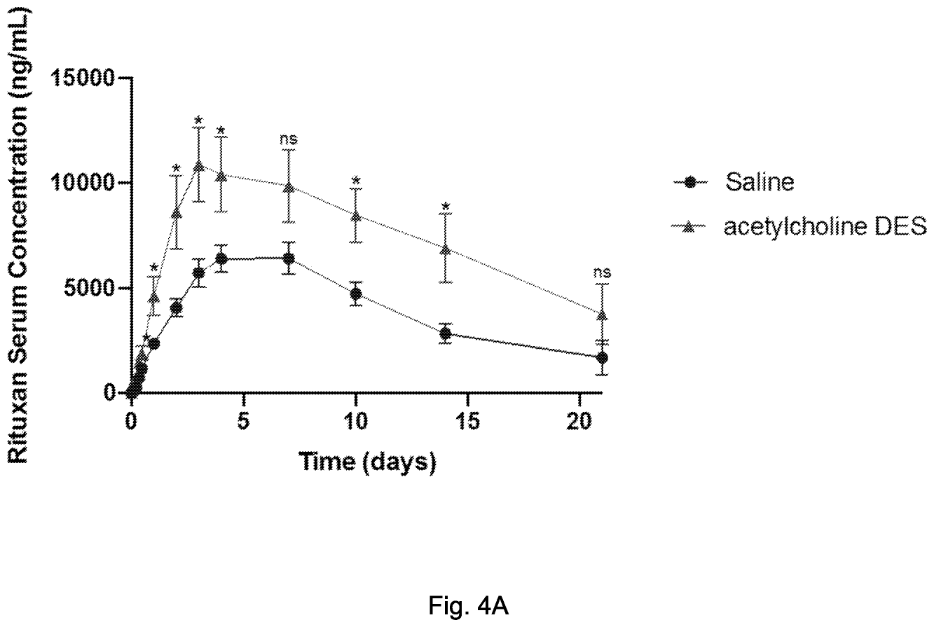

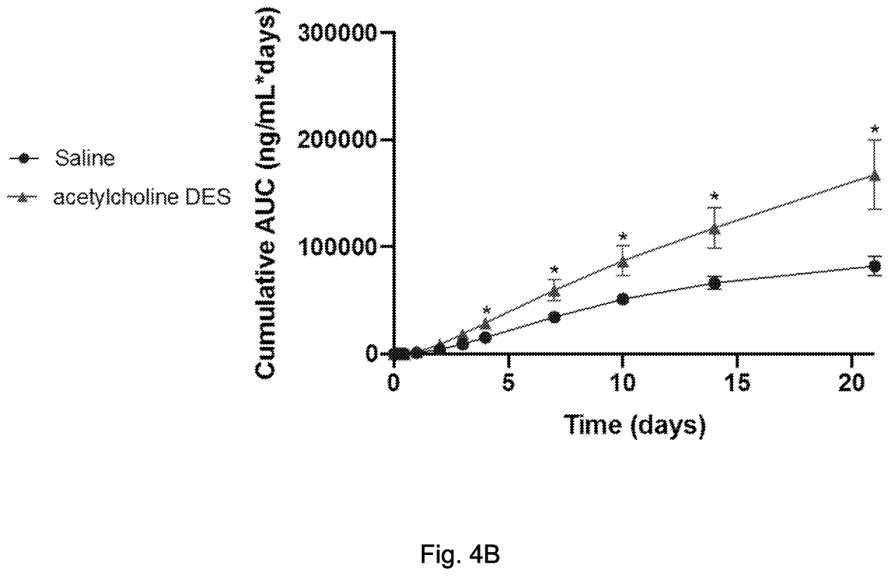

Antibody delivery was tested next. Antibodies at a dose of 10 U/kg were administered to Wister rats, in saline solution or acetylcholine hexenoic acid. The in vivo PK data indicates that acetylcholine deep eutectic solvents allow for increased bioavailability of monoclonal antibodies (FIG. 4). Rats received subcutaneous injections and serum Rituxumab (anti-CD20 antibody) levels were measured using ELISA at time points of 0, 1, 2, 5, 8, 11 hours and 1, 2, 3, 4, 7, 10, 14, 21 days. As seen in the left panel of FIG. 4, the peak of the average serum concentrations for the saline control and the acetylcholine DES formulation are 6,415 and 10,893 ng/mL, respectively. This increased peak concentration leads allows for greater accumulation on the monoclonal antibody in the blood, as indicated by higher AUC at day 4 and beyond. The AUC at day 21 was 103% higher in the acetylcholine DES group than the saline control.

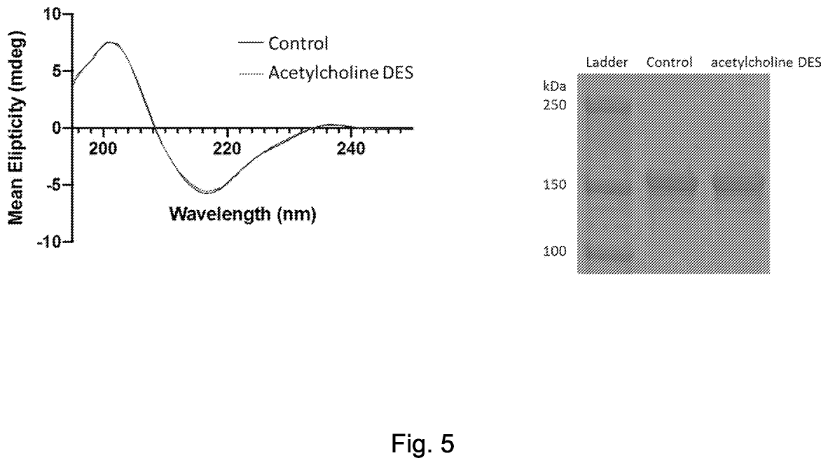

Antibody stability was next tested. Circular dichroism and SDS-PAGE were used to assess antibody stability (FIG. 5). Circular dichroism (FIG. 5, left) indicates that the secondary structure of the antibody is not affected by the acteylcholine hexenoic acid DES formulation compared to “fresh” antibody control. Additionally, SDS-PAGE was used to assess whether acetylcholine caused aggregation (of particular importance for large beta-sheet biologics like antibodies). Again the acetylcholine DES formulation looked the same as the control. Both CD and SDS-PAGE were performed after incubation at room temperature for one hour.

IL delivery of mRNA in vitro was assessed. The in vitro transfection experiments were performed in a dendritic cell line with mRNA nanoparticles and indicate that acetylcholine hexenoic acid DES can enhance mRNA treatments. The particles were incubated with acetylcholine DES solutions to “coat” the particles. The cells were incubated with the particles for the 4 hours before analyzing with flow cytometry. The increased percent of cells expressing GFP indicates that the acetylcholine DES increased delivery into the cells. The higher mean fluorescent intensity shows that the acetylcholine DES helped increase expression of the GFP.

### Example 2

Proteins are among the most common therapeutics for the treatment of diabetes, autoimmune diseases, cancer, and metabolic diseases, among others. Despite their common use, current protein therapies, most of which are injectables, have several limitations. Large proteins such as monoclonal antibodies (mAbs) suffer from poor absorption after subcutaneous injections, thus forcing their administration by intravenous injections. Even small proteins such as insulin suffer from slow pharmacokinetics which poses limitations in effective management of diabetes. Described herein is a deep eutectic-based formulation that offers a generalized strategy for improving protein absorption after subcutaneous injections. This lead formulation enhanced absorption of mAbs after subcutaneous injections by ˜200%. The same composition also enabled systemic absorption of subcutaneously injected insulin faster than Humalog, the current gold-standard of rapid acting insulin. Mechanistic studies reveal that the beneficial effect of deep eutectics on subcutaneous absorption is mediated by their ability to reduce the interactions of proteins with the subcutaneous matrix, especially collagen. Studies also confirmed that our deep eutectic formulations are safe for subcutaneous injections. Deep eutectic-based formulations described here open new possibilities for subcutaneous injections of therapeutic proteins.

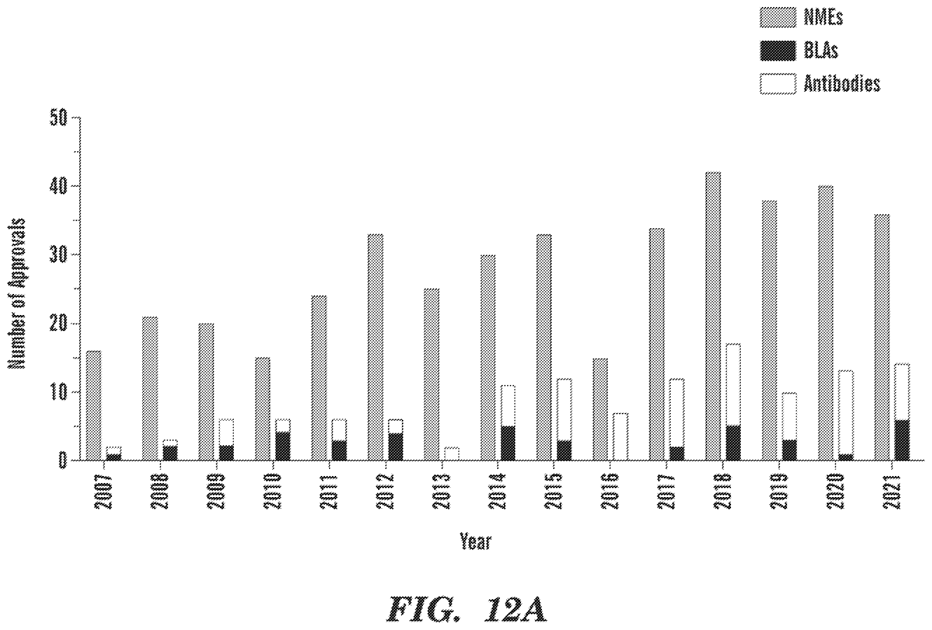

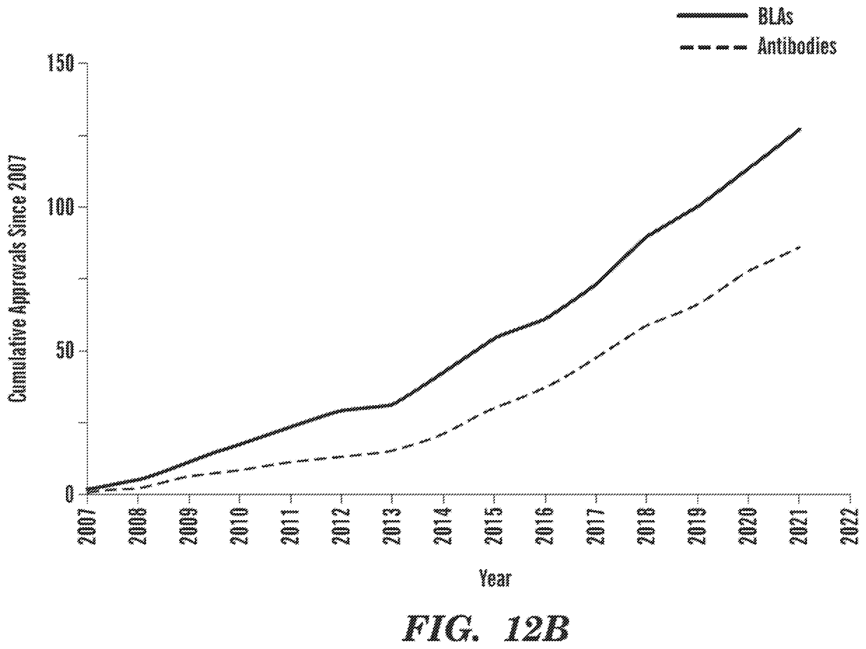

Recombinant protein biologics are among the most extensively used therapeutics in the clinic over the past 40 years. In the last 15 years alone, 127 therapeutic proteins including 86 monoclonal antibodies (mAbs) or antibody conjugates, were approved and they account for nearly 25% of total FDA-approved therapeutics (FIG. 12A). The rate of approval of biologics has further accelerated over the last few years (FIG. 12B).¹ Delivery logistics of biologics, on the other hand, have seen limited innovation. Many mAbs are delivered by intravenous (IV) administration despite their obvious limitations. Subcutaneous administration (SC) offers a better alternative to IV delivery since it converts an hours-long, clinic-based IV infusion into a quick injection at home. This lowers the treatment cost and reduces the strain on healthcare resources.^{2,3} SC injections also reduce the pain and they decrease the likelihood of infection, especially sepsis, compared to IV administration.⁴ These benefits, coupled with the potential for self-administration, favor the use of SC administration over IV administration, leading to enhanced patient compliance and better disease management.⁵⁻⁷ Despite these advantages, use of subcutaneous injections for mAbs is limited by their poor bioavailability after subcutaneous injections. Subcutaneously administered biologics must traverse the subcutaneous tissue, compromising cellular milieu including adipocytes, fibroblasts, and immune cells, and extracellular matrix (ECM) proteins including collagen, elastin, and fibronectin⁸ before reaching the systemic circulation by intravasation into the local blood or lymph capillaries. Smaller biologics, e.g., insulin (MW˜6 kDa) drain into blood capillaries whereas larger proteins such as mAbs (MW˜150 kDa) drain into lymph capillaries.⁹

Insulin and mAbs have been at the center of attention for development of methodologies to improve absorption after their subcutaneous injection. These efforts can be classified into two groups; protein modification and formulation engineering. Substantial efforts have been focused on developing new insulin analogs to control the duration of its action over a long, intermediate, short, or rapid time scales. Many academic research efforts have been focused on developing strategies for sustained insulin release, however, rapid-acting insulin formulations remain relatively underexplored.¹⁰ Rapid-acting insulin is especially significant for diabetes management since it can mitigate hyperglycemic episodes by decreasing the time between blood glucose measurements and insulin's systemic effect. This is particularly beneficial for continuous infusion pumps with a closed feedback response.¹¹ A clinically approved insulin analog, Humalog (insulin lispro) exploits engineering of insulin's sequence to maintain its monomeric structure and induce rapid absorption.¹² Targeted amino acid mutation decreases the stability of insulin-oligomer, i.e., dimers and hexamers, thereby shifting the equilibrium to monomers which can be readily absorbed into systemic circulation.¹³ Protein engineering-based approaches have also been attempted for improving mAb pharmacokinetics. For example, modifications of the Fc region have been attempted to increase mAb's subcutaneous absorption.^{14,15}

While protein engineering-based strategies offer the advantage of building pharmacokinetics into protein design, they also suffer from the design constraints and they often require a compromise between protein's biological activity and pharmacokinetics. Formulation engineering, on the other hand, offers an alternate approach to control protein pharmacokinetics. These efforts have been based on two principles: reduced protein aggregation and enzymatic matrix degradation in the subcutaneous compartment. Mann et al.¹⁶ developed a polymer excipient that reduced insulin aggregation and decreased the time to insulin peak in vivo after subcutaneous injections. These studies reported 64% faster insulin absorption compared to Humalog. Poly(ethylene glycol) (PEG)¹⁷⁻¹⁹ and Trehalose glycopolymer²⁰ conjugation have also been used to stabilize insulin monomers for rapid-acting formulation but can have negative effects on pharmacokinetics.

While a number of antibody delivery systems have been described in the literature, many are focused on sustained release and that too in places other than the subcutaneous space.²¹ As such, efforts to improve systemic absorption of mAbs after subcutaneous injection have focused less on improving bioavailability and more on pushing formulation concentration and injection volume limits.²² One of the few exceptions is the use of recombinant hyaluronidase, an ECM-degrading enzyme, co-formulated with mAbs for improving bioavailability.²³ Five hyaluronidase-based mAb products have received FDA approvals: Rituxan HYCELA, Herceptin HYLECTA, DARZALEX Faspro, PHESGO.

Described herein is the use of biocompatible deep eutectics to improve subcutaneous pharmacokinetics via a novel mechanism of action which adds a new tool towards improving subcutaneous formulations for biologics. Unlike many other biological barriers, transport barriers in the subcutaneous space are poorly understood. The subcutaneous ECM proteins in the extracellular space, of which Type I collagen is the most abundant,²⁴ pose a barrier to absorption due to nonspecific binding interactions with the injected biologics.²⁴ Subcutaneously injected protein formulations interact with the ECM proteins and such interactions lead to delayed or reduced absorption into systemic circulation. We have previously shown that ionic liquids (ILs) and deep eutectic solvents (DESs) have the potential to reduce the interactions of a broad variety of serum proteins with substrates²⁵. Taking advantage of this unique ability, the inventors hypothesized that ionic liquids and deep eutectics can also mitigate the interactions of injected proteins with the proteins in the subcutaneous ECM matrix. This approach is referred to herein as Subcutaneous Protein Administration using Deep Eutectics (SPADE). Described herein is the screening of SPADE formulations and their ability to improve subcutaneous injection speed and bioavailability for insulin and Rituximab, respectively. The present studies also demonstrate the lead SPADE formulation reduces protein interactions with collagen and is safe to inject as evidenced by repeat dose administration.

### Results

### Insulin Stabilization Using DES

Ten DESs were synthesized using a salt metathesis reaction at the cation to anion ratios of 1 to 2 as previously described.²⁶ (see also International Patent Publications WO 2019/099837; WO 2020/180534; WO 2020/205409; and WO 2021/102084 each of which is incorporated by reference herein in its entirety). Compositions with a cation:anion ratio of 1 are referred to as ILs and those with an asymmetric ratio are referred to as DESs. These DESs were designed to exhibit a range of chemical properties, especially hydrophobicity as this parameter is expected to be a key determinant of their ability to impact transport and subsequent absorption in the subcutaneous tissue. Choline and acetylcholine were investigated as the two cations because choline has been a commonly explored cation in previous studies of ionic liquids²⁵⁻³². Aetylcholine is a more hydrophobic cation. These cations were subsequently paired with five anions, glycolate, lactate, propionate, hexenoate, and geranate, that covered a spectrum of molecular weights, carbon chain lengths, and hydrophobicities (Table 4). After synthesizing the library of ten DESs (abbreviations in Table 5), 1D proton nuclear magnetic resonance (NMR) was used to confirm their structures (data not shown). The ten DESs were then formulated as a 0.5% solution in sterile saline, which readily solubilized 100 U/mL regular insulin (commonly used concentration in clinical formulations) at neutral pH.

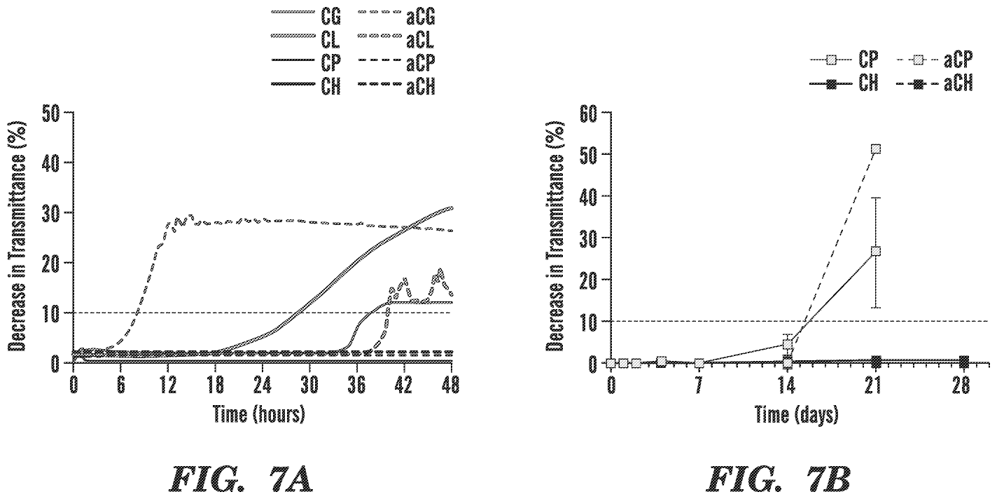

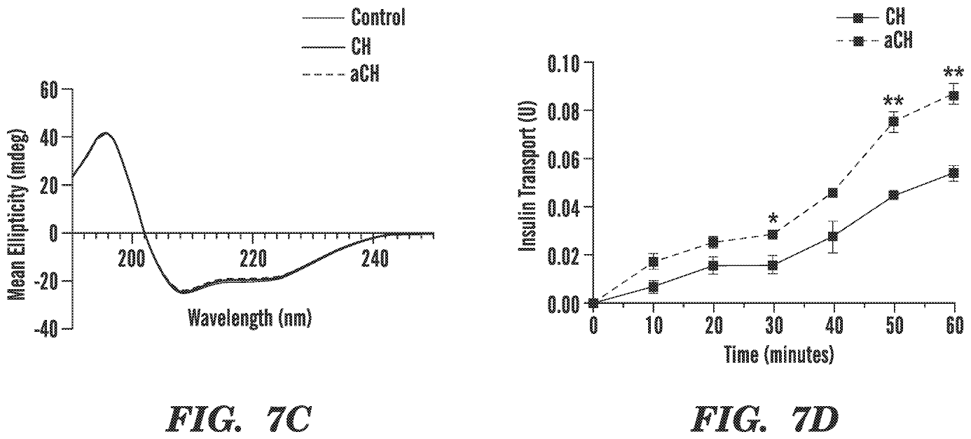

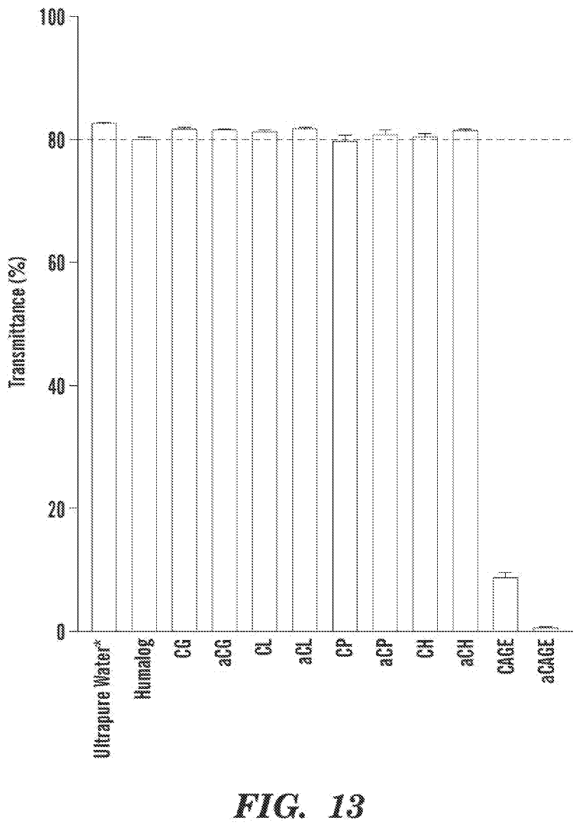

Successful dissolution of insulin was determined by the turbidity of the formulation, assessed by converting absorbance at 540 nm to transmittance.¹⁶ The percent transmittance threshold for the successful formulation was set at 80% as this was the mean transmittance for the clinical comparator (Humalog). Low transmittance values are indicative of insoluble insulin aggregates. All but two formulations met this criterion (FIG. 13). To further screen the remaining DES formulations, a stressed aging test was performed to determine their stability for 50 hours at 37° C. with constant shaking. A decrease in transmittance of greater than 10% at any point during the 50-hour period was seen for CG, aCG, CL, and aCL (FIG. 7A) and these formulations were excluded from further experiments, leaving four viable formulations for further experimentation (CP, aCP, CH, and aCH).

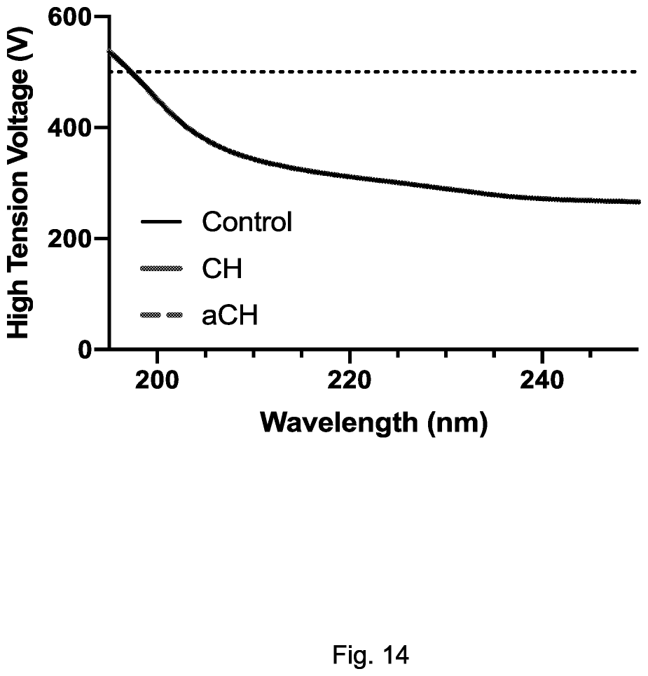

The final screening was performed to assess cold-chain stability and confirm monomer conformational stability. Cold-chain, namely refrigeration between 2 and 8° C., is critical for protein biologic stability to prevent unfolding, aggregation, and is widely used to extend shelf-life.³³ Among CP, aCP, CH, and aCH formulations incubated at 4° C. over 28 days, both propionate-based DES formulations experienced aggregation based on a greater than 10% decrease in transmittance between Day 14 and 21, while both hexenoate-based DESs were stable through Day 28 (FIG. 7B). The stability of hexenoate-based formulations was further confirmed by assessing the protein's secondary structure through circular dichroism (CD). The CD spectra of insulin incubated with the two leading formulations, CH and aCH, at 37° C. for two hours matched that of the control insulin dissolved in sodium phosphate buffer, indicating that neither DES formulation induced notable protein unfolding at physiological temperature and pharmacologically relevant time scales (FIG. 7C). CD spectra integrity was also confirmed by measuring the high-tension voltage, which was maintained below 500 V for the relevant insulin CD wavelengths (FIG. 14).

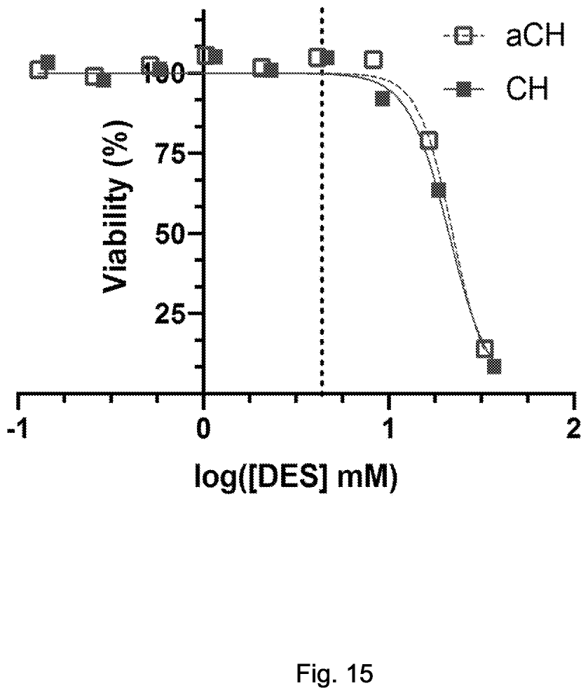

To further select the formulation from two leads, CH and aCH, their effect on trans-endothelial transport of insulin was measured in vitro. Endothelial cells of blood capillaries provide a barrier to vascular drainage and a beneficial effect of DESs on trans-endothelial transport can further improve pharmacokinetics. To assess this possibility, in vitro transport of insulin across human umbilical vein endothelial cell (HUVEC) was measured using transwell permeability assay. 0.15% v/v or approximately 4.3 mM DES was used in these studies based on the in vitro tolerability study (FIG. 15). HUVEC monolayers were formed on gelatin-coated transwell and confirmed by fluorescent imaging with Hoescht 33342 (nuclei) and Actin 488 (cell cytoskeleton) (Data not shown). aCH exhibited enhanced vascular permeability compared to CH (FIG. 7D). The combination of stability and transport experiments led to the choice of aCH as the lead composition for subsequent studies.

### SPADE Prevents Interactions Between Therapeutic Proteins and ECM Collagen

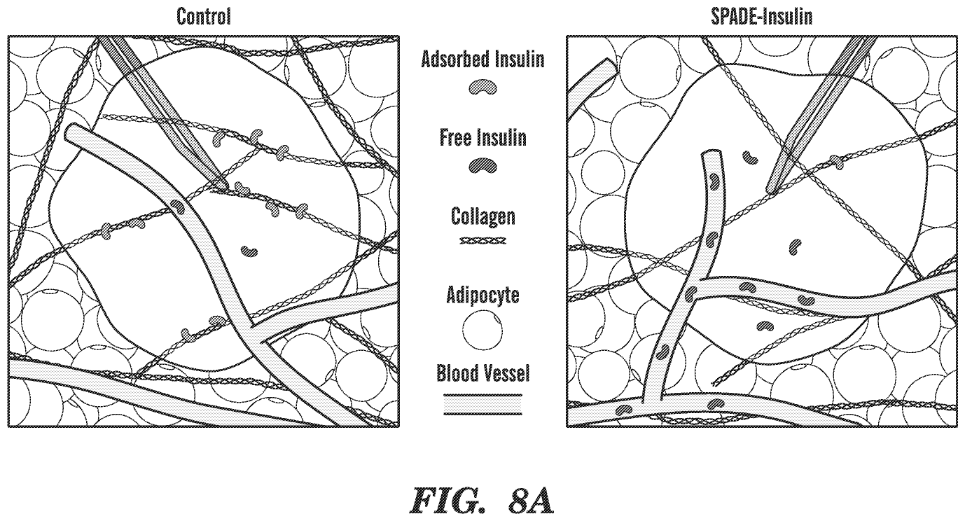

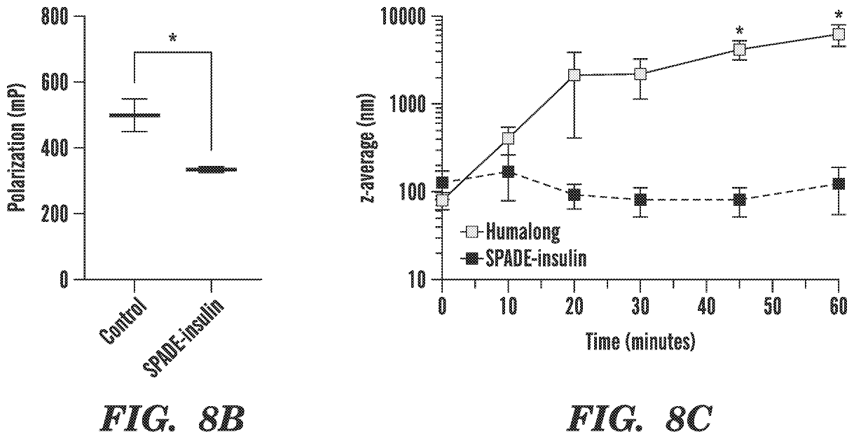

The ability of SPADE to mitigate the interactions of proteins with ECM was assessed using the lead DES, aCH. A schematic representation of the hypothesized mechanism is depicted in FIG. 8A. SPADE (right) is hypothesized to decrease non-specific binding between administered therapeutic proteins and subcutaneous extracellular proteins such as collagen to allow for faster and greater absorption of protein biologics. To test this hypothesis, fluorescence polarization (FP) was performed and dynamic light scattering (DLS) utilized to determine the degree of collagen-insulin association. FP is commonly used as a ligand-binding assay to analyze interactions between small molecules or peptide payloads binding to their protein targets,^{34,35} based on the principle that a protein-bound, fluorescently-labeled molecule rotates more slowly and emits in the parallel direction, resulting in a higher polarization value.³⁶ FP was performed immediately after mixing Cy5.5-labeled insulin, either formulated in aCH or saline (control), and Type I human collagen. The resulting polarization values showed that aCH inhibits non-specific insulin-collagen binding compared to the control (FIG. 8B). The average polarization was 1.5 times higher for the control group than the SPADE group. To further confirm the FP results and compare against Humalog, DLS was used to measure the average particle diameter (z-avg) after mixing insulin formulations with collagen, as the z-avg for insulin-bound collagen would increase with fewer unbound insulin molecules remaining and only the collagen or collagen-insulin complex being detected. The initial average z-avg for both the Humalog- and SPADE-insulin-collagen mixtures were approximately 100 nm. The Humalog group exhibited an increase in z-avg for the entire time course, reaching a value exceeding 5000 nm over 60 mins. In contrast, SPADE-insulin group remained approximately at 100 nm over the same time period (FIG. 8C). The results from FP and DLS strongly support that the SPADE reduces non-specific binding between insulin molecules and collagen that is ubiquitously present in the subcutaneous space as part of the ECM.

### SPADE Enhances Insulin Pharmacokinetics

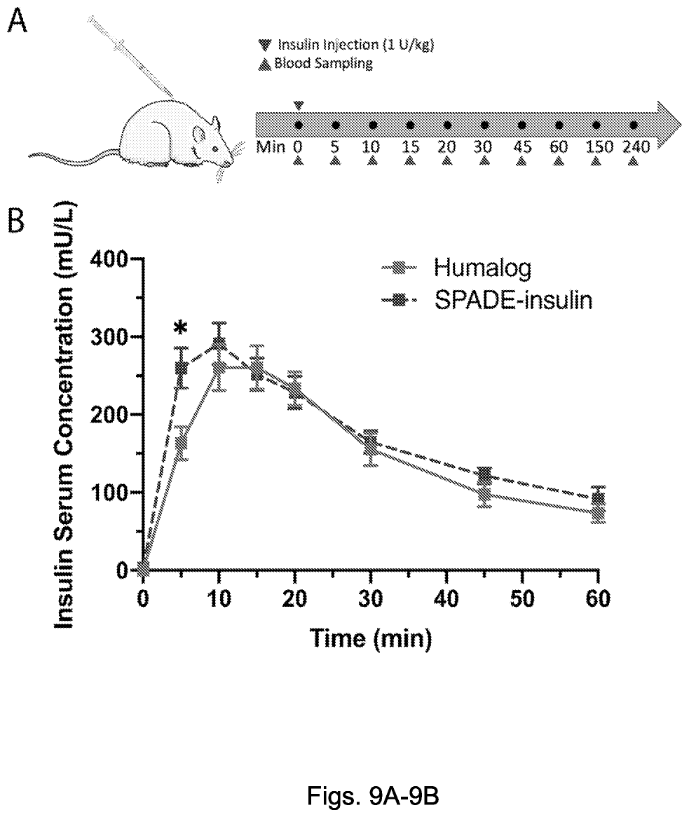

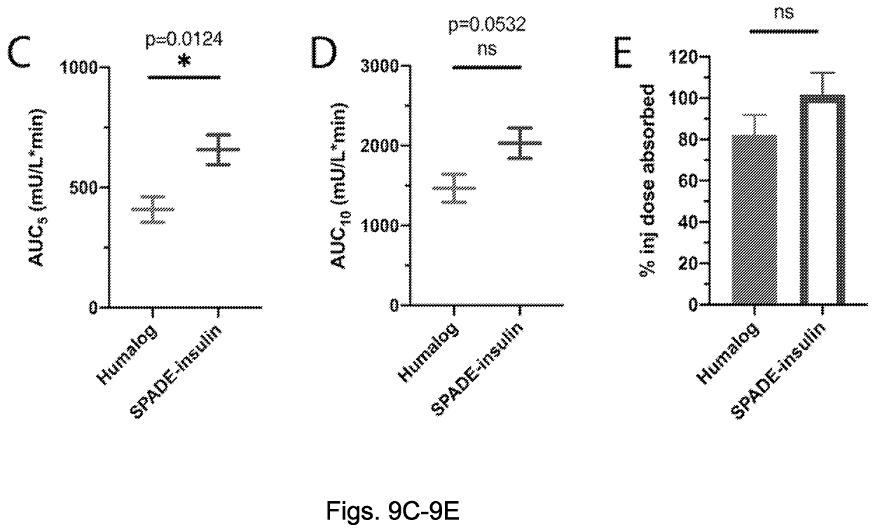

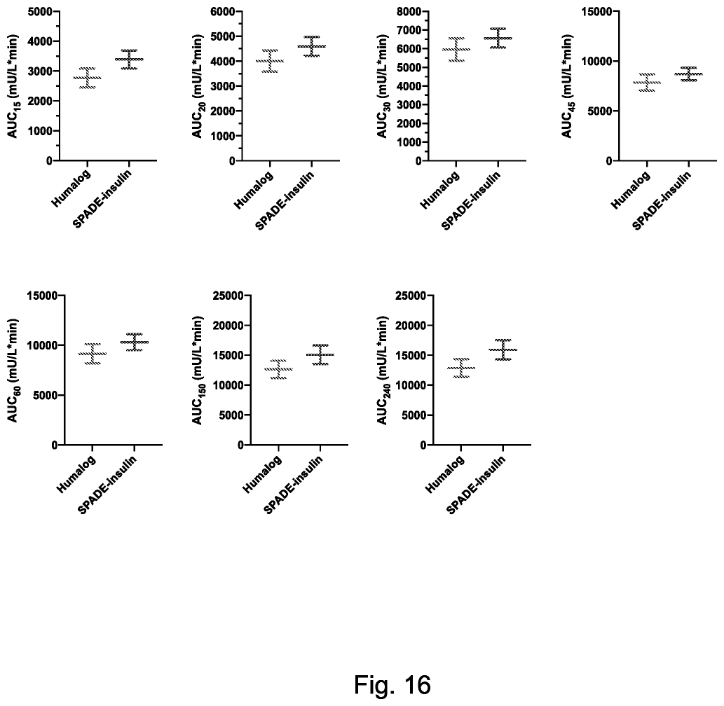

Pharmacokinetics of 1 U/kg SPADE-insulin was compared against the equivalent dose of Humalog, the clinical gold standard of fast-acting insulin analog, in rats. The study design is detailed in FIG. 9A. SPADE-insulin significantly accelerated insulin absorption from the subcutaneous tissue compared to Humalog (FIG. 9B), as shown by 1.6-fold higher serum level of insulin at 5 minutes after injection of SPADE-insulin compared to Humalog. In addition, the area under the curve (AUC) was calculated at each time point to quantify the accumulation of insulin in the bloodstream. The cumulative AUC also increased significantly at 5 minutes by 1.6-fold in SPADE-insulin-treated rats compared to Humalog-injected rats (FIG. 9C). While the cumulative AUC remained higher in the SPADE-insulin treated group than the Humalog-treated group at 10 minutes (FIG. 9D) and for the extent of the study (FIG. 16), statistically significantly higher absorption of insulin at an early time point of 5 min suggests that aCH in SPADE-insulin plays an important role immediately after the administration and could benefit patients in need of ultra-fast insulin that ensures less time delay to therapeutic effect. It was also found that the bioavailability expressed as the percent of the injected dose was higher for the SPADE-insulin group than the Humalog group, although not statistically different (FIG. 9E).

### SPADE is Non-Toxic and Safe Upon Repeat Dosing

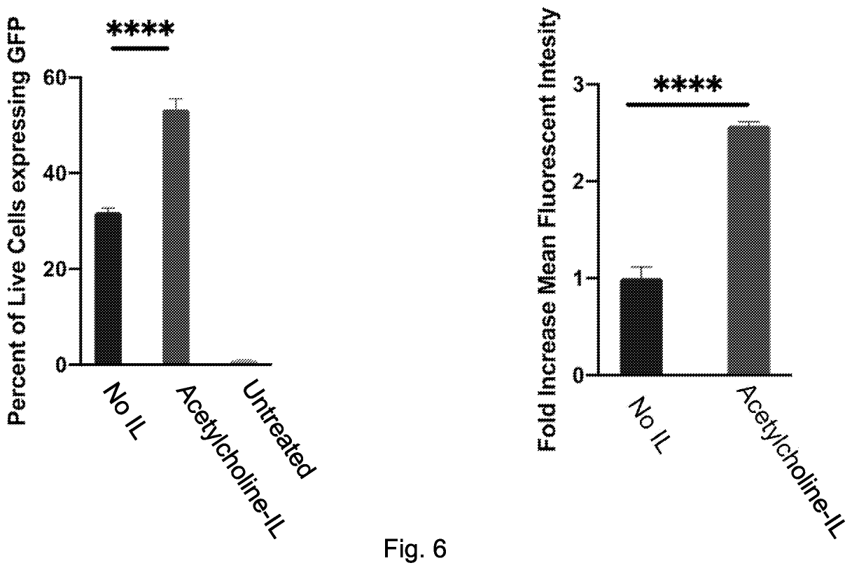

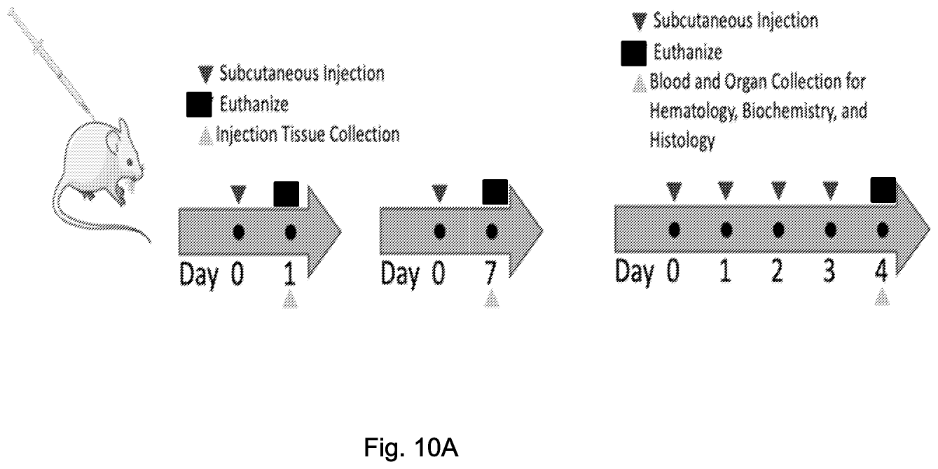

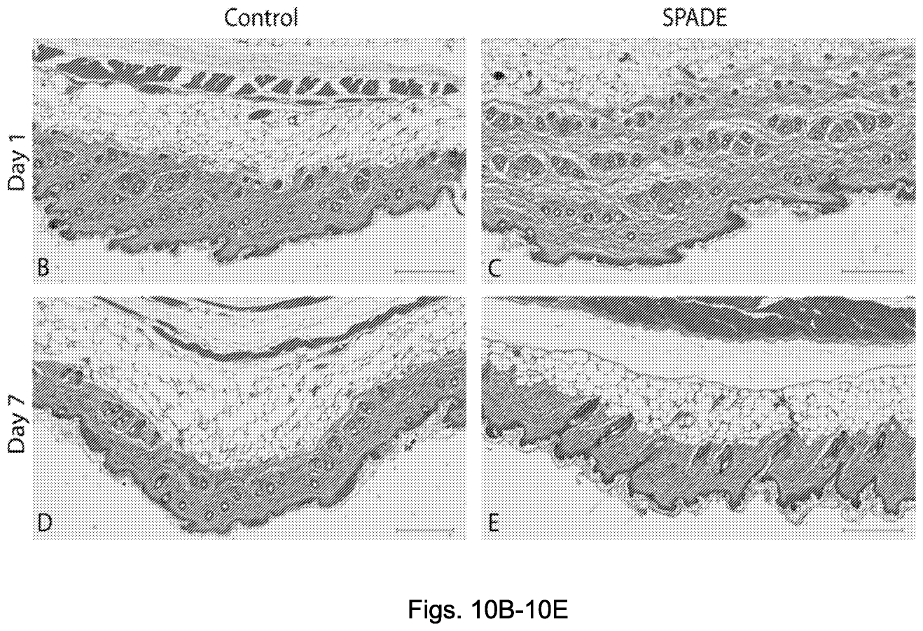

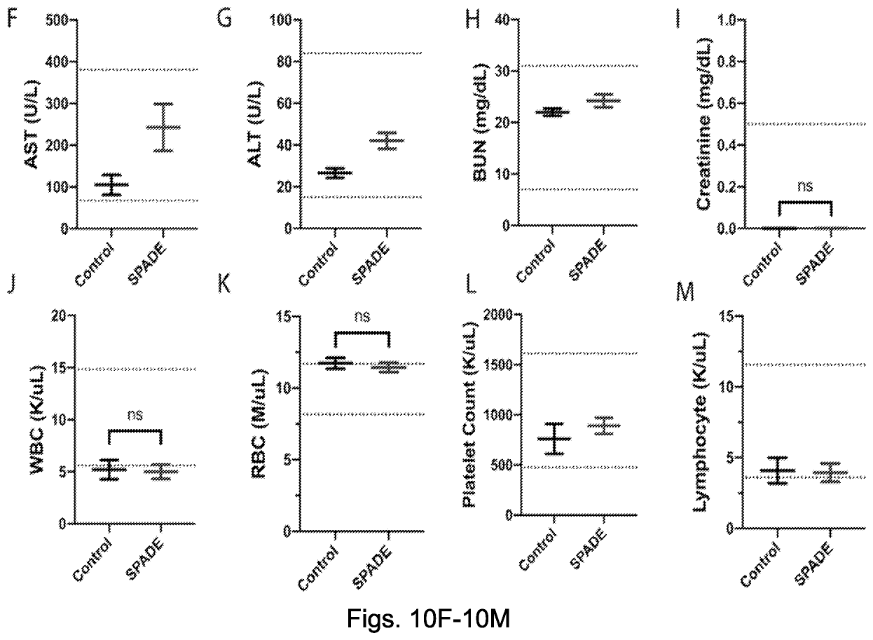

Two different studies were performed using saline (control) and SPADE-alone dosed BALB/c mice to examine local (injection site) and systemic toxicities. The first study used four cohorts to examine injection site toxicity 24 hours and 7 days following subcutaneous dosing using hematoxylin and eosin staining (H&E) (FIG. 10A). There was no notable difference between the injection sites of the two formulations for either time point (FIGS. 1B-10E). There is no noticeable difference between hematoxylin staining or nuclei pattern when comparing SPADE and control injection site sections.³⁷ Additionally, a blind analysis of the tissue samples by a histopathologist confirmed no signs of toxicity, with respect to inflammation, edema, necrosis, fibrosis, or degeneration.

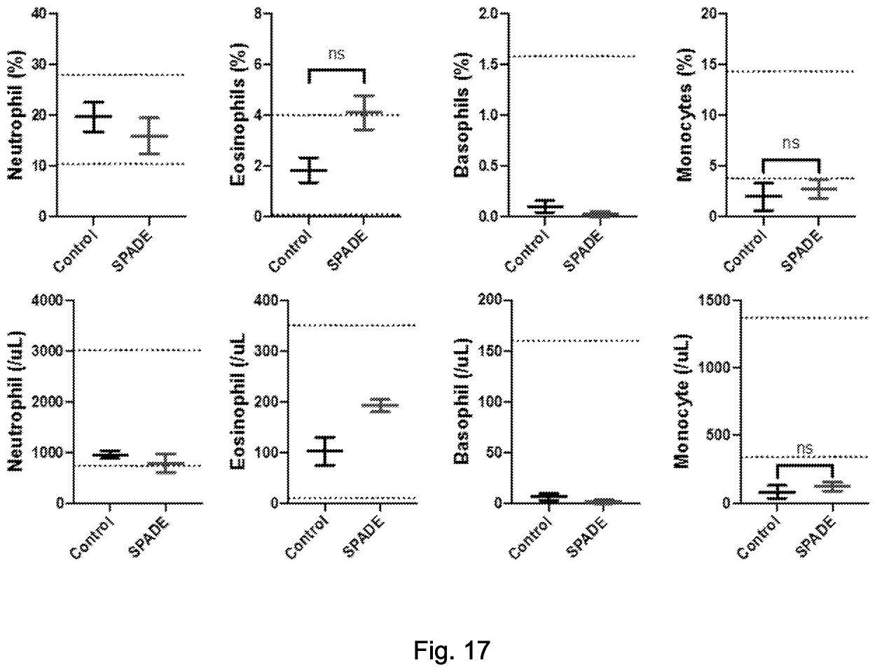

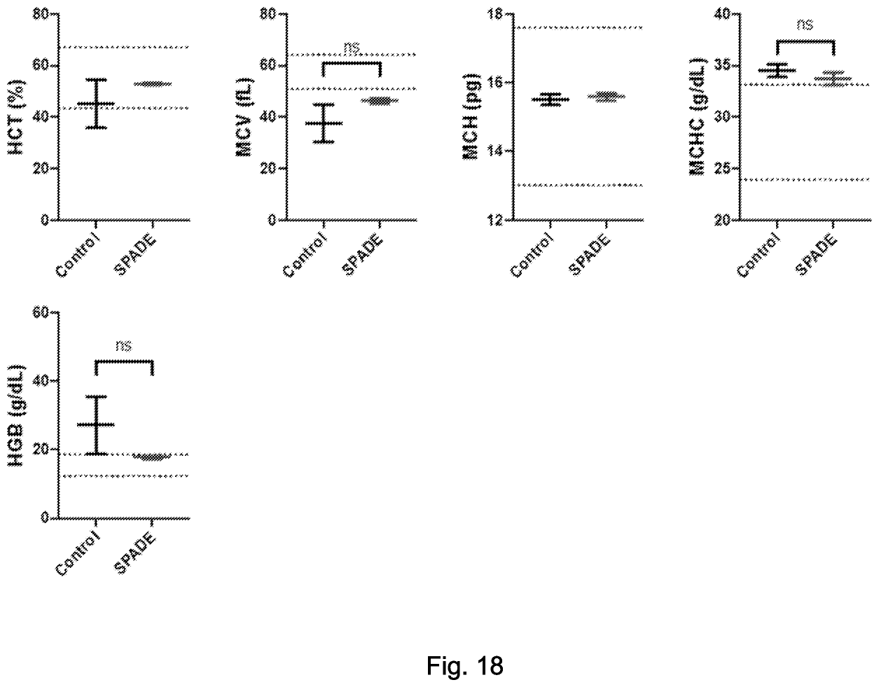

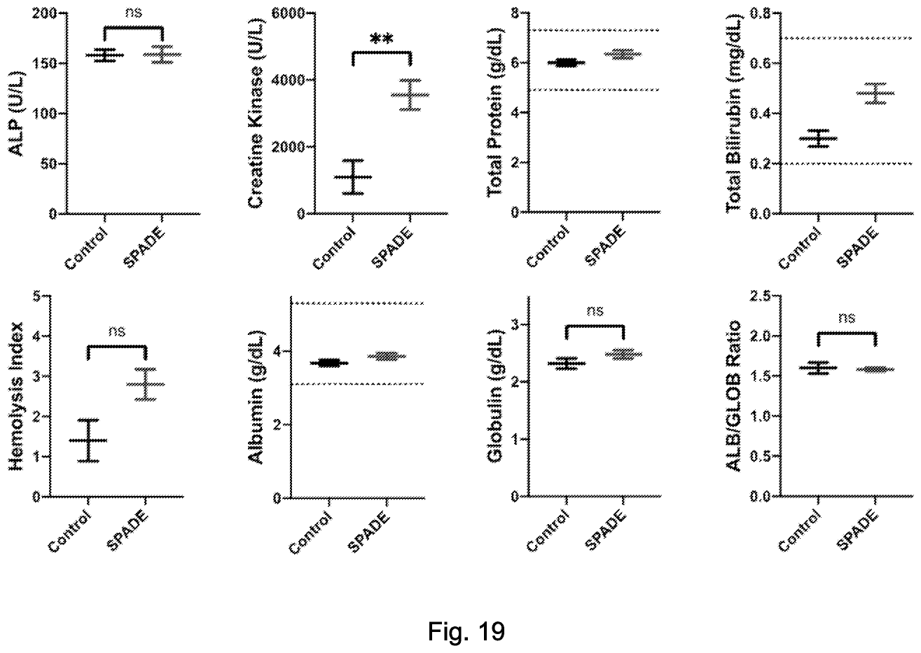

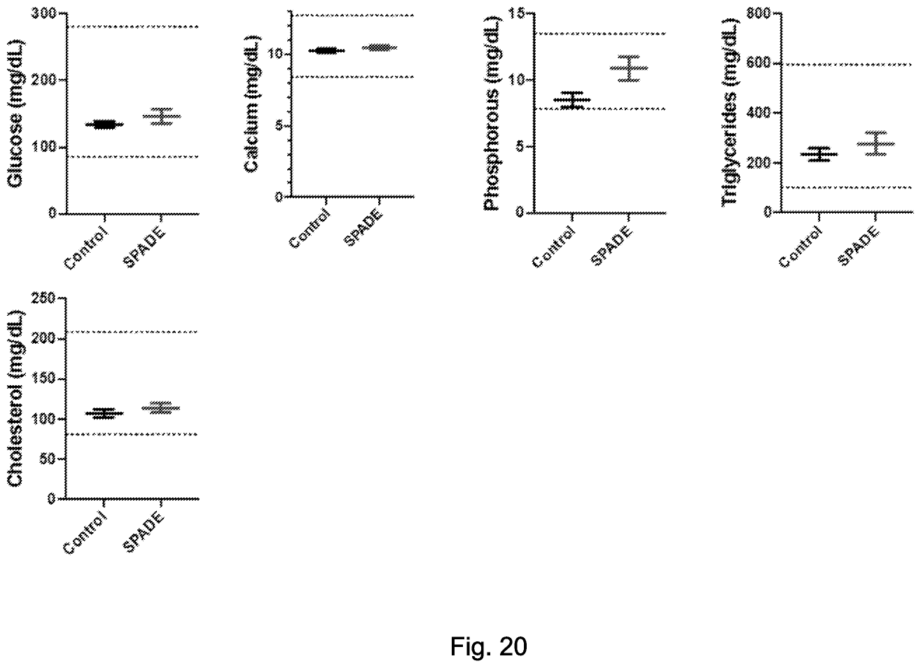

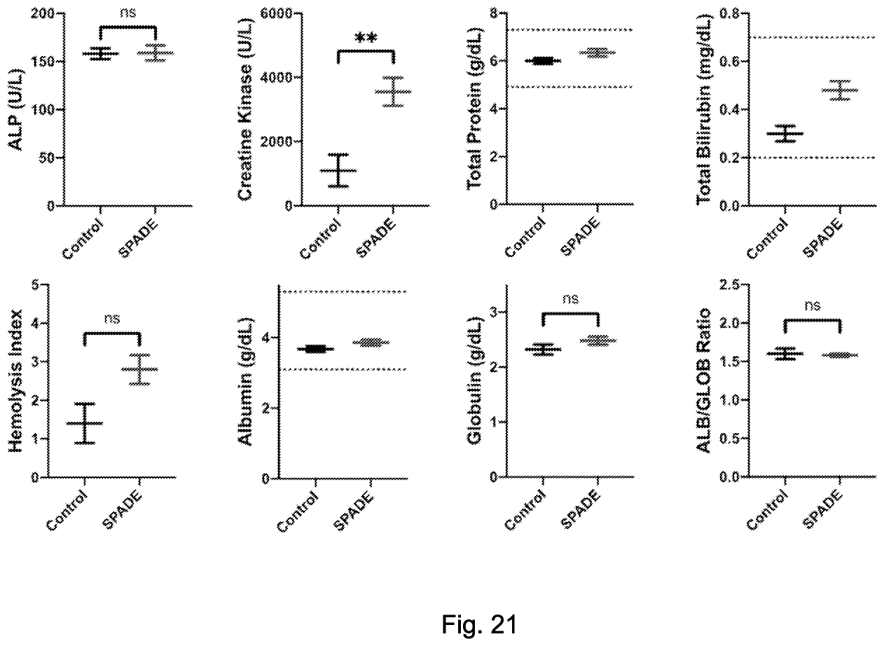

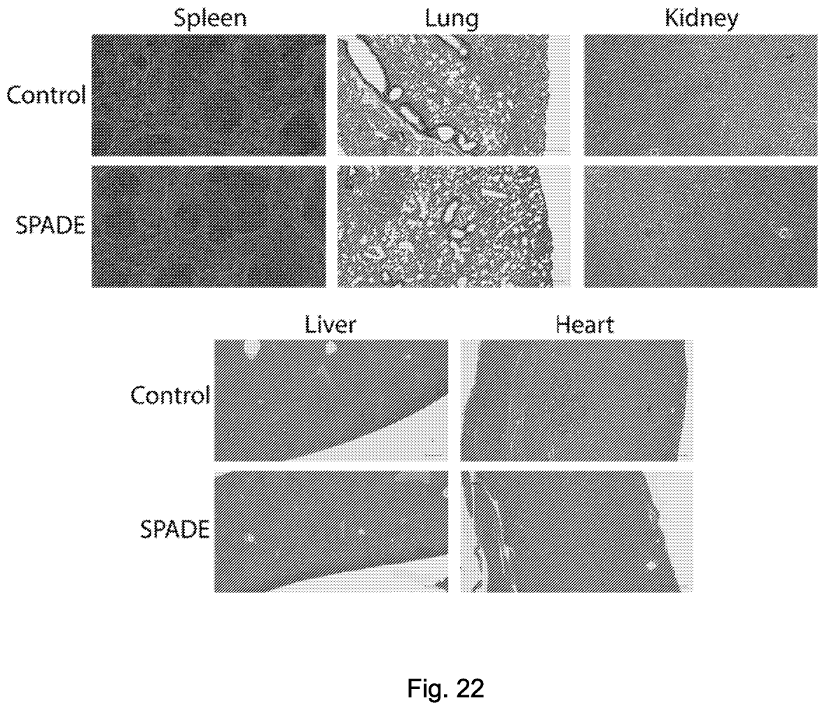

The inventors then examined systemic toxicity from repeat dosing with SPADE. Blood and major organs were analyzed 24 hours after the last of four daily subcutaneous injections of saline (control) and SPADE alone formulations. Two key liver function markers, aspartate transaminase (AST) and alanine transaminase (ALT), were within the accepted range (FIGS. 10F-10G).³⁸ AST and ALT are both transfer enzymes found in hepatocyte cytoplasm that are released into the serum upon hepatocyte damage, thus levels elevated outside the expected range indicated improper liver function.³⁹ Additionally, two key kidney function markers, blood urea nitrogen (BUN) and creatinine, were also within the accepted range for female BALB/c mice (FIGS. 10H-10I).³⁸ Urea and creatinine are both filtered out of serum by the glomeruli of the kidneys, thus BUN and creatinine serum levels are markers for glomerular filtration rate and elevated levels indicate kidney dysfunction.⁴⁰ Other biochemical markers for SPADE-treated mice were either within the accepted range or not statistically different from the saline control (FIGS. 17-18). Whole blood analysis showed that SPADE is well-tolerated and that no significant systemic immune response occurred upon repeat dosing. White blood cell, red blood cell, platelet, and lymphocyte counts were all within the accepted range or ones that were outside were not statistically different from the saline control (FIGS. 10J-10M). Other peripheral cells measured in this study were within the accepted range or not statistically different from the saline control (FIGS. 19-21). Systemic toxicity was also assessed by histopathology of mice who received repeat SPADE dosing. H&E staining showed no marked difference between SPADE and control mice in vital organs, including spleen, lung, kidney, liver, and heart (FIG. 22). These finds were also confirmed by a blind analysis by a histopathologist.

### SPADE Enhances Bioavailability of Rituximab

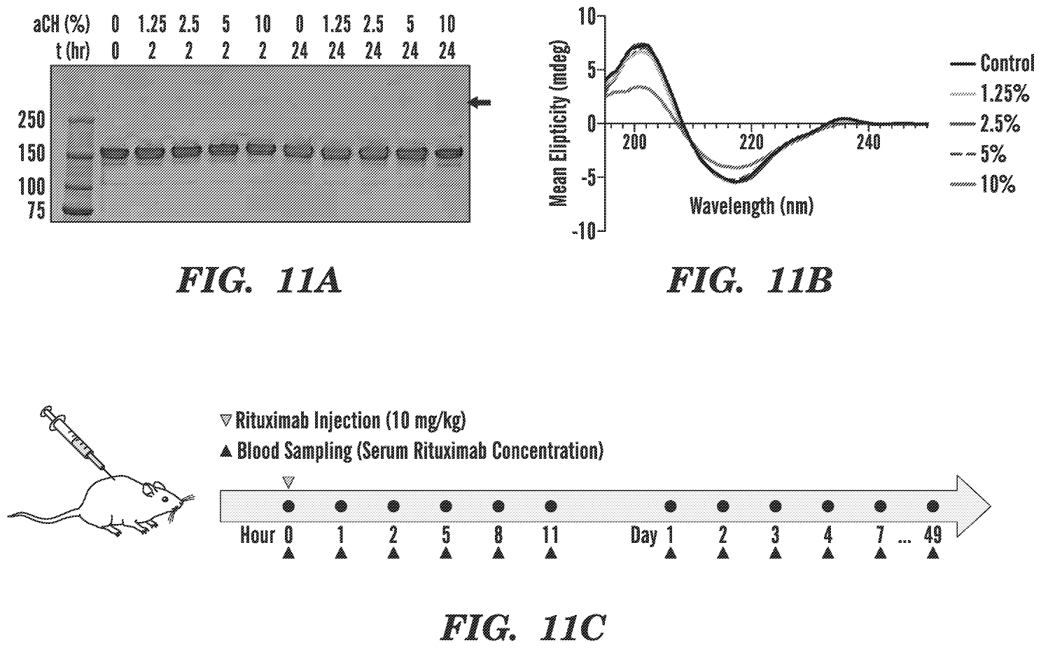

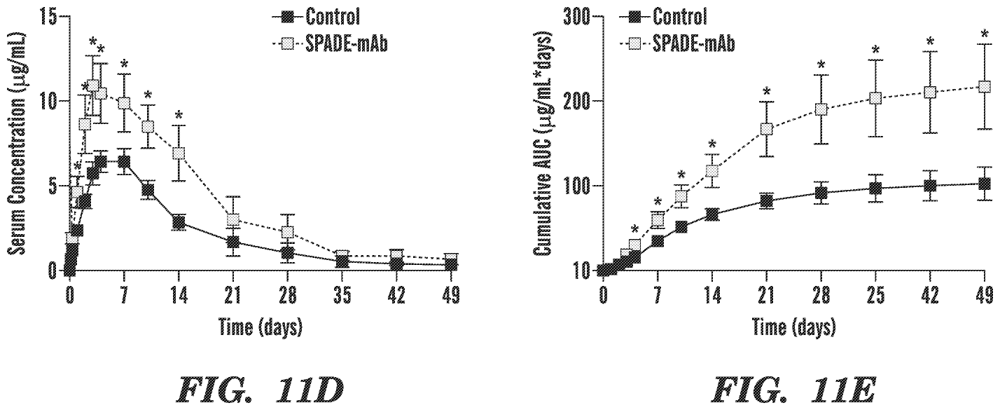

Described herein the exploration of whether SPADE can also enhance the absorption of a large protein biologic, namely monoclonal antibody rituximab (SPADE-mAb), that would benefit from greater absorption from subcutaneous administration. Due to the clinical requirement of higher dosing for subcutaneous antibody formulation compared to insulin, we first assessed DES concentration for stable SPADE formulation with rituximab using SDS-PAGE and CD. Formulations were prepared with various DES concentrations and consistent rituximab concentrations and incubated at 37° C. for 2 and 24 hours. After incubation, the samples were dialyzed against sodium phosphate to remove DES, and the resulting samples were diluted to the same concentration via UV-Vis spectrophotometry before SDS-PAGE and CD analyses. We determined that all DES formulation concentrations were stable apart from 10% (v/v) DES, as there is a faint band further up the gel that is indicative of higher-order molecular weight species from potential aggregation (FIG. 11A). To further confirm this CD was used to assess the secondary structure of the samples that were incubated for 24 hours. The CD results were consistent with the SDS-PAGE results as the 10% (v/v) DES formulation saw a shift in mean ellipticity when compared to the control, while none of the other DES concentrations experienced this shift (FIG. 11B). This shift is indicative of misfolding that likely caused the aggregation seen in the SDS-PAGE gel. These stability studies indicated 5% (v/v) aCH as the appropriate concentration for the SPADE-mAb formulation.

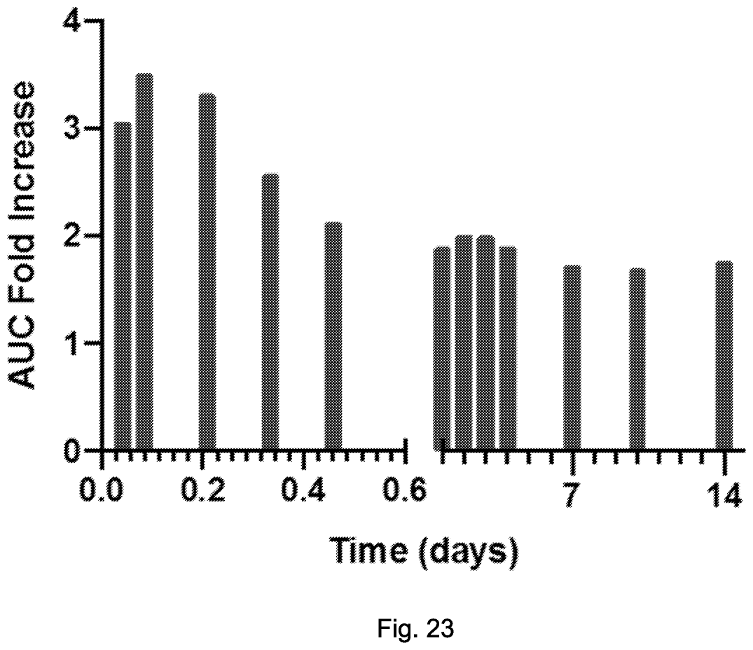

Following the experimental design shown in FIG. 11C, pharmacokinetics study was performed and it serum rituximab levels were quantified by ELISA. Serum rituximab levels for SPADE were significantly higher 1 hour after administration and remained so through day 14, apart from 11 hours and 7 days (FIG. 11D). Average peak rituximab serum concentration was higher, 10.89 compared to 6.41 ug/mL, and occurred earlier, day 3 compared to day 7, for the SPADE-mAb group than the control. AUC was also statistically higher at all time points of the study (FIG. 11E). The significance values for each timepoint can be found in Table 6. At the study endpoint, AUC for SPADE treated group was 217.11 mg/mL*day, which is 2.12 times higher than the control. AUC also had a higher fold increase over the control at early time points compared to later time points, indicating that SPADE-mAb is also improving the early absorption kinetics of antibodies as in the case of SPADE-insulin (FIG. 23).

### Discussion

Described herein is the development of a deep eutectic solvent-based formulation strategy, SPADE, to improve subcutaneous delivery of therapeutic protein biologics. It is demonstrated that SPADE: i) can be stably formulated with insulin and antibodies, ii) is a safe, non-toxic formulation strategy, and iii) improves pharmacokinetics of insulin and the bioavailability of monoclonal antibodies by preventing non-specific binding interactions between therapeutic proteins and collagen ECM proteins. SPADE utilized a deep eutectic solvent that can be synthesized in a facile manner, allowing for a simple and scalable formulation. SPADE is prepared from biocompatible ions with known history of human exposure, thus improving its safety profile.

Fast-acting insulin reduces the time between blood glucose measurement and insulin's systemic effect, and it improves the ability to maintain euglycemia. This could make a significant difference especially for patients who use continuous subcutaneous insulin infusion (insulin pumps) that allows blood glucose level within the target ranges for a higher percentage of the day.¹¹ While insulin analogs have been used to improve absorption kinetics in the clinic, significant research in academia has also focused on polymer¹⁶ and polypeptide⁴¹ excipients, polymer conjugation,¹⁹ and protein co-formulation.⁴² While these strategies have shown success in vivo, DESs and ILs offer an appealing alternative since they can easily be manufactured at scale with fewer steps and lower costs to allow for simpler fast-acting insulin formulation.

Beyond reducing protein-collagen interactions, SPADE formulations have the potential to decrease protein-protein interactions within the formulation. This is perhaps less critical for therapeutic proteins like insulin where the clinical formulations are relatively dilute (e.g., 3.47 mg/mL or 100 U/mL). However, such interactions can play a critical role in mAb formulations which deploy a much higher concentration, e.g., greater than or equal to 100 mg/mL. SPADE can provide a novel tool to reduce mAb-mAb interactions and improve stability of mAb formulations.

SPADE increased rituximab absorption (measured as AUC) by 112% over saline formulated rituximab. In a study performed by Kagan et al.,²³ rats dosed dorsally with 10 mg/kg of rituximab, hyaluronidase was used to increase rituximab absorption by 91%. This high efficacy of SPADE supports its potential clinical use since similar hyaluronidase is already in the clinical use for multiple mAbs. Further, SPADE demonstrated the ability to increase absorption over a wide range of protein biologic sizes, 5.8 to 150 kDa, and thus can offer a subcutaneous formulation strategy for other protein biologics, peptides, and nucleic acids.

### Materials and Methods

### Materials

Choline bicarbonate, acetylcholine chloride, lactic acid, glycolic acid, propionic acid, trans-2-hexanoic acid, geranic acid, regular lyophilized insulin, gelatin powder, Millicell Transwell inserts, hydrochloric acid, and sodium hydroxide were all purchased from Sigma Aldrich (St. Louis, MO). 0.2 M sodium phosphate buffer was purchased from Boston BioProducts, Inc. (Milford, MA). HUVEC cells, growth media, and additives were purchased from ATCC (Manassas, VA). CellTiter 964 Aqueous One Solution Cell Proliferation Assay (MTS) was purchased from Promoega (Madison, WT). Type I Human Collagen was purchased from Advanced BioMatrix (Carlsbad, CA). Cy 5.5 fluorescently-labeled insulin was purchased from Nanocs Inc. (New York, NY). SDS-PAGE Ladder (Precision Plus Protein™ All Blue Prestained Protein Standards #1610373), lamelli buffer, Tris/Glycine/SDS Running Buffer, Coomassie blue stain, Mini-PROTEAN Tetra Vertical Electrophoresis Cell, and power supply were all purchased from Bio-Rad Life Sciences (Hercules, CA). Rituximab Biosimilar was purchased from BioXCell (Lebanon, NH). Insulin ELISA and Insulin Lispro NL-ELISA were purchased from Mercodia Inc. (Winston Salem, NC). Rituximab ELISA was purchased from Eagle Biosciences (Amherst, NH).

### Methods

Deep Eutectic Solvent Synthesis and Formulation Preparation. Deep eutectic solvent were synthesized as previously described.^{26,32} Briefly, weak acids of desired anions were dissolved in minimal ultrapure water in a round bottom flask. The solution was heated, with stirring, using an oil bath to 40° C. in the case of the choline-based DESs and 65° C. in the case of the acetylcholine-based DESs. To prevent round bottom flask over-flow, the cationic precursor was slowly added at a ratio of 1:2 cation: anion. The mixture was allowed to fully react overnight. Excess water was removed first by Rotary Evaporator for 3 hours then by vacuum oven set to 60° C. for 2 days. The structure was then confirmed using nuclear magnetic resonance (Bruker AVANCE NEO 400).^{25,32,43} To prepare insulin formulations, lyophilized insulin was suspended in saline. Sufficient DES was added and in cases where this addition did not dissolve insulin, 1 M hydrochloric acid was added to adjust the pH to 2.5-3 and the solution became clear. To adjust the pH to between 7.0-7.5, 1 M sodium hydroxide was added. The formulation was adjusted by adding saline to give final insulin and DES concentrations of 100 U/mL and 0.5% (v/v), respectively. To prepare antibody formulations, ˜40% (v/v) solutions of DESs in 0.9% saline were pH adjusted to 7.0-7.5 using 10M sodium hydroxide. The DES solution was then formulated with Rituximab biosimilar stock (9.3 mg/mL) and/or saline to give final concentrations of 7.9 mg/mL antibody and the indicated concentration of DES.

Stability evaluation of insulin-DESformulations using transmittance. Transmittance was used to assess insulin-DES formulation stability. Absorbance measurements taken at a wavelength of 540 nm with a BioTek Synergy neo2 Plate reader were converted to percent transmittance with Equation 1.

T(%)=10^{(2-A}^{₅₄₀}⁾

This equation relates absorbance to percent transmittance, derived from the Beer-Lambert Law.

The insulin-DES formations were first analyzed for initial transmittance to confirm that there was no insulin aggregation immediately after formulation preparation. Subsequently, formulations that passed initial screening were plated in a black-walled 96 well plate (replicates of three) and subjected to a stressed aging assay in which the plate was incubated at 37° C. with constant shaking. Absorbance readings were taken every 15 minutes for 50 hours to assess the change in transmittance over time. For cold chain stability, formulations were tested in a similar manner, without constant shaking, at 4° C. for 28 days.

Protein Stability Assessment Using Circular Dichroism and SDS-PAGE. Insulin and antibody formulations were incubated at 37° C. for one hour and subsequently dialyzed against 10 mM Sodium Phosphate pH 7.4. In preparation for circular dichroism (CD) and SDS-PAGE, the post-dialysis concentration was measured with UV Spectrophotometry (Thermo Scientific NanoDrop One) and adjusted to 200 μg/mL. CD was performed by loading quartz cuvettes (Starna Cells Spectrosil Quartz 1-Q-1), with 200 μL of the sample, into a CD spectrophotometer (Jasco J-815). Mean ellipticity was measured from 190 to 250 nm.

To assess antibody aggregation, SDS-PAGE was performed using vertical gel electrophoresis (BioRad Mini-PROTEAN Tetra Cell) loaded with precast polyacrylamide gels (BioRad TGX 4-15%) according to the manufacturer protocol. To form SDS complexes, samples were incubated in 1× Lammeli buffer for 10 minutes at 70° C. before loading in the gel. After 30 minutes of electrophoresis, the gel was removed and protein bands stained with Coumassie dye.

Transwell vascular permeability studies. To calculate the maximum concentration at which there was no cell death an MTS cell viability assay was performed. HUVECs were cultured according to supplier protocols, subcultured, and seeded on the in a 96 well-plate at a density of 10,000 cells/well. After allowing for cell adhesion overnight, the cells were treated with serial dilutions of DESs dissolved in fresh HUVEC media. The plate was incubated for 4 hours before the media was aspirated and replaced with fresh media containing 20% MTS Reagent. After 1 hour the absorbance was measured at 490 nm (BioTek Synergy neo2).

To assess vascular permeability, transport across transwell cell culture experiments were used. First, sufficient 24 well-plate transwell inserts were coated with 0.1% gelatin under sterile conditions and stored at 4° C. overnight. The excess gelatin was removed by inversion of transwell and washed with sterile PBS. HUVECs were cultured according to supplier protocols, subcultured, and seeded on the transwell inserts with 400 μL of 250,000 cells/mL media. The outer well was then filled with 600 μL of fresh media and allowed to grow for 48 hours before the experimental progression. After monolayers had formed, the media was removed from the top chamber and replaced with media containing 0.15% IL and 0.9 U/mL insulin, this was in preparation for the concentration and insulin to IL ratio to be used in the future in vivo studies. The plate was incubated inappropriate cell culture conditions and 300 μL samples were taken from the plate wells and replaced with fresh media every 10 min for 1 hour. The samples were then stored at 4° C. until they were diluted appropriately and quantified using ELISA.

Formulation Stability in the Presence of Extracellular Matrix Proteins. To evaluate the physical stability of the formulations, the hydrodynamic size of insulin and antibody was measured using dynamic light scattering (DLS, Malvern) in the presence of human collagen type I/III (Advanced BioMatrix, Carlsbad, CA). Insulin and Rituxan formulations with DESs, along with their respective clinical controls Humalog and Rituxan-saline, were added to a neutral-pH solution of collagen at pre-determined w/w ratios of collagen to insulin or antibody, and size was measured at various timepoints.

Physical stability of the formulations in the presence of collagen was further assessed using fluorescence polarization (FP). In principle, a molecule of interest, in this case insulin around 5.8 kDa, will rotate faster in an unbound state than a bound state, in this case bound to collagen around 300 kDa, due to its Brownian rotation and its smaller molecular radius. When insulin is fluorescently labeled and excited with polarized light the perpendicular and parallel polarized light emission can be measured. Plate readers designed for FP can measure perpendicular and parallel polarized light to generate a polarization, with units mP, value which indicates the binding state of the fluorescently labeled protein. Bound insulin that is rotating more slowly will emit in the parallel direction and have a higher polarization value as the complex has not had sufficient time to rotate. Unbound insulin, or binding inhibition, is indicated by more perpendicular binding and lower polarization values.³⁶ Formulations were prepared with Cy 5.5-labeled insulin. The formulations were mixed with a collagen solution at pre-determined w/w ratio, and fluorescence polarization was detected using Molecular Devices Flexstation 3 plate reader.

In vivo pharmacokinetic and bioavailability experiments experiments. All experiments were performed under institutionally approved protocols at Harvard University. The studies were performed in adult male Wistar non-fasting rats weighing between 350 and 550 g. The rats were anesthetized and blood was collected for t=0 timepoint into K2 EDTA collection tubes. After initial blood collection, the rats received subcutaneous injects in the neck scruff. For the insulin study, the rats were dosed with 1 U/kg of Humalog or SPADE-insulin formulation (both at a concentration of 3 U/mL). Blood was then collected at 5, 10, 15, 20, 30, 45, 60, 150, and 240 minutes after injection. Insulin concentrations were then quantified with the relevant ELISA kits.

For the antibody study, rats received right flank subcutaneous injections of 10 mg/kg. Blood samples on the first day were collected at 1, 3, 5, 8, and 11 hours after injection. The study continued and blood samples were collected once daily on days 1, 2, 3, 4, 7, 10, 14, 21, 28, 35, 42, and 49 after injection. Rituximab biosimilar concentrations were then quantified with ELISA.

In Vivo Toxicity. Healthy female BALB/c mice (aged 7-8 weeks) received a single subcutaneous injection of either DES solution or blank saline in the back scruff Mice were euthanized at 1 and 7 days post-injection, and the local skin tissue (from stratum corneum to muscle) was harvested, fixed in paraformaldehyde, and sectioned for H&E staining. Another cohort of mice received four daily subcutaneous injections of either DES solution or blank saline, euthanized 24 hour post the last treatment, and blood and major organs were harvested. Blood was collected into both K2 EDTA coated tubes, for whole blood analysis, and clot activator coated tubes, for serum analysis. The clot activator tubes were centrifuged at 2600rcf for 10 minutes to separate the serum from the clotted blood. Samples were kept on ice until hematology assays were run. After blood collection, mice were euthanized and vital organs were collected, washed with PBS, and fixed with 10% formalin. Whole blood and serum were analyzed for comprehensive complete blood count and blood chemistry (IDEXX BioAnalytics, North Grafton, MA), while organs were fixed and sectioned for H&E staining. All histological sections were imaged with AxioScan and interpreted by a professional histopathologist at the Rodent Histopathology Core at Harvard Medical School.

### Statistical Analysis

Unless otherwise specified, the data was plotted using GraphPad Prism 8 as mean±standard error of the mean (SEM). Statistical significance for experimental results that had more than two cohorts was assessed using analysis of variance (ANOVA) with Tukey's multiple comparisons, mean of each group was compared with the mean of every other group. Statistical significance for experimental resuhs that had exactly two cohorts was assessed using a two-tailed t-test. Significance marks were categorized with the following p-values: *p<0.05, **p<0.01, ***p<0.001, ****p<0.0001.

### REFERENCES

1. Mullard A. 2021 FDA approvals. Nat Rev Drug Discov. 2022.
2. Jin J F, Zhu L L, Chen M, et al. The optimal choice of medication administration route regarding intravenous, intramuscular, and subcutaneous injection. Patient Prefer Adherence. 2015; 9. doi:10.2147/PPA.S87271
3. de Cock E, Kritikou P, Sandoval M, et al. Time Savings with Rituximab Subcutaneous Injection versus Rituximab Intravenous Infusion: A time and motion study in eight countries. PLoS One. 2016; 11(6). doi:10.1371/journal.pone.0157957
4. Usach I, Martinez R, Festini T, Peris J E. Subcutaneous Injection of Drugs: Literature Review of Factors Influencing Pain Sensation at the Injection Site. Adv Ther. 2019; 36(11). doi:10.1007/s12325-019-01101-6
5. Overton P M, Shalet N, Somers F, Allen J A. Patient preferences for subcutaneous versus intravenous administration of treatment for chronic immune system disorders: A systematic review. Patient Prefer Adherence. 2021; 15. doi: 10.2147/PPA.S303279
6. Stoner K L, Harder H, Fallowfield L J, Jenkins V A. Intravenous versus Subcutaneous Drug Administration. Which Do Patients Prefer? A Systematic Review. Patient. 2015; 8(2). doi:10.1007/s40271-014-0075-y
7. Gandhi M D, Shapouri S, Ravelo A, Sudharshan L, Beeks A, Dawson K L. NHL Patients and Nurses in the US Prefer Subcutaneous Rituximab Injection Versus Intravenous Rituximab Infusion: A Real-World Study. Blood. 2020; 136(Supplement 1). doi:10.1182/blood-2020-136543
8. Frantz C, Stewart K M, Weaver V M. The extracellular matrix at a glance. J Cell Sci. 2010; 123(24). doi:10.1242/jcs.023820
9. Jones G B, Collins D S, Harrison M W, Thyagarajapuram N R, Wright J M. Subcutaneous drug delivery: An evolving enterprise. Sci Transl Med. 2017; 9(405). doi:10.1126/scitranslmed.aaf9166
10. Li C, Wan L, Luo J, Jiang M, Wang K. Advances in subcutaneous delivery systems of biomacromolecular agents for diabetes treatment. Int J Nanomedicine. 2021; 16. doi:10.2147/IJN.S283416
11. Brown S A, Kovatchev B P, Raghinaru D, et al. Six-Month Randomized, Multicenter Trial of Closed-Loop Control in Type 1 Diabetes. N Engl J Med. 2019; 381(18). doi:10.1056/nejmoa1907863
12. Rege N K, Liu M, Yang Y, et al. Evolution of insulin at the edge of foldability and its medical implications. Proc Natl Acad Sci USA. 2020; 117(47). doi: 10.1073/pnas.2010908117
13. Sanlioglu A D, Altunbas H A, Balci M K, Griffith T S, Sanlioglu S. Clinical utility of insulin and insulin analogs. Islets. 2013; 5(2). doi:10.4161/isl.24590
14. Datta-Mannan A, Witcher D R, Lu J, Wroblewski V J. Influence of improved FcRn binding on the subcutaneous bioavailability of monoclonal antibodies in cynomolgus monkeys. MAbs. 2012; 4(2). doi:10.4161/mabs.4.2.19364
15. Deng R, Meng Y G, Hoyte K, et al. Subcutaneous bioavailability of therapeutic antibodies as a function of FcRn binding affinity in mice. MAbs. 2012; 4(1). doi:10.4161/mabs.4.1.18543
16. Mann J L, Maikawa C L, Smith A A A, et al. An ultrafast insulin formulation enabled by high-throughput screening of engineered polymeric excipients. Sci Transl Med. 2020; 12(550). doi:10.1126/SCITRANSLMED.ABA6676
17. Webber M J, Appel E A, Vinciguerra B, et al. Supramolecular PEGylation of biopharmaceuticals. Proc Natl Acad Sci USA. 2016; 113(50). doi:10.1073/pnas.1616639113
18. Yang C, Lu D, Liu Z. How PEGylation enhances the stability and potency of insulin: A molecular dynamics simulation. Biochemistry. 2011; 50(13). doi:10.1021/bi101926u
19. Maikawa C L, Smith A A A, Zou L, et al. Stable Monomeric Insulin Formulations Enabled by Supramolecular PEGylation of Insulin Analogues. Adv Ther. 2020; 3(1). doi:10.1002/adtp.201900094
20. Liu Y, Lee J, Mansfield K M, et al. Trehalose Glycopolymer Enhances Both Solution Stability and Pharmacokinetics of a Therapeutic Protein. Bioconjug Chem. 2017; 28(3). doi:10.1021/acs.bioconjchem.6b00659
21. Awwad S, Angkawinitwong U. Overview of antibody drug delivery. Pharmaceutics. 2018; 10(3). doi: 10.3390/pharmaceutics10030083
22. Jiskoot W, Hawe A, Menzen T, Volkin D B, Crommelin DJA. Ongoing Challenges to Develop High Concentration Monoclonal Antibody-based Formulations for Subcutaneous Administration: Quo Vadis? J Pharm Sci. 2022; 111(4). doi:10.1016/j.xphs.2021.11.008
23. Kagan L, Mager D E. Mechanisms of subcutaneous absorption of rituximab in rats. Drug Metab Dispos. 2013; 41(1). doi:10.1124/dmd.112.048496
24. Turner M R, Balu-Iyer S V. Challenges and Opportunities for the Subcutaneous Delivery of Therapeutic Proteins. J Pharm Sci. 2018; 107(5). doi:10.1016/j.xphs.2018.01.007
25. Hamadani C M, Goetz M J, Mitragotri S, Tanner EEL. Protein-avoidant ionic liquid (pail)-coated nanoparticles to increase bloodstream circulation and drive biodistribution. Sci Adv. 2020; 6(48). doi:10.1126/sciadv.abd7563
26. Tanner EEL, Curreri A M, Balkaran J P R, et al. Design Principles of Ionic Liquids for Transdermal Drug Delivery. Adv Mater. 2019; 31(27). doi: 10.1002/adma.201901103
27. Banerjee A, Ibsen K, Brown T, Chen R, Agatemor C, Mitragotri S. Ionic liquids for oral insulin delivery. Proc Natl Acad Sci USA. 2018; 115(28). doi:10.1073/pnas.1722338115
28. Angsantikul P, Peng K, Curreri A M, et al. Ionic Liquids and Deep Eutectic Solvents for Enhanced Delivery of Antibodies in the Gastrointestinal Tract. Adv Funct Mater. 2021; 31(44). doi:10.1002/adfm.202002912
29. Vaidya A, Mitragotri S. Ionic liquid-mediated delivery of insulin to buccal mucosa. J Control Release. 2020; 327. doi:10.1016/j.jconrel.2020.07.037
30. Tanner E E L, Ibsen K N, Mitragotri S. Transdermal insulin delivery using choline-based ionic liquids (CAGE). J Control Release. 2018; 286. doi:10.1016/j.jconrel.2018.07.029
31. Banerjee A, Ibsen K, Iwao Y, Zakrewsky M, Mitragotri S. Transdermal Protein Delivery Using Choline and Geranate (CAGE) Deep Eutectic Solvent. Adv Healthc Mater. 2017; 6(15). doi:10.1002/adhm.201601411
32. Kim J, Gao Y, Zhao Z, et al. A deep eutectic-based, self-emulsifying subcutaneous depot system for apomorphine therapy in Parkinson's disease. Proc Natl Acad Sci. 2022; 119(9). doi:10.1073/pnas.2110450119
33. Yu Y B, Briggs K T, Taraban M B, Brinson R G, Marino J P. Grand Challenges in Pharmaceutical Research Series: Ridding the Cold Chain for Biologics. Pharm Res. 2021; 38(1). doi:10.1007/s11095-021-03008-w
34. Rossi A M, Taylor C W. Analysis of protein-ligand interactions by fluorescence polarization. Nat Protoc. 2011; 6(3). doi:10.1038/nprot.2011.305
35. Lea W A, Simeonov A. Fluorescence polarization assays in small molecule screening. Expert Opin Drug Discov. 2011; 6(1). doi:10.1517/17460441.2011.537322
36. Moerke N J. Fluorescence Polarization (FP) Assays for Monitoring Peptide-Protein or Nucleic Acid-Protein Binding. Curr Protoc Chem Biol. 2009; 1(1). doi:10.1002/9780470559277.ch090102
37. Fischer A H, Jacobson K A, Rose J, Zeller R. Hematoxylin and eosin staining of tissue and cell sections. Cold Spring Harb Protoc. 2008; 3(5). doi:10.1101/pdb.prot4986
38. BALB/C Mouse Hematology. Charles River.
39. Aulbach A D, Amuzie C J. A Comprehensive Guide to Toxicology in Nonclinical Drug Development. Second.; 2017.
40. Walls R M. Emergency Medicine: Concepts and Clinical Practice. Ronsen; 2018.
41. Maikawa C L, Chen P C, Vuong E T, et al. Ultra-Fast Insulin-Pramlintide Co-Formulation for Improved Glucose Management in Diabetic Rats. Adv Sci. 2021; 8(21). doi:10.1002/advs.202101575
42. Vaughn D, Muchmore D. Use of recombinant human hyaluronidase to accelerate rapid insulin analogue absorption: Experience with subcutaneous injection and continuous infusion. Endocr Pract. 2011; 17(6). doi:10.4158/EP11297.RA
43. Tannous E, Kanaya E, Kanaya S. Role of RNase H1 in DNA repair: Removal of single ribonucleotide misincorporated into DNA in collaboration with RNase H2. Sci Rep. 2015; 5. doi:10.1038/srep09969

TABLE 4
Table of DES anion properties

| Acid Name | pKa | log(P) | MW (g/mol) | Carbon Chain |
|---|---|---|---|---|
| Glycolic | 3.83 | −1.11 | 76.06 | 2 |
| Lactic | 3.86 | −0.72 | 90.08 | 3 |
| Propionic | 4.33 | 0.33 | 74.08 | 3 |
| Hexenoic | 4.74 | 1.81 | 114.14 | 6 |
| Geranic | 5.26 | 2.82 | 168.23 | 8 |

TABLE 5
Table of deep eutectic solvent abbreviations

| Abbreviation | Full Name |
|---|---|
| CG | cholinium glycolate DES |
| aCG | acetylcholinium glycolate DES |
| CL | cholinium lactate DES |
| aCL | acetylcholinium lactate DES |
| CP | cholinium propionate DES |
| aCP | acetylcholinium priopionate DES |
| CH | cholinium hexenoate DES |
| aCH | acetylcholinium hexenoate DES |
| CAGE | cholinium geranate DES |
| aCAGE | acetylcholinium geranate DES |

TABLE 6
Table of statistical significance values (p-value) of the t-tests performed
on the serum concentrations and area under the curve (AUC) values
for all timepoints in the SPADE-mAb bioavailability study.

| | Time | Serum Concentration | AUC |
|---|---|---|---|
| Hours | 1 | <0.0001**** | <0.0001**** |
| | 2 | 0.0029*** | 0.0003*** |
| | 5 | 0.0074** | 0.0025** |
| | 8 | 0.0261* | 0.0062** |
| | 11 | 0.0512 | 0.0109* |
| Days | 1 | 0.0263* | 0.0205* |
| | 2 | 0.022* | 0.02* |
| | 3 | 0.0164* | 0.0152* |
| | 4 | 0.0498* | 0.0162* |
| | 7 | 0.0826* | 0.0288* |
| | 10 | 0.0193* | 0.0279* |
| | 14 | 0.0288* | 0.0215* |
| | 21 | N/A | 0.0159* |
| | 28 | N/A | 0.027* |
| | 35 | N/A | 0.0335* |
| | 42 | N/A | 0.0368* |
| | 49 | N/A | 0.0397* |

Significance marks were categorized with the following p-values:
*p < 0.05,
**p < 0.01,
***p < 0.001,
****p < 0.0001.

### REFERENCES

1. Hughes B. 2007 FDA drug approvals: a year of flux. Nat Rev Drug Discov. 2008; 7(2). doi:10.1038/nrd2514
2. Hughes B. 2008 FDA drug approvals. Nat Rev Drug Discov. 2009; 8(2). doi:10.1038/nrd2813
3. Hughes B. 2009 FDA drug approvals. Nat Rev Drug Discov. 2010; 9(2). doi:10.1038/nrd3101
4. Mullard A. 2010 FDA drug approvals. Nat Rev Drug Discov. 2011; 10(2). doi:10.1038/nrd3370
5. Mullard A. 2011 FDA drug approvals. Nat Rev Drug Discov. 2012; 11(2). doi: 10.1038/nrd3657
6. Mullard A. 2012 FDA drug approvals. Nat Rev Drug Discov. 2013; 12(2). doi:10.1038/nrd3946
7. Mullard A. 2013 FDA drug approvals. Nat Rev Drug Discov. 2014; 13(2). doi:10.1038/nrd4239
8. Mullard A. 2014 FDA drug approvals. Nat Rev Drug Discov. 2015; 14(2). doi:10.1038/nrd4545
9. Mullard A. 2015 FDA drug approvals. Nat Rev Drug Discov. 2016; 15(2). doi:10.1038/nrd.2016.15
10. Mullard A. 2016 FDA drug approvals. Nat Rev Drug Discov. 2017; 16(2). doi:10.1038/nrd.2017.14
11. Mullard A. 2017 FDA drug approvals. Nat Rev Drug Discov. 2018; 17(2). doi:10.1038/nrd.2018.4
12. Mullard A. 2018 FDA drug approvals. Nat Rev Drug Discov. 2019; 18(2). doi:10.1038/d41573-019-00014-x
13. Mullard A. 2019 FDA drug approvals. Nat Rev Drug Discov. 2020; 19(2). doi:10.1038/d41573-020-00001-7
14. Mullard A. 2020 FDA drug approvals. Nat Rev Drug Discov. 2021; 20(2). doi:10.1038/d41573-021-00002-0
15. Mullard A. 2021 FDA approvals. Nat Rev Drug Discov. 2022.

What is claimed herein is:

1. A composition comprising at least one ionic liquid comprising:
an anion which is at least one of:
a) a carboxylic acid which is not a fatty acid; and
b) a hydrophobic anion comprising carboxylic acid having a pKa of at least 4.0 and a Log P of at least 1.0;
a cation which is a quaternary ammonium comprising an ester group.

2. The composition of claim 1, wherein the cation is acetylcholine.

3. The composition of any one of claims 1-2, wherein the anion is a carboxylic acid which is not a fatty acid.

4. The composition of claim 3, wherein the anion has a Log P of less than 1.0.

5. The composition of any one of claim 3-4, wherein the anion comprises an aliphatic chain of no more than 3 carbons.

6. The composition of any one of claims 3-5, wherein the anion comprises only one carboxylic acid group (e.g., R—COOH group).

7. The composition of any one of claims 3-6, wherein the anion is selected from the group consisting of:
lactic acid; glycolic acid; malonic acid; maleic acid; glutaric acid; citric acid;
gluconic acid; propanoic acid; and adipic acid.

8. The composition of any one of claims 3-7, wherein the anion is maleic acid.

9. The composition of any one of claims 3-8, wherein the anion is propanoic acid.

10. The composition of any one of claims 1-2, wherein the anion is a hydrophobic anion comprising carboxylic acid having a pKa of at least 4.0 and a Log P of at least 1.0 and has a pKa of at least 4.5.

11. The composition of claim 10, wherein the anion has a pKa of at least 5.0.

12. The composition of any one of claims 10-11, wherein the anion comprises a carbon chain of at least 8 carbons.

13. The composition of any one of claims 10-12, wherein the anion comprises a carbon chain with an 8 carbon backbone.

14. The composition of any one of claims 10-13, wherein the anion is geranic acid, octenoic acid, octanoic acid, citronellic acid, decenoic acid, (9Z)-octadec-9-enoic acid, decanoic acid, (9Z,12Z)-octadeca-9,12-dienoic acid, (R)-5-(1,2-dithiolan-3-yl)pentanoic acid, or hexenoic acid.

15. The composition of any one of claims 10-14, wherein the anion is hexenoic acid.

16. The composition of any one of the preceding claims, wherein the ionic liquid comprises a ratio of cation to anion of from about 2:1 to about 1:1.

17. The composition of any one of the preceding claims, wherein the ionic liquid comprises a ratio of cation to anion of about 2:1.

18. The composition of any one of the preceding claims, wherein the ionic liquid has a cation:anion ratio of less than 1:1.

19. The composition of any one of the preceding claims, wherein the ionic liquid has a cation:anion ratio with an excess of cation.

20. The composition of any one of the preceding claims, comprising a first ionic liquid and at least a second ionic liquid.

21. The composition of claim 20, wherein the first ionic liquid and the second ionic liquid each comprise a different anion.

22. The composition of any one of the preceding claims, further comprising at least one active compound in combination with the at least one ionic liquid.

23. The composition of claim 22, wherein the active compound comprises a polypeptide.

24. The composition of claim 23, wherein the polypeptide is an antibody or antibody reagent.

25. The composition of any one of claims 22-24, wherein the active compound has a molecular weight of greater than 450.

26. The composition of any one of claims 22-25, wherein the active compound has a molecular weight of greater than 500.

27. The composition of any one of claims 22-26, wherein the active compound comprises insulin, acarbose, ruxolitinib, or a GLP-1 polypeptide or mimetic or analog thereof.

28. The composition of any one of claims 22-27, wherein the anion is a hydrophobic anion comprising carboxylic acid having a pKa of at least 4.0 and a Log P of at least 1.0, and the active compound comprises an antibody or antibody reagent.

29. The composition of any one of claims 22-28, wherein the anion is hexenoic acid, and the active compound comprises an antibody or antibody reagent.

30. The composition of any one of claims 22-29, wherein the anion is a hydrophobic anion comprising carboxylic acid having a pKa of at least 4.0 and a Log P of at least 1.0, the ionic liquid is present at a concentration of less than 10% w/v, and the active compound comprises an antibody or antibody reagent.

31. The composition of any one of claims 22-30, wherein the anion is hexenoic acid, the ionic liquid is present at a concentration of less than 10% w/v, and the active compound comprises an antibody or antibody reagent.

32. The composition of claim 22, wherein the active compound comprises a nucleic acid.

33. The composition of claim 32, wherein the nucleic acid is an inhibitory nucleic acid.

34. The composition of claim 32 or 33, wherein the nucleic acid is a siRNA, pDNA, or mRNA.

35. The composition of any one of claims 32-34, wherein the anion is a hydrophobic anion comprising carboxylic acid having a pKa of at least 4.0 and a Log P of at least 1.0 and the active compound comprises a nucleic acid.

36. The composition of any one of the preceding claims, wherein the ionic liquid is at a concentration of at least 0.1% w/v.

37. The composition of any one of the preceding claims, wherein the ionic liquid is at a concentration of from about 10 to about 70% w/v.

38. The composition of any one of the preceding claims, wherein the ionic liquid is at a concentration of from about 30 to about 50% w/v.

39. The composition of any one of the preceding claims, wherein the ionic liquid is at a concentration of from about 30 to about 40% w/v.

40. The composition of any one of the preceding claims, wherein the ionic liquid is at a concentration of less than 10% w/v.

41. The composition of any one of the preceding claims, wherein the composition is formulated for administration transdermally, to a mucus membrane, orally, subcutaneously, intradermally, parenterally, intratumorally, or intravenously.

42. The composition of claim 41, wherein the composition is formulated for subcutaneous administration.

43. The composition of claim 41, wherein the composition is formulated for transdermal administration.

44. The composition of claim 41, wherein the mucus membrane is nasal, oral, or vaginal.

45. The composition of any one of the preceding claims, wherein the active compound is provided at a dosage of 1-40 mg/kg.

46. The composition of any one of the preceding claims, further comprising at least one non-ionic surfactant.

47. The composition of any one of the preceding claims, further comprising a pharmaceutically acceptable carrier.

48. The composition of any one of the preceding claims, wherein the composition is provided in a degradable capsule.

49. The composition of any one of the preceding claims, wherein the composition is an admixture.

50. The composition of any one of the preceding claims, wherein the composition is provided in one or more nanoparticles.

51. The composition of any one of the preceding claims, comprising one or more nanoparticles comprising the active compound, the nanoparticles in solution or suspension in a composition comprising the ionic liquid.

52. A method of administering at least one active compound to a subject, the method comprising administering a composition of any one of claims 1-51.

53. The method of claim 52, wherein the composition is administered once.

54. The method of any one of claims 52-53, wherein the composition is administered in multiple doses.

55. The method of any one of claims 52-54, wherein the administering is transdermally, to a mucus membrane, orally, subcutaneously, intradermally, parenterally, intratumorally, or intravenously.

56. The method of any one of claims 52-55, wherein the administering is subcutaneous.

57. A composition of any one of claims 1-51 for use in a method of administering at least one active compound to a subject.

58. The composition of claim 57, wherein the composition is administered once.

59. The composition of any one of claims 57-58, wherein the composition is administered in multiple doses.

60. The composition of any one of claims 57-59, wherein the administering is transdermally, to a mucus membrane, orally, subcutaneously, intradermally, parenterally, intratumorally, or intravenously.

61. The composition of any one of claims 57-60, wherein the administering is subcutaneous.
